SEQUENCE LISTING (15 sequences)

SEQ ID NO: 1 - DNA, 977 bp, Bradyrhizobium sp.
  misc_feature 1..977: note = BRADYRHIZOBIUM_SP_CRTW
  source 1..977: mol_type = genomic DNA
atgcacgcagccactgccaaagccacagagtttggtgcctcccgtagagacgatgctagacaaagaagagtcggtttgactttagctgccgttatcatcgctgcttggttggtcttgcacgtcggtttgatgttcttctggccattgactttgcactctttgttaccagctttaccattggtcgtcttgcaaacttggttgtacgttggtttgttcattattgctcacgactgtatgcacggttctttggttccattcaaacctcaagtcaacagaagaatcggtcaattgtgcttgtttttgtacgctggtttctctttcgacgctttaaacgttgaacaccacaaacatcatcgtcatccaggtactgctgaagatcctgattttgacgaagtccctccacatggtttttggcattggttcgcttcctttttcttgcactacttcggttggaaacaagttgccattatcgctgccgtctccttagtttaccaattagtttttgctgtcccattgcaaaacatcttattgttctgggctttacctggtttattgtccgccttgcaattgttcactttcggtacttatttaccacataagccagctacccaaccttttgctgacagacataacgccagaacctctgaattcccagcctggttgtctttattgacctgtttccacttcggtttccaccacgaacaccatttgcatccagacgctccatggtggcgtttaccagaaatcaagagacgtgccttggaaagaagagactaagagtatgcttctctttttttttgtaggccagtgataggaaagaacaatagaatataaatacgtcagaatataatagatatgtttttatatttagacctcgtacataggaataattgacgttttttttggccaacatttgaaatttttttttgttacctcgcgctgagcccaaacgggctccactacccgccgcggtcgcc

SEQ ID NO: 2 - protein, 258 aa, Bradyrhizobium sp.
  REGION 1..258: note = misc_feature - Bradyrhizobium Beta-carotene ketolase
MHAATAKATEFGASRRDDARQRRVGLTLAAVIIAAWLVLHVGLMFFWPLTLHSLLPALPLVVLQTWLYVGLFIIAHDCMHGSLVPFKPQVNRRIGQLCLFLYAGFSFDALNVEHHKHHRHPGTAEDPDFDEVPPHGFWHWFASFFLHYFGWKQVAIIAAVSLVYQLVFAVPLQNILLFWALPGLLSALQLFTFGTYLPHKPATQPFADRHNARTSEFPAWLSLLTCFHFGFHHEHHLHPDAPWWRLPEIKRRALERRD

SEQ ID NO: 3 - DNA, 979 bp, Paracococus sp.
  misc_feature 1..979: note = Paracococus sp. Beta-carotene ketolase
  source 1..979: mol_type = genomic DNA
atgtcagcacacgcacttcctaaagcagatttgactgctacttctttgatcgtttctggtggtattattgctgcctggttagccttgcacgttcacgctttgtggttcttagacgctgctgctcatccaattttagctattgctaattttttgggtttaacttggttgtccgttggtttgttcattattgcccacgacgctatgcacggttctgtcgtccctggtagaccaagagccaacgctgccatgggtcaattagttttgtggttgtacgccggtttctcctggcgtaagatgatcgttaagcatatggctcaccatcgtcacgctggtaccgacgacgatcctgacttcgaccacggtggtccagttagatggtacgccagatttatcggtacttacttcggttggagagaaggtttgttgttgccagttatcgtcaccgtttacgctttaatcttgggtgatagatggatgtacgttgtcttctggccattgccatctattttggcctctattcaattattcgtctttggtacctggttgccacacagaccaggtcacgacgctttcccagacagacacaacgcccgttcttctagaatctctgatccagtctctttattgacctgtttccatttcggtggttaccatcatgaacatcacttacacccaactgtcccatggtggagattgccatccactagaactaaaggtgacactgcttaaattgaattgaattgaaatcgatagatcaatttttttcttttctctttccccatcctttacgctaaaataatagtttattttattttttgaatattttttatttatatacgtatatatagactattatttatcttttaatgattattaagatttttattaaaaaaaaattcgctcctcttttaatgcctttatgcagtttttttttcccattcgatatttctatgttcgggttcagcgtattttaagttta

SEQ ID NO: 4 - protein, 242 aa, Paracococus sp.
  REGION 1..242: note = misc_feature - Paracoccus Beta-carotene ketolase
MSAHALPKADLTATSLIVSGGIIAAWLALHVHALWFLDAAAHPILAIANFLGLTWLSVGLFIIAHDAMHGSVVPGRPRANAAMGQLVLWLYAGFSWRKMIVKHMAHHRHAGTDDDPDFDHGGPVRWYARFIGTYFGWREGLLLPVIVTVYALILGDRWMYVVFWPLPSILASIQLFVFGTWLPHRPGHDAFPDRHNARSSRISDPVSLLTCFHFGGYHHEHHLHPTVPWWRLPSTRTKGDTA

SEQ ID NO: 5 - DNA, 1008 bp, Saccharomyces cerevisiae
  misc_feature 1..1008: note = Geranylgeranyl diphosphate synthase
  source 1..1008: mol_type = genomic DNA
atggaggccaagatagatgagctgatcaataatgatcctgtttggtccagccaaaatgaaagcttgatttcaaaaccttataatcacatccttttgaaacctggcaagaactttagactaaatttaatagttcaaattaacagagttatgaatttgcccaaagaccagctggccatagtttcgcaaattgttgagctcttgcataattccagccttttaatcgacgatatagaagataatgctcccttgagaaggggacagaccacttctcacttaatcttcggtgtaccctccactataaacaccgcaaattatatgtatttcagagccatgcaacttgtatcgcagctaaccacaaaagagcctttgtatcataatttgattacgattttcaacgaagaattgatcaatctacataggggacaaggcttggatatatactggagagactttctgcctgaaatcatacctactcaggagatgtatttgaatatggttatgaataaaacaggcggccttttcagattaacgttgagactcatggaagcgctgtctccttcctcacaccacggccattcgttggttcctttcataaatcttctgggtattatttatcagattagagatgattacttgaatttgaaagatttccaaatgtccagcgaaaaaggctttgctgaggacattacagaggggaagttatcttttcccatcgtccacgcccttaacttcactaaaacgaaaggtcaaactgagcaacacaatgaaattctaagaattctcctgttgaggacaagtgataaagatataaaactaaagctgattcaaatactggaattcgacaccaattcattggcctacaccaaaaattttattaatcaattagtgaatatgataaaaaatgataatgaaaataagtatttacctgatttggcttcgcattccgacaccgccaccaatttacatgacgaattgttatatataatagaccacttatccgaattgtga

SEQ ID NO: 6 - protein, 335 aa, Saccharomyces cerevisiae [sequence omitted]

SEQ ID NO: 7 - DNA, 2516 bp, Xanthophyllomyces dendrorhous
  misc_feature 1..2516: note = yeast codon-optimized XdCrtYB, including NC1 terminator
  source 1..2516: mol_type = genomic DNA
atgaccgccttggcctactatcaaattcatttgatttacactttgccaatcttgggtttgttgggtttattgacttctccaattttaactaagttcgacatctacaagatctctattttggttttcattgccttttctgctactactccatgggattcttggattattcgtaacggtgcttggacttacccatctgctgaatccggtcaaggtgtttttggtacttttttggacgttccatacgaagaatacgcctttttcgttatccaaactgttatcaccggtttggtctacgtcttggccactagacacttgttaccatctttggctttaccaaaaactagatcctctgctttgtctttagccttgaaggccttgattccattgccaattatctatttgtttaccgctcatccatctccatctccagatccattggtcaccgaccactacttctacatgagagctttgtctttattgatcactcctccaactatgttgttggccgctttgtccggtgaatatgctttcgattggaagtctggtagagccaaatctaccatcgctgccattatgattcctactgtctacttgatctgggtcgactacgttgccgttggtcaagattcctggtctattaatgatgaaaagatcgttggttggcgtttgggtggtgttttgccaattgaagaagctatgttcttcttgttgaccaacttgatgattgtcttaggtttatccgcttgtgaccatactcaagctttgtacttgttgcacggtcgtactatctacggtaacaagaaaatgccatcctccttcccattgattaccccaccagtcttgtctttgttcttctcttctagaccatactcttcccaaccaaagagagacttagagttagctgttaagttgttagaagaaaagtccagatctttcttcgtcgcttccgctggttttccatccgaggttagagaacgtttggttggtttgtacgccttctgtagagtcaccgatgatttgattgactctccagaggtttcttccaatccacacgccaccatcgacatggtttccgatttcttgaccttgttgttcggtccaccattacacccatctcaaccagacaagattttgtcttctcctttgttgccaccatcccacccatctagaccaaccggtatgtatccattgccaccaccaccatctttgtctccagctgaattggttcaattcttaactgaaagagtccctgttcaatatcactttgcttttagattgttggccaagttacaaggtttgattccaagatacccattggacgaattgttgagaggttacactaccgatttaatcttcccattgtctactgaagctgttcaagccagaaagaccccaattgaaactaccgctgatttattggactacggtttatgtgtcgccggttctgttgctgaattattggtctacgtttcttgggcttccgctccttcccaagttccagctactattgaagaaagagaggctgtcttagttgcctctagagaaatgggtaccgctttgcaattggttaacattgctagagatatcaagggtgatgccactgaaggtagattctacttgccattgtccttttttggtttgagagacgaatctaagttagctattccaactgattggaccgaaccaagaccacaagattttgacaagttgttgtctttgtccccatcttctactttaccatcttctaacgcttctgaatctttcagatttgaatggaagacttactccttgccattggttgcttatgctgaagatttggctaaacactcctacaagggtattgatagattgccaaccgaggttcaagccggtatgagagctgcctgtgcttcctacttgttgatcggtagagaaattaaggttgtttggaagggtgacgtcggtgaaagaagaactgttgctggttggagacgtgtccgtaaagttttgtctgttgtcatgtctggttgggaaggtcaatagttttagcctcaccgatatacttatataatattcattgaaatagaaagacatttacaaagtagacggggatatttagccccactcatataaattatgcattataacttttataaaaaaaaagaaatcacacttaaataaatgtcttaattgcacattccatgttcagatatacactatcttaccttttgaataggaatgatggaaaaaaaattatgcttccaatcggatttgaaccgatgatctccacattactagtgtggcgccttaccaacttggccatagaagcccgttattacggtctgtgatccagcaaatgtcagatataacgaattgaagttgcttttcccatcaatgtttgtcaagttgaaacgctgtagcacacactaagcattaacgactatattacaagtaaagcatcaaatgattacttaaaagagtaatcgcaaactatattgaagttggcaaaggcacatgatccaaatggtaacgataac

SEQ ID NO: 8 - protein, 673 aa, Xanthophyllomyces dendrorhous
  REGION 1..673: note = misc_feature - Xanthophyllomyces dendrorhous CrtYB
MTALAYYQIHLIYTLPILGLLGLLTSPILTKFDIYKISILVFIAFSATTPWDSWIIRNGAWTYPSAESGQGVFGTFLDVPYEEYAFFVIQTVITGLVYVLATRHLLPSLALPKTRSSALSLALKALIPLPIIYLFTAHPSPSPDPLVTDHYFYMRALSLLITPPTMLLAALSGEYAFDWKSGRAKSTIAAIMIPTVYLIWVDYVAVGQDSWSINDEKIVGWRLGGVLPIEEAMFFLLTNLMIVLGLSACDHTQALYLLHGRTIYGNKKMPSSFPLITPPVLSLFFSSRPYSSQPKRDLELAVKLLEEKSRSFFVASAGFPSEVRERLVGLYAFCRVTDDLIDSPEVSSNPHATIDMVSDFLTLLFGPPLHPSQPDKILSSPLLPPSHPSRPTGMYPLPPPPSLSPAELVQFLTERVPVQYHFAFRLLAKLQGLIPRYPLDELLRGYTTDLIFPLSTEAVQARKTPIETTADLLDYGLCVAGSVAELLVYVSWASAPSQVPATIEEREAVLVASREMGTALQLVNIARDIKGDATEGRFYLPLSFFGLRDESKLAIPTDWTEPRPQDFDKLLSLSPSSTLPSSNASESFRFEWKTYSLPLVAYAEDLAKHSYKGIDRLPTEVQAGMRAACASYLLIGREIKVVWKGDVGERRTVAGWRRVRKVLSVVMSGWEGQ

SEQ ID NO: 9 - DNA, 2252 bp, Xanthophyllomyces dendrorhous
  misc_feature 1..2252: note = yeast codon-optimized XdCrtI, including two QCR10 terminators
  source 1..2252: mol_type = genomic DNA
atgggtaaagaacaagaccaagataagccaactgctatcattgttggttgtggtattggtggtatcgctaccgccgctagattggctaaagaaggtttccaagttactgtcttcgaaaagaacgattattctggtggtagatgctctttgattgaaagagatggttatcgtttcgaccaaggtccatctttgttgttgttgccagacttgttcaagcaaaccttcgaagatttgggtgaaaagatggaagattgggtcgatttgatcaagtgtgaaccaaactacgtttgtcattttcatgatgaagaaaccttcactttctctaccgacatggctttattgaagagagaagtcgaaagattcgaaggtaaggatggtttcgatagatttttatctttcattcaagaagctcatagacactacgagttggccgttgttcatgtcttacaaaagaacttcccaggtttcgctgctttcttgagattgcaatttatcggtcaaattttggctttacatccatttgaatctatctggactagagtttgtagatacttcaagaccgatagattgagaagagttttttccttcgctgttatgtacatgggtcaatctccatactctgcccctggtacctactccttgttacaatatactgaattgactgaaggtatctggtatccacgtggtggtttttggcaagtcccaaacaccttattacaaatcgtcaaaagaaacaacccatctgctaagttcaacttcaacgctccagtttctcaagtcttgttgtccccagctaaggatagagctactggtgttagattggaatccggtgaggaacatcatgccgacgttgtcatcgtcaacgctgacttggtctacgcttctgaacacttaattccagatgacgccagaaataaaatcggtcaattgggtgaagtcaagagatcttggtgggccgatttggtcggtggtaagaagttaaaaggttcttgttcctctttatccttttactggtctatggatagaattgtcgatggtttgggtggtcataacattttcttggctgaagacttcaagggttcttttgacaccattttcgaggaattaggtttgccagctgacccatctttctacgttaacgtcccatccagaattgacccatctgctgctcctgaaggtaaagacgctatcgttattttagtcccatgtggtcacattgacgcctctaacccacaagactacaataagttagtcgctagagccagaaaattcgttattcaaaccttgtccgctaaattgggtttgccagacttcgaaaagatgattgttgccgaaaaggttcacgacgctccatcctgggaaaaggaattcaatttgaaagatggttccatcttgggtttggcccacaacttcatgcaagtcttgggtttcagaccatctaccagacacccaaaatacgacaagttgttttttgtcggtgcctctactcacccaggtaccggtgttcctatcgttttggctggtgccaagttaactgctaatcaagttttggaatctttcgatcgttccccagccccagatccaaacatgtctttgtctgttccatatggtaagccattgaagtctaacggtaccggtattgactcccaagtccaattgaagtttatggacttggaacgttgggtttacttattggtcttattgattggtgctgtcattgctagatccgtcggtgtcttggctttctgaagccaaaacattcaaccggctgagtgattttatttatttgttttacgttcttttctatcattttggaagaggaattaatgcaaaacacatttgtatttttttttatttttgcatattctcgattatctatacatatttttaaactggatcataaaaggtataaaaagtaattgatccatacaacatttttaaatatttacccctctattttcagaatatttactaaaatatcagttatcaaagagtagggaaaagttaactacaagtttgtttttcctctttttatgtatcttttgagctttgtaattaggcagtacatctctatgttttcagggaaacctatttcattcgccattttgatgatttaggcatacgccaatgctaacgccgtttcttgtaatttctattttttcaaatggagtactttaccttcctcccgcagctgaaactttcaacccgccggaagaaaagaccaaataatattataattaatatttaaaacc

SEQ ID NO: 10 - protein, 582 aa, Xanthophyllomyces dendrorhous
  REGION 1..582: note = misc_feature - Xanthophyllomyces dendrorhous CrtI
MGKEQDQDKPTAIIVGCGIGGIATAARLAKEGFQVTVFEKNDYSGGRCSLIERDGYRFDQGPSLLLLPDLFKQTFEDLGEKMEDWVDLIKCEPNYVCHFHDEETFTFSTDMALLKREVERFEGKDGFDRFLSFIQEAHRHYELAVVHVLQKNFPGFAAFLRLQFIGQILALHPFESIWTRVCRYFKTDRLRRVFSFAVMYMGQSPYSAPGTYSLLQYTELTEGIWYPRGGFWQVPNTLLQIVKRNNPSAKFNFNAPVSQVLLSPAKDRATGVRLESGEEHHADVVIVNADLVYASEHLIPDDARNKIGQLGEVKRSWWADLVGGKKLKGSCSSLSFYWSMDRIVDGLGGHNIFLAEDFKGSFDTIFEELGLPADPSFYVNVPSRIDPSAAPEGKDAIVILVPCGHIDASNPQDYNKLVARARKFVIQTLSAKLGLPDFEKMIVAEKVHDAPSWEKEFNLKDGSILGLAHNFMQVLGFRPSTRHPKYDKLFFVGASTHPGTGVPIVLAGAKLTANQVLESFDRSPAPDPNMSLSVPYGKPLKSNGTGIDSQVQLKFMDLERWVYLLVLLIGAVIARSVGVLAF

SEQ ID NO: 11 - DNA, 1152 bp, Haematococcus lacustris
  misc_feature 1..1152: note = Beta-carotene ketolase
  source 1..1152: mol_type = genomic DNA
atgcacgtagccagtgctttaatggtagaacaaaagggttctgaagccgctgcttcttctccagatgttttgagagcttgggccactcaataccacatgccatccgaatcctctgacgctgccagaccagctttgaagcacgcttacaagccaccagcctctgacgctaagggtattactatggccttaactattattggtacctggactgctgttttcttgcacgccatctttcaaattagattgccaacttccatggaccaattgcactggttgccagtttccgaagccaccgctcaattgttaggtggttcttcctccttgttacatattgccgctgtcttcattgttttggaatttttatacaccggtttgttcattaccacccatgacgctatgcatggtactatcgccttgagacacagacaattaaatgacttattgggtaacatctgtatctctttgtacgcttggttcgactactctatgttgcatagaaagcactgggaacaccacaaccacactggtgaagttggtaaagacccagactttcataagggtaaccctggtttggttccatggtttgcttccttcatgtcttcttacatgtccttgtggcaattcgcccgtttggcttggtgggccgttgttatgcaaatgttaggtgccccaatggctaacttgttggttttcatggctgctgctccaatcttgtctgctttcagattgttctactttggtacctacttgcctcacaagccagaaccaggtccagctgctggttcccaagttatggcttggttcagagctaagacttccgaggcttctgacgttatgtcctttttgacctgttaccattttgatttacactgggaacaccacagatggccattcgccccatggtggcaattacctcactgtagaagattgtccggtagaggtttggttccagctttggcctaagagtatgcttctctttttttttgtaggccagtgataggaaagaacaatagaatataaatacgtcagaatataatagatatgtttttatatttagacctcgtacataggaataattgacgttttttttggccaacatttgaaatttttttttgttacctcgcgctgagcccaaacgggctccactacccg

SEQ ID NO: 12 - DNA, 989 bp, Fulvimarina pelagi
  misc_feature 1..989: note = F_pelagi_ketolase_CrtW
  source 1..989: mol_type = genomic DNA
atgactttatctcctacttctcgtttaatcccagcctccgccttgccacgttccactccagccgactctccaaagatcagaccataccaaactaccatcggtttgactttgtgtgctgttttgttggcttcttggtttgctattcacgtttccgctatcttcttcttagatatcaacttctctactttgccattagccccattgattaccgtctttcaatgttggttgactgtcggtttatttattttggctcatgacgccatgcatggttctttggctccaggtagaactcgtttgaacgctgttattggtggtttcattttgtttgtttatgccggtttcgcttggaaaaagattagagatgcccacttcgctcaccacgatgctcctggtacccctgctgatccagacttctacgctgatgacccagaaaatttctggccatggttcggtaccttcttttctagatacttcggttggagatctgtcgccttcgtttctactgttgtcaccttctacttagttattttggatgcctctgtcactaacgtcgtcttgttctatggtttgccttccttgttgtcttccttgcaattgttttacttcggtacttacagaccacacagacacgaagaatctggtactttcgccgatgcccacaacactcgttcttctgaattcggttatgttgcttccttgttctcctgcttccacttcggttaccaccacgaacatcacttggccccatggactccttggtgggctttacctcatactagacaatcttaagagtatgcttctctttttttttgtaggccagtgataggaaagaacaatagaatataaatacgtcagaatataatagatatgtttttatatttagacctcgtacataggaataattgacgttttttttggccaacatttgaaatttttttttgttacctcgcgctgagcccaaacgggctccactacccgccgcggtcgcc

SEQ ID NO: 13 - DNA, 935 bp, unidentified
  misc_feature 1..935: note = BS_SP_CRTW, Brevundimonas SD212 (MBIC 03018), Yeast codon-optimized DNA sequence
  source 1..935: mol_type = genomic DNA
atgacagcagccgttgctgaacctagaatcgtcccaagacaaacttggatcggtttgaccttggccggtatgatcgttgccggttggggttccttgcacgtttacggtgtttactttcatcgttggggtacctcttccttagtcattgttccagctattgtcgctgttcaaacttggttgtccgttggtttgttcattgttgctcacgatgctatgcacggttctttggccccaggtagaccaagattgaacgctgctgttggtcgtttgaccttgggtttgtatgccggttttagatttgacagattgaagaccgctcaccacgcccaccacgctgccccaggtactgccgatgaccctgacttctacgctccagctccaagagctttcttgccatggtttttgaacttctttagaacctatttcggttggagagaaatggctgtcttgactgccttggttttaatcgctttattcggtttgggtgctagaccagctaacttattaaccttctgggccgctccagctttgttgtctgccttgcaattattcactttcggtacttggttgcctcacagacacactgaccaaccattcgctgacgctcaccatgccagatcttctggttacggtccagttttgtctttgttaacctgttttcacttcggtcgtcaccatgaacaccatttaactccatggcgtccatggtggagattgtggagaggtgaatcttaagagtatgcttctctttttttttgtaggccagtgataggaaagaacaatagaatataaatacgtcagaatataatagatatgtttttatatttagacctcgtacataggaataattgacgttttttttggccaacatttgaaatttttttttgttacctcgcgctgagcccaaacgggctccactacccgccgcggtcgcc

SEQ ID NO: 14 - DNA, 740 bp, Fulvimarina pelagi
  misc_feature 1..740: note = PELAGI_CRTZ. sterol desaturase family protein
  source 1..740: mol_type = genomic DNA
atgactatatggactctttactatgtatgtttgaccttggtcactatcggtttgatggaagtttacgcttggtgggctcataagttcattatgcacggtaaattcggttggggttggcacaaatcccatcacgaagaaactgaaggttggtttgaaaagaatgacttatacgctgttgttttcgctggttttgctatcgccttgttcatggttggtcacttcttgtccccaactttgttggccattgcttggggtatcactttatacggtttgttatacttcgttgctcatgacggtttggtccatcaaagatggccttttaactacgttccacacagaggttatgctaagcgtttagttcaagctcatagattacaccacgctgttgaaggtagagaacactgtgtctccttcggtttcttatacgctccacctattgaaaagttgaagcgtgatttgcgtgaatccggtattttagaacgtgaaagaatcgaacgttctttagaccaacaaggttctgctcacgccccagttagataagagtatgcttctctttttttttgtaggccagtgataggaaagaacaatagaatataaatacgtcagaatataatagatatgtttttatatttagacctcgtacataggaataattgacgttttttttggccaacatttgaaatttttttttgttacctcgcgctgagcccaaacgggctccactacccgccgcggtcgcc

SEQ ID NO: 15 - DNA, 1829 bp, unidentified
  misc_feature 1..1829: note = Yeast codon-optimized DNA sequence
  source 1..1829: mol_type = genomic DNA
atgattacaaccgatgtagtcattataggtgctggtcataacggtttggtctgcgctgcctacttattacaacgtggtttgggtgtcactttgttagaaaagagagaagttccaggtggtgctgctactaccgaagccttaatgcctgaattgtccccacaattcagatttaacagatgcgccatcgaccatgaattcattttcttaggtcctgtcttgcaagaattgaacttagctcaatacggtttggaatacttgttctgtgatccatccgttttctgtccaggtttagatggtcaagctttcatgtcttaccgttccttagaaaagacctgtgctcacattgctacttattccccaagagacgctgaaaagtaccgtcaatttgttaactactggactgacttgttaaatgccgttcaacctgcttttaacgctccaccacaagctttgttggacttggccttgaactacggttgggagaacttaaagtctgtcttagccatcgccggttccaagaccaaagctttggacttcattagaactatgattggttcccctgaagacgttttgaacgaatggttcgactctgaaagagtcaaagctccattagctagattgtgttccgaaattggtgcccctccttctcaaaagggttcttcttctggtatgatgatggtcgctatgagacacttagaaggtattgctcgtccaaaaggtggtactggtgctttgaccgaggccttggtcaaattggttcaagctcaaggtggtaagatcttgactgatcaaactgttaagcgtgtcttggtcgaaaacaaccaagctattggtgttgaagtcgccaacggtgaacaatacagagctaaaaaaggtgtcatttccaacattgatgcccgtcgtttgtttttacaattagtcgaaccaggtgctttagccaaagttaaccaaaacttgggtgaacgtttggaaagaagaaccgttaacaacaacgaagccattttgaagattgattgtgctttgtctggtttgcctcacttcactgctatggctggtccagaagatttgactggtactattttgattgctgactccgtccgtcacgtcgaagaagcccatgccttaattgctttgggtcaaattcctgatgctaacccatctttgtacttagatattccaactgttttggaccctactatggctcctccaggtcaacatactttgtggatcgagtttttcgccccttacagaatcgctggtttggaaggtaccggtttaatgggtactggttggactgatgaattgaaggaaaaggtcgctgatagagttatcgataaattgaccgactacgctccaaacttaaagtccttgatcatcggtagaagagtcgaatctccagccgaattagctcaacgtttaggttcttacaacggtaatgtttaccacttagacatgtctttagatcaaatgatgttcttgcgtccattaccagaaatcgccaactaccaaacccctatcaagaacttgtacttaaccggtgctggtactcatccaggtggttctatttctggtatgcctggtagaaactgtgccagagtcttcttaaagcaacaaagacgtttctggtaagagtatgcttctctttttttttgtaggccagtgataggaaagaacaatagaatataaatacgtcagaatataatagatatgtttttatatttagacctcgtacataggaataattgacgttttttttggccaacatttgaaatttttttttgttacctcgcgctgagcccaaacgggctccactacccgccgcggtcgcc

(19) United States
(12) Patent Application Publication
(10) Pub. No.: US 2026/0218213 A1
(43) Pub. Date: Jul. 30, 2026

(54) PRODUCTION OF CANTHAXANTHIN

(71) Applicant: AMYRIS, INC., Emeryville, CA (US)

(72) Inventors: Christopher J. PADDON, Emeryville, CA (US); Victor F. HOLMES, Emeryville, CA (US); Matthew E. JONES, Emeryville, CA (US); Yoseph TSEGAYE, Emeryville, CA (US)

(21) Appl. No.: 19/145,846

(22) PCT Filed: Jan. 10, 2024
(86) PCT No.: PCT/US2024/010960

§ 371 Date: Jul. 3, 2025

Related U.S. Application Data
(60) Provisional application No. 63/479,246, filed on Jan. 10, 2023.

(51) Int. Cl. C12N 15/81; C12N 9/02; C12N 9/10; C12N 9/90; C12N 15/52; C12P 23/00; C12R 1/865

(52) U.S. Cl. CPC C12N 15/81; C12N 9/001; C12N 9/0083; C12N 9/1085; C12N 9/90; C12N 15/52; C12P 23/00; C12R 2001/865; C12Y 114/99; C12Y 205/0101; C12Y 205/01029; C12Y 205/01032; C12Y 505/01019

(57) ABSTRACT

Provided herein are compositions and methods for production and recovery of canthaxanthin from a recombinant cell.

### 1. CROSS-REFERENCE TO RELATED APPLICATIONS

The present application claims the benefit of U.S. Provisional application Ser. No. 63/479,246, filed Jan. 10, 2023, the contents of which is hereby incorporated by reference in its entirety.

### 2. SEQUENCE LISTING

The application contains a Sequence Listing which has been submitted electronically in .XML format and is hereby incorporated by reference in its entirety. Said .XML copy, created on Jan. 9, 2024, is named “107345.00934.xml” and is 35,424 bytes in size. The sequence listing contained in this .XML file is part of the specification and is hereby incorporated by reference herein in its entirety.

### 3. FIELD OF THE INVENTION

Provided herein are compositions and methods useful for production of canthaxanthin from microbial cells.

### 4. BACKGROUND OF THE INVENTION

Isoprenoids are ubiquitous in nature. They comprise a diverse family of over 40,000 individual products, many of which are vital to living organisms. Isoprenoids serve to maintain cellular fluidity, electron transport, and other metabolic functions. A vast number of natural and synthetic isoprenoids have many applications, such as pharmaceuticals, cosmetics, perfumes, pigments and colorants, fungicides, antiseptics, nutraceuticals, and fine chemical intermediates.

Useful isoprenoids include carotenoids. Carotenoids are distributed in fish, animals, and crustaceans. The reddish orange carotenoid astaxanthin has been used as a pigmentation source in industry, for example, for food coloring or for trout, salmon, or poultry feed.

Traditionally, isoprenoids have been manufactured by extraction from natural sources such as plants, microbes, and animals. However, the yield by way of extraction is usually very low due to a number of profound limitations. First, most isoprenoids accumulate in nature in only small amounts. Second, the source organisms in general are not amenable to the large-scale cultivation that is necessary to produce commercially viable quantities of a desired isoprenoids.

Advances have been made in in the field of synthetic biology, and currently a number of isoprenoids are being produced at an industrial scale. Nevertheless, given the very large quantities of isoprenoid products needed for many commercial applications, there remains a need to improve systems and fermentation procedures that can produce isoprenoids more efficiently than available with current technologies. Also, efficient production of carotenoids, such as canthaxanthin, from renewable source is desirable.

Embodiments of the present invention meet these and other needs.

### 5. SUMMARY

Provided herein are genetically modified host cells, compositions, and methods for the improved production of canthaxanthin. These compositions and methods are based in part on the expression of certain gene products in host cells that have been genetically modified to produce canthaxanthin. Certain expressed gene products include enzymes that oxidize β-carotene. While not intending to be bound by any particular theory of operation, the examples herein demonstrate that expression of one or more β-carotene ketolase enzymes enhances oxidation of β-carotene and at least one intermediate useful for production of canthaxanthin. Expression of the one or more β-carotene ketolase enzymes improves the yield and productivity of canthaxanthin production. In addition, such expression is also shown to reduce accumulation of an intermediate and increase flux toward the production of canthaxanthin from β-carotene.

In one aspect, provided herein are genetically modified host cell capable of producing canthaxanthin, comprising nucleic acid capable of expressing a β-carotene ketolase selected from the group consisting of Rhizobium sp. Leaf321 β-carotene ketolase, Xanthobacter sp. β-carotene ketolase, Parvularcula oceani β-carotene ketolase, Erythrobacter luteus β-carotene ketolase, Fulvimarina pelagi β-carotene ketolase, Sphingomonas melonis β-carotene ketolase, Sphingomonas guangdonggensis β-carotene ketolase, Phenylbacterium deserti β-carotene ketolase, Brevundimonas sp. β-carotene ketolase, Brevudimonas vesicularis β-carotene ketolase, Hyphomicrobium sp. β-carotene ketolase, Bradyrhizobium sp. ORS 278 β-carotene ketolase, Paracoccus sp. β-carotene ketolase, Paracoccus haeundaensis β-carotene ketolase, Nostoc sp. PCC 7120 β-carotene ketolase, Haematococcus pluvaialis β-carotene ketolase, Agrobacterium radiobacter K84 β-carotene ketolase, Synechocystis sp. PCC6803 β-carotene ketolase. Nocardia farcinica IFM 10152 β-carotene ketolase, Mycobacterium smegmatis str. MC2 155 β-carotene ketolase, Arthrospira platensis NIES-39 β-carotene ketolase, Flavobacteria bacterium MS024-3C β-carotene ketolase, Caenorhabditis briggsae β-carotene ketolase, Fulvimarina pelagi β-carotene ketolase, Prochlorococcus marinus subsp. pastoris str. CCMP1986 β-carotene ketolase; Myxococcus xanthus DK 1622 (Q1D6N4); Myxococcus xanthus DK 1622 (Q1D169); and Bacillus clausii KSM-K16. In particular embodiments, the genetically modified host cells comprise Bradyrhizobium sp. β-carotene ketolase. In particular embodiments, the genetically modified host cells comprise Bradyrhizobium sp. β-carotene ketolase and Paracoccus sp. β-carotene ketolase. In additional embodiments, the genetically modified host cells further comprise enzymes sufficient to produce canthaxanthin. Each feature is described in detail below.

In another aspect, provided herein is a method for producing canthaxanthin comprising: culturing a population of the host cells of the invention in a medium with a carbon source under conditions suitable for making canthaxanthin to yield a culture broth; and recovering the canthaxanthin from the culture broth.

The compositions and methods are useful for producing canthaxanthin for any purpose, including as pigments or colorings. They are also useful for producing any compound that can be synthesized or biosynthesized from canthaxanthin. The compounds can be produced synthetically, or biosynthetically with downstream enzymes or pathways, or a combination thereof.

### 6. BRIEF DESCRIPTION OF THE FIGURES

FIG. 1 provides a metabolic pathway for conversion of FPP to canthaxanthin showing genes, gene origins, and enzyme names.

FIG. 2 provides relative amounts of C40-carotenoid pathway enzymes (N=4 replicates). The relative protein amount in the graph is the signal intensity of a given peptide normalized to signal intensity of an ACTI peptide.

FIG. 3 provides characterization of strains engineered with differential expression of the Paracoccus sp CrtW using different promoters in β-carotene co-producer strain AMR-13 in 96-well microtiter plates. (A) Production of β-carotene and canthaxanthin, average of 8 wells.

FIG. 4 provides characterization of strains engineered with differential expression of the Paracoccus sp CrtW using different promoters in β-carotene co-producer strain AMR-13 in 96-well microtiter plates. Production of farnesene. average and standard error of the mean for 8 replicates. Black indicates non-carotenoid producing starting strain, red indicates the β-carotene producing parent, green indicates other strains with successful performance, and grey indicates strains whose performance did not meet expectations.

FIG. 5 provides UV-UPLC chromatographic analysis of carotenoids and ketocarotenoids produced by AMR-23.

FIG. 6 provides identification by MS/MS fragmentation data analysis of the echinenone peak from cultured AMR-23 extract (FIG. 5) and echinenone standard.

FIG. 7 provides carotenoid production by fed-batch fermentation of strain AMR-23, including C40 chromatographic analysis of fermentation samples from strain AMR-23 at Day 4 and Day 7. The peak height ratio of echinenone to canthaxanthin increased from ~1:1 to ~3:1 between Day 4 and Day 7.

FIG. 8 provides production of β-carotene, echinenone, and canthaxanthin by strains expressing various combinations of genes encoding β-carotene ketolase enzymes. Genes were expressed from different strength promoters.

FIG. 9 provides production of β-carotene, echinenone and canthaxanthin by the strains described in FIG. 8. The table below the graphs shows promoter strengths (relative to pGAL1) of the promoters used to express the indicated genes.

FIG. 10 provides C40 chromatographic analysis of bioreactor fed batch fermentation samples of AMR-23 (Paracoccus sp. CrtW; upper panel) and AMR-27 (Bradyrhizobium sp. CrtW; lower pancl) at Day 4. Peak area ratio of canthaxanthin to cchinenone is higher for strain AMR-27 (lower panel) that expresses the CrtW variant from Bradyrhizobium sp. strain ORS278.

FIG. 11 provides chromatographic analysis of fermentation samples of strain AMR-28 at Day 4 and Day 7. Strain AMR-28 expresses the CrtW variants from both Bradyrhizobium sp. strain ORS278 and Paracoccus species.

### 7. DETAILED DESCRIPTION OF THE EMBODIMENTS

### 7.1 Definitions

As used herein, the term “about” refers to a reasonable range about a value as determined by the practitioner of skill. In certain embodiments, the term about refers to ±one, two, or three standard deviations. In certain embodiments, the term about refers to ±5%, 10%, 20%, or 25%. In certain embodiments, the term about refers to ±0.1, 0.2, or 0.3 logarithmic units, e.g. pH units.

The terms “isoprenoid,” “isoprenoid compound,” “terpene,” “terpene compound,” “terpenoid,” and “terpenoid compound” are used interchangeably herein, and refer to any compound that is capable of being derived from isopentenyl pyrophosphate (IPP). The number of C-atoms present in the isoprenoids is typically evenly divisible by five (e.g., C5, C10, C15, C20, C25, C30 and C40). Irregular isoprenoids and polyterpenes have been reported, and are also included in the definition of “isoprenoid.” Isoprenoid compounds include, but are not limited to, monoterpenes, diterpenes, triterpenes, sesquiterpenes, and polyterpenes.

As used herein, the term “prenyl diphosphate” is used interchangeably with “prenyl pyrophosphate,” and includes monoprenyl diphosphates having a single prenyl group (e.g., IPP and DMAPP), as well as polyprenyl diphosphates that include 2 or more prenyl groups. Monoprenyl diphosphates include isopentenyl pyrophosphate (IPP) and its isomer dimethylallyl pyrophosphate (DMAPP).

As used herein, the term “terpene synthase” refers to any enzyme that enzymatically modifies IPP, DMAPP, or a polyprenyl pyrophosphate, such that a terpenoid precursor compound is produced. The term “terpene synthase” includes enzymes that catalyze the conversion of a prenyl diphosphate into an isoprenoid or isoprenoid precursor.

The word “pyrophosphate” is used interchangeably herein with “diphosphate” and refers to two phosphate groups covalently bonded. Thus, e.g., the terms “prenyl diphosphate” and “prenyl pyrophosphate” are interchangeable; the terms “isopentenyl pyrophosphate” and “isopentenyl diphosphate” are interchangeable; the terms farnesyl diphosphate” and farnesyl pyrophosphate” are interchangeable; etc.

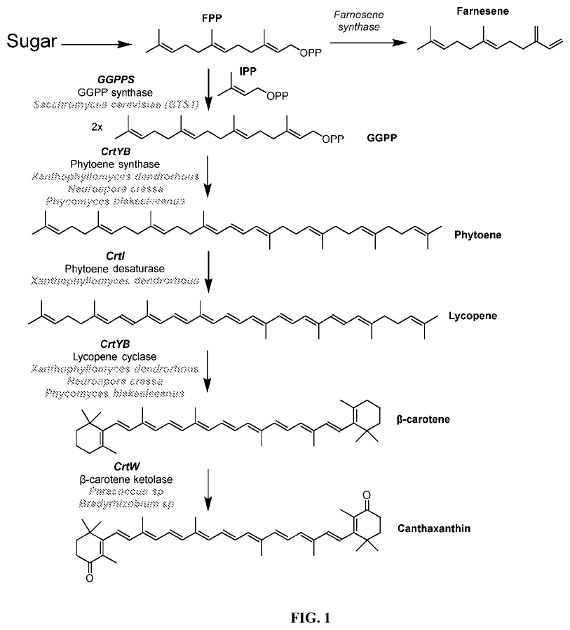

The term “mevalonate pathway” or “MEV pathway” is used herein to refer to the biosynthetic pathway that converts acetyl-CoA to IPP. The mevalonate pathway comprises enzymes that catalyze the following steps: (a) condensing two molecules of acetyl-CoA to acetoacetyl-CoA (e.g., by action of acetoacetyl-CoA thiolase); (b) condensing acetoacetyl-CoA with acetyl-CoA to form hydroxymethylglutaryl-CoenzymeA (HMG-COA) (e.g., by action of HMG-COA synthase (HMGS)); (c) converting HMG-COA to mevalonate (e.g., by action of HMG-COA reductase (HMGR)); (d) phosphorylating mevalonate to mevalonate 5-phosphate (e.g., by action of mevalonate kinase (MK)); (e) converting mevalonate 5-phosphate to mevalonate 5-pyrophosphate (e.g., by action of phosphomevalonate kinase (PMK)); and (f) converting mevalonate 5-pyrophosphate to isopentenyl pyrophosphate (e.g., by action of mevalonate pyrophosphate decarboxylase (MPD)). The mevalonate pathway is illustrated schematically in FIG. 1. The “top half” of the mevalonate pathway refers to the enzymes responsible for the conversion of acetyl-CoA to mevalonate.

The term “I-deoxy-D-xylulose 5-diphosphate pathway” or “DXP pathway” is used herein to refer to the pathway that converts glyceraldehyde-3-phosphate and pyruvate to IPP and DMAPP through a DXP pathway intermediate, where DXP pathway comprises enzymes that catalyze the reaction. Typical enzymes of the DXP pathway include DXS, DXR, CMS, CMK, MCS, HDS, and HDR. Dxs is 1-deoxy-D-xylulose-5-phosphate synthase; Dxr is 1-deoxy-D-xylulose-5-phosphate reductoisomerase (also known as IspC); IspD (CMS) is 4-diphosphocytidyl-2C-methyl-D-erythritol synthase; IspE (CMK) is 4-diphosphocytidyl-2C-methyl-D-erythritol synthase; IspF (MCS) is 2C-methyl-D-erythritol 2,4-cyclodiphosphate synthase; IspG (HDS) is 1-hydroxy-2-methyl-2-(E)-butenyl 4-diphosphate synthase; and IspH (HDR) is isopentenyl/dimethylallyl diphosphate synthase.

As used herein, the term “prenyl transferase” is used interchangeably with the terms “isoprenyl diphosphate synthase” and “polyprenyl synthase” (e.g., “GPP synthase,” “FPP synthase.” “OPP synthase,” etc.) to refer to an enzyme that catalyzes the consecutive 1′-4 condensation of isopentenyl diphosphate with allylic primer substrates, resulting in the formation of prenyl diphosphates of various chain lengths.

As used herein, the term “target compound(s)” refers to compounds to be recovered from a host cell genetically modified with one or more heterologous nucleic acids encoding enzymes of a biosynthetic pathway for producing the target compound(s), and does not include metabolites which may be incidentally produced during the production of the target compound(s). In particular embodiments, two or more target compounds are recovered from a culture.

The term “fermentation run” refers to one complete cycle of a batch, semi-continuous or continuous fermentation. A fermentation run preferably begins when the fermentor is initially filled with starting materials and is inoculated with the proper organisms. A fermentation run preferably ends when the fermentor organisms are no longer active, or when the fermentor is emptied.

The term “inoculation” refers to the placement of host cells (e.g., genetically modified microbial cells) that will grow to form the microbial culture placed in a culture medium, such as a fermentation tank comprising media to be fermented.

The term “single inoculum” refers to the material used in an inoculation, for example, a composition comprising host cells (e.g., genetically modified microbial cells) placed in a culture medium, such as a fermentation tank comprising media, at an initial time point to grow biomass.

The term “co-production” refers to producing two or more target compounds from a single inoculum, i.e., from cells produced from a single host cell. As used herein, the term, co-production can refer to concurrent or simultaneous production of two or more compounds in a single fermentation run in a fermentor. The term, co-production, can also refer to a sequential production of two or more target compounds from a single inoculum, wherein at least one target compound produced by activating expression of enzymes of a biosynthetic pathway for the target compound in a first fermentation run, followed by activating expression of enzymes of another biosynthetic pathway for another target compound. In certain embodiments, a sequential production can be achieved in two separate fermentation runs.

As used herein, the term “carotenoid” refers to a class of hydrocarbons having a conjugated polyene carbon skeleton formally derived from isoprene. This class of molecules is composed of triterpenes and tetraterpenes and their oxygenated derivatives; and, these molecules typically have strong light absorbing properties and impart color. Carotenoids can be acyclic or terminated with one (monocyclic) or two (bicyclic) cyclic end groups. The term “carotenoid” may include both carotenes and xanthophylls. Carotenoids that are particularly suitable in the present description are monocyclic and bicyclic carotenoids. The term “carotenoid” may include both carotenes and xanthophylls. A “carotene” refers to a hydrocarbon carotenoid (e.g., β-carotene and lycopene). In contrast, the term “xanthophyll” refers to a C40 carotenoid that contains one or more oxygen atoms in the form of hydroxy-, methoxy-, oxo-, epoxy-, carboxy-, or aldehydic functional groups. Examples of xanthophylls include, but are not limited to antheraxanthin, adonixanthin, astaxanthin (i.e., 3,3′-dihydroxy-β,β-carotene-4,4′-dione), canthaxanthin (i.e., β,β-carotene-4,4′-dione), β-cryptoxanthin, keto-γ-carotene, echinenone, 3-hydroxyechinenone, 3′-hydroxyechinenone, zeaxanthin, adonirubin, tetrahydroxy-β,β′-caroten-4,4′-dione, tetrahydroxy-β,β′-caroten-4-one, caloxanthin, erythroxanthin, nostoxanthin, flexixanthin, 3-hydroxy-γ-carotene, 3-hydroxy-4-keto-γ-carotene, bacteriorubixanthin, bacteriorubixanthinal and lutein.

As used herein, gene names are typically capitalized and italicized, e.g. BTS1. Protein names are typically initially capitalized and not italicized, e.g. Bts1 or Bts1p. However, where the term protein is indicated, then the protein is intended. For instance, those of skill will recognize that “BTS1 protein” is intended to refer to Bts1p.

As used herein, the term “genetic switch” refers to one or more genetic elements that allows controlled expression enzymes that produce the first isoprenoid compound and enzymes that produce the second isoprenoid compound. In a first configuration, the genetic switch could promote expression of enzymes that produce the first isoprenoid compound and suppress enzymes that produce the second isoprenoid compound. In a second configuration, the genetic switch could suppress expression of enzymes that produce the first isoprenoid compound and promote enzymes that produce the second isoprenoid compound. In a third configuration, the genetic switch could promote expression of enzymes that produce the first isoprenoid compound and promote enzymes that produce the second isoprenoid compound. In a fourth configuration, the genetic switch could suppress expression of enzymes that produce the first isoprenoid compound and suppress enzymes that produce the second isoprenoid compound. For example, a genetic switch can include one or more promoters operably linked to one or more genes encoding a biosynthetic enzyme or one or more promoters operably linked to a transcriptional regulator which regulates expression one or more biosynthetic enzymes.

As used herein, the term “parent cell” refers to a cell that has an identical genetic background as a genetically modified host cell disclosed herein except that it does not comprise one or more particular genetic modifications engineered into the modified host cell, for example, one or more modifications selected from the group consisting of: heterologous expression of an enzyme of a carotenoid pathway, or heterologous expression of BTS1, CrtYB, CrtI, or CrtW.

As used herein, the term “naturally occurring” refers to what is found in nature. For example, gene product that is present in an organism that can be isolated from a source in nature and that has not been intentionally modified by a human in the laboratory is naturally occurring gene product. Conversely, as used herein, the term “non-naturally occurring” refers to what is not found in nature but is created by human intervention. In certain embodiments, naturally occurring genomic sequences are modified, e.g., codon optimized, for use in the organisms provided herein.

As used herein, the term “heterologous” refers to what is not normally found in nature. The term “heterologous nucleotide sequence” refers to a nucleotide sequence not normally found in a given cell in nature. As such, a heterologous nucleotide sequence may be: (a) foreign to its host cell (i.e., is “exogenous” to the cell); (b) naturally found in the host cell (i.e., “endogenous”) but present at an unnatural quantity in the cell (i.e., greater or lesser quantity than naturally found in the host cell); or (c) be naturally found in the host cell but positioned outside of its natural locus. The term “heterologous enzyme” refers to an enzyme that is not normally found in a given cell in nature. The term encompasses an enzyme that is: (a) exogenous to a given cell (i.e., encoded by a nucleotide sequence that is not naturally present in the host cell or not naturally present in a given context in the host cell); and (b) naturally found in the host cell (e.g., the enzyme is encoded by a nucleotide sequence that is endogenous to the cell) but that is produced in an unnatural amount (e.g., greater or lesser than that naturally found) in the host cell.

On the other hand, the term “native” or “endogenous” as used herein with reference to molecules, and in particular enzymes and nucleic acids, indicates molecules that are expressed in the organism in which they originated or are found in nature, independently of the level of expression that can be lower, equal, or higher than the level of expression of the molecule in the native microorganism. It is understood that expression of native enzymes or polynucleotides may be modified in recombinant microorganisms.

As used herein, the term “production” generally refers to an amount of isoprenoid or produced by a genetically modified host cell provided herein. In some embodiments, production is expressed as a yield of isoprenoid by the host cell. In other embodiments, production is expressed as a productivity of the host cell in producing the isoprenoid.

As used herein, the term “productivity” refers to production of an isoprenoid by a host cell, expressed as the amount of isoprenoid produced (by weight) per amount of fermentation broth in which the host cell is cultured (by volume) over time (per hour).

As used herein, the term “yield” refers to production of an isoprenoid by a host cell, expressed as the amount of isoprenoid produced per amount of carbon source consumed by the host cell, by weight.

As used herein, the term “variant” refers to a polypeptide differing from a specifically recited “reference” polypeptide (e.g., a wild-type sequence) by amino acid insertions, deletions, mutations, and substitutions, but retains an activity that is substantially similar to the reference polypeptide. In some embodiments, the variant is created by recombinant DNA techniques, such as mutagenesis. In some embodiments, a variant polypeptide differs from its reference polypeptide by the substitution of one basic residue for another (i.e., Arg for Lys), the substitution of one hydrophobic residue for another (i.e., Leu for Ile), or the substitution of one aromatic residue for another (i.e., Phe for Tyr), etc. In some embodiments, variants include analogs wherein conservative substitutions resulting in a substantial structural analogy of the reference sequence are obtained. Examples of such conservative substitutions, without limitation, include glutamic acid for aspartic acid and vice-versa; glutamine for asparagine and vice-versa; serine for threonine and vice-versa; lysine for arginine and vice-versa; or any of isoleucine, valine, or leucine for each other.

As used herein, the term “sequence identity” or “percent identity,” in the context or two or more nucleic acid or protein sequences, refer to two or more sequences or subsequences that are the same or have a specified percentage of amino acid residues or nucleotides that are the same. For example, the sequence can have a percent identity of at least 60%, at least 65%, at least 70%, at least 75%, at least 80%, at least 85%, at least 90%, at least 91% at least 92%, at least 93%, at least 94%, at least 95%, at least 96%, at least 97%, at least 98%, at least 99%, or higher identity over a specified region to a reference sequence when compared and aligned for maximum correspondence over a comparison window, or designated region as measured using a sequence comparison algorithm or by manual alignment and visual inspection. For example, percent of identity is determined by calculating the ratio of the number of identical nucleotides (or amino acid residues) in the sequence divided by the length of the total nucleotides (or amino acid residues) minus the lengths of any gaps.

For convenience, the extent of identity between two sequences can be ascertained using computer program and mathematical algorithms known in the art. Such algorithms that calculate percent sequence identity generally account for sequence gaps and mismatches over the comparison region. Programs that compare and align sequences, like Clustal W (Thompson et al., (1994) Nucleic Acids Res., 22:4673-4680), ALIGN (Myers et al., (1988) CABIOS, 4:11-17), FASTA (Pearson et al., (1988) PNAS, 85:2444-2448; Pearson (1990), Methods Enzymol., 183:63-98) and gapped BLAST (Altschul et al., (1997) Nucleic Acids Res., 25:3389-3402) are useful for this purpose. The BLAST or BLAST 2.0 (Altschul et al., J. Mol. Biol. 215:403-10, 1990) is available from several sources, including the National Center for Biological Information (NCBI) and on the Internet, for use in connection with the sequence analysis programs BLASTP, BLASTN, BLASTX, TBLASTN, and TBLASTX. Additional information can be found at the NCBI web site.

In certain embodiments, the sequence alignments and percent identity calculations can be determined using the BLAST program using its standard, default parameters. For nucleotide sequence alignment and sequence identity calculations, the BLASTN program is used with its default parameters (Gap opening penalty=5, Gap extension penalty=2, Nucleic match=1, Nucleic mismatch=−3, Expectation value=10.0, Word size=11). For polypeptide sequence alignment and sequence identity calculations, BLASTP program is used with its default parameters (Alignment matrix=BLOSUM62; Gap costs: Existence=11, Extension=1; Compositional adjustments-Conditional compositional score, matrix adjustment; Expectation value=10.0; Word size=6; Max matches in a query range=0). Alternatively, the following program and parameters are used: Align Plus software of Clone Manager Suite, version 5 (Sci-Ed Software); DNA comparison: Global comparison, Standard Linear Scoring matrix, Mismatch penalty=2, Open gap penalty=4, Extend gap penalty=1. Amino acid comparison: Global comparison, BLOSUM 62 Scoring matrix.

### 7.2 Description

In one aspect, provided herein are nucleic acids, expression vectors, and host cells which express one or more enzymes useful for the production of canthaxanthin. The enzymes are described in detail herein. In certain embodiments, the host cells can produce canthaxanthin from a carbon source in a culture medium. In certain embodiments, the host cells provide improved yield and/or productivity compared to a parent strain. In certain embodiments, the host cells provide byproducts, intermediates, and/or side products, e.g., echinenone, compared to a parent strain.

Carotenoids are red, yellow, and orange pigments that are widely distributed in nature. C₄₀ carotenoids belong to the category of tetraterpenes (i.e., they have 40 carbon atoms, being built from four terpene units each containing 10 carbon atoms). There are two general classes of carotenoids: carotenes and xanthophylls. Carotenes consist only of carbon and hydrogen atoms. Xanthopylls have one or more oxygen atoms. Hydrocarbon carotenoids are classified as carotenes while those containing oxygen are known as xanthopylls. Canthaxanthin is an oxidized carotenoid also known as a cantaxanthin, cantaxanthine, and canthaxanthine, with the IUPAC name β,β-Carotene-4,4′-dione (i.e., a carotenoid with two ketone groups).

Farnesene (or other sesquiterpenes) and carotenoids, including canthaxanthin, are isoprenoids that can be produced in yeast via the mevalonate pathway. The enzymes of the mevalonate pathway (from acetyl-CoA to IPP) are further described herein. In addition, the DXP pathway can be used to produce isoprenoid precursors.

The over-expression of the mevalonate pathway in yeast (Saccharomyces cerevisiae) has been described in the scientific literature (e.g., Notman et al., J. Am. Chem. Soc., 128, 2006, 13982-13983; He et al., Mol. Membr. Biol. 3-4, 2012, 107-113). In certain embodiments, one or more enzymes of the mevalonate pathway are over-expressed as previously described.

Production of β-carotene in engineered S. cerevisiae has been described. Kim et al., Food Sci. Biotechnol. 19, 2010, 263-266. The β-carotene biosynthetic pathway is reproduced as FIG. 1. As illustrated in the exemplary β-carotene biosynthetic pathway shown in FIG. 1, after IPP is formed from the mevalonate pathway, it can be converted into dimethylallyl pyrophosphate (DMAPP) by an IPP isomerase. IPP and DMAPP are condensed by geranyl pyrophosphate synthase (GPPS) to produce geranyl pyrophosphate (GPP). GPP and IPP can be combined by farnesyl pyrophosphate synthase (FPPS) to produce farnesyl pyrophosphate (FPP). FPP and IPP can be combined by a geranyl pyrophosphate synthase (GGPP synthase) to produce GGPP. Exemplary nucleic acids that encode GGPP synthases include BTS1 gene (S. cerevisiae) and CrtE gene (X. dendrorhous). GGPP and GGPP can be combined by phytoene synthase (encoded by CrtB) to produce phytoene. In the exemplary β-carotene biosynthetic pathway shown in FIG. 1, a bifunctional enzyme (phytoene synthase/lycopene cyclase) encoded by CrtYB is shown for this enzymatic reaction step. Lycopene can be converted to β-carotene by the enzymatic action of a lycopene cyclase (encoded by CrtB gene). In the exemplary β-carotene biosynthetic pathway shown in FIG. 1, a bifunctional enzyme (phytoene synthase/lycopene cyclase) encoded by CrtYB gene is shown for this enzymatic reaction step. It is noted that phytoene shown in FIG. 1 does not impart any color, and therefore is not considered a carotenoid, whereas lycopene and β-carotene are considered as carotenoids.

The production of canthaxanthin starting from β-carotene is shown in FIG. 1. The arrows indicate the reactions catalyzed by the enzymes encoded by BTS1, CrtYB, CrtI, CrtYB, and CrtW, respectively. β-carotene is converted to canthaxanthin in steps in which two keto are added to each ring by β-carotene ketolase encoded by crtW. A single X. dendrorhous gene, CrtYB, encodes a bifunctional enzyme phytoene synthase/lycopene cyclase. See Verdoes et al., 1999, Mol. Gen. Genet. 262:453-461.

In certain embodiments, any suitable nucleic acid(s) encoding enzymes in the β-carotene/canthaxanthin biosynthetic pathway can be used in producing genetically modified host cells or methods for co-production of one or more carotenoids with another isoprenoid. The exemplary nucleic acids encoding such enzymes useful in present embodiments are shown in Table 1 below. Additional nucleic acids useful in the production of beta-carotene/canthaxanthin pathways are further described in Section 5.6 below.

TABLE 1
List of exemplary nucleic acids and encoded enzymes suitable for enzymatic reactions shown in FIG. 1.

| Gene | | | | | |
|---|---|---|---|---|---|
| Name | Enzyme Activity | Organism | GenBank ID | UniProt | Reference |
| BTS1 | GGPP Synthase | S. cerevisiae | | | Jiang et al., 1995, |
| | | | | | J. Biol. Chem. |
| | | | | | 270(37): 21793-9. |
| CrtYB | Phytoene | Xanthophyllomyces | | | Verwaal et al., |
| | synthase/Lycopene | dendrorhous | | | 2007, App. |
| | cyclase | | | | Environ. Microbiol. |
| | | | | | 73(13): 4342-50 |
| | | | | | (2007) |
| CrtI | Phytoene | Xanthophyllomyces | | | Verwaal et al |
| | desaturase | dendrorhous | | | (2007) |
| CrtW | β-Carotene | Paracoccus sp. Strain | BAE47465.1 | CRTW_PARSN | Ukibe et al, 2009, |
| | Ketolase | N81106 | | | Appl. Environ. |
| | | (Agrobacterium | | | Microbiol. 75(22): |
| | | aurantiacum) | | | 7205-7211 |

### 7.2.1 Host Cells

In one aspect, provided herein are nucleic acids, expression vectors, and host cells which express one or more enzymes useful for the production of canthaxanthin. In certain embodiments, the host cells can produce canthaxanthin from a carbon source in a culture medium. In certain embodiments, the host cells provide improved yield and/or productivity compared to a parent strain. In certain embodiments, the host cells provide byproducts, intermediates, and/or side products, e.g., echinenone, compared to a parent strain. In advantageous embodiments, the host cell comprises one or more enzymatic pathways capable of making canthaxanthin, said pathways taken individually or together.

In certain embodiments, host cells according to the embodiments herein produce at least 5%, at least 10%, at least 15%, at least 20%, or at least 25% more total canthaxanthin compared to a parent strain. In certain embodiments, host cells according to the embodiments herein produce 2-fold, 3-fold, 4-fold, 5-fold, or 10-fold less echinenone compared to a parent strain. In certain embodiments, the percent increases are with respect to canthaxanthin titer (g/L). In certain embodiments, the percent increases are with respect to canthaxanthin yield (weight %). In certain embodiments, the percent increases are with respect to canthaxanthin productivity (g/L/h). In certain embodiments, the percent increases are with respect to canthaxanthin total mass produced (g). In certain embodiments, host cells according to the embodiments herein produce increased canthaxanthin, and produce less echinenone, compared to a parent strain.

In one aspect, provided herein are genetically modified host cell capable of producing canthaxanthin comprising a nucleic acid capable of expressing Bradyrhizobium β-carotene ketolase. In certain embodiments, the nucleic acid is according to SEQ ID NO:1, or a variant thereof. In certain embodiments, the nucleic acid is according to SEQ ID NO:1, or a variant thereof having at least, 85, 90, 95, 99, or 100% sequence identity to SEQ ID NO:1. In certain embodiments, the nucleic acid encodes Bradyrhizobium β-carotene ketolase protein, or a variant thereof. In certain embodiments, the nucleic acid encodes Bradyrhizobium β-carotene ketolase protein, or a variant thereof having at least 80, 85, 90, 95, 99, or 100% sequence identity to SEQ ID NO:2. In certain embodiments, the genetically modified host cell comprises Bradyrhizobium β-carotene ketolase protein, or a variant thereof. In certain embodiments, the host cell comprises Bradyrhizobium β-carotene ketolase protein, or a variant thereof having at least 80, 85, 90, 95, 99, or 100% sequence identity to SEQ ID NO:2.

In certain embodiments, the Bradyrhizobium β-carotene ketolase is expressed in the genetically modified host cell. In certain embodiments, the Bradyrhizobium β-carotene ketolase is overexpressed in the genetically modified host cell. In certain embodiments, the Bradyrhizobium β-carotene ketolase is expressed from a GAL promoter in the genetically modified host cell. In certain embodiments, the Bradyrhizobium β-carotene ketolase is expressed from an inducible GAL promoter in the genetically modified host cell, as described herein.

In certain embodiments, the genetically modified host cell expresses one or more additional enzymes useful for the production of canthaxanthin. The additional enzymes can be any additional enzymes deemed useful by the person of skill.

In certain embodiments, the genetically modified host cell comprises one or more additional β-carotene ketolase enzymes. While not intending to be bound by any particular theory of operation, the examples herein demonstrate that a pair of β-carotene ketolase can complement each other to facilitate the two oxidations of β-carotene to canthaxanthin. In certain embodiments, the genetically modified host cell further comprises a nucleic acid capable of expressing Paracoccus β-carotene ketolase, or a variant thereof. In certain embodiments, the genetically modified host cell further comprises a nucleic acid capable of expressing Paracoccus β-carotene ketolase, or a variant thereof having at least 80, 85, 90, 95, 99, or 100% sequence identity to SEQ ID NO:3. In certain embodiments, the nucleic acid encodes Paracoccus β-carotene ketolase protein, or a variant thereof. In certain embodiments, the nucleic acid encodes Paracoccus β-carotene ketolase protein, or a variant thereof having at least 80, 85, 90, 95, 99, or 100% sequence identity to SEQ ID NO:4. In certain embodiments, the genetically modified host cell further comprises Paracoccus β-carotene ketolase, or a variant thereof. In certain embodiments, the genetically modified host cell further comprises Paracoccus β-carotene ketolase, or a variant thereof, having at least 80, 85, 90, 95, 99, or 100% sequence identity to SEQ ID NO:4.

In certain embodiments, the genetically modified host cell further comprises a nucleic acid capable of expressing Bts1p, or a variant thereof. In certain embodiments, the genetically modified host cell further comprises a nucleic acid capable of expressing Bts1p, or a variant thereof having at least 80, 85, 90, 95, 99, or 100% sequence identity to SEQ ID NO:5. In certain embodiments, the nucleic acid encodes Bts1p, or a variant thereof. In certain embodiments, the nucleic acid encodes Bts1p, or a variant thereof having at least 80, 85, 90, 95, 99, or 100% sequence identity to SEQ ID NO:6. In certain embodiments, the genetically modified host cell further comprises Bts1p, or a variant thereof. In certain embodiments, the genetically modified host cell further comprises Bts1p, or a variant thereof, having at least 80, 85, 90, 95, 99, or 100% sequence identity to SEQ ID NO:6.

In certain embodiments, the genetically modified host cell further comprises a nucleic acid capable of expressing phytoene synthase, or a variant thereof. In certain embodiments, the genetically modified host cell further comprises a nucleic acid capable of expressing phytoene synthase, or a variant thereof having at least 80, 85, 90, 95, 99, or 100% sequence identity to SEQ ID NO:7. In certain embodiments, the nucleic acid encodes phytoene synthase, or a variant thereof. In certain embodiments, the nucleic acid encodes phytoene synthase, or a variant thereof having at least 80, 85, 90, 95, 99, or 100% sequence identity to SEQ ID NO:8. In certain embodiments, the genetically modified host cell further comprises phytoene synthase, or a variant thereof. In certain embodiments, the genetically modified host cell further comprises phytoene synthase, or a variant thereof, having at least 80, 85, 90, 95, 99, or 100% sequence identity to SEQ ID NO:8.

In certain embodiments, the genetically modified host cell further comprises a nucleic acid capable of expressing phytoene desaturase, or a variant thereof. In certain embodiments, the genetically modified host cell further comprises a nucleic acid capable of expressing phytoene desaturase, or a variant thereof having at least 80, 85, 90, 95, 99, or 100% sequence identity to SEQ ID NO:9. In certain embodiments, the genetically modified host cell further comprises phytoene desaturase, or a variant thereof. In certain embodiments, the nucleic acid encodes phytoene desaturase, or a variant thereof. In certain embodiments, the nucleic acid encodes phytoene desaturase, or a variant thereof having at least 80, 85, 90, 95, 99, or 100% sequence identity to SEQ ID NO:10. In certain embodiments, the genetically modified host cell further comprises phytoene desaturase, or a variant thereof, having at least 80, 85, 90, 95, 99, or 100% sequence identity to SEQ ID NO:10.

In certain embodiments, the genetically modified host cell further comprises a nucleic acid capable of expressing lycopene cyclase, or a variant thereof. In advantageous embodiments, the phytoene synthase and the lycopene cyclase activities are found in the same bifunctional enzyme. In certain embodiments, the genetically modified host cell further comprises a nucleic acid capable of expressing lycopene cyclase, or a variant thereof having at least 80, 85, 90, 95, 99, or 100% sequence identity to SEQ ID NO:7. In certain embodiments, the nucleic acid encodes lycopene cyclase, or a variant thereof. In certain embodiments, the nucleic acid encodes lycopene cyclase, or a variant thereof having at least 80, 85, 90, 95, 99, or 100% sequence identity to SEQ ID NO:8. In certain embodiments, the genetically modified host cell further comprises lycopene cyclase, or a variant thereof. In certain embodiments, the genetically modified host cell further comprises lycopene cyclase, or a variant thereof, having at least 80, 85, 90, 95, 99, or 100% sequence identity to SEQ ID NO:8.

In certain embodiments, the genetically modified host cells further comprise a GAS4 deletion. The GAS4 gene can be deleted according to techniques apparent to the person of skill, such as those provided in the examples herein.

Expression or overexpression can be according to any technique apparent to those of skill in the art. In certain embodiments, the genes are overexpressed from a promoter useful in the host cell. In certain embodiments, the genes are overexpressed from a S. cerevisiae promoter. In certain embodiments, the promoter is selected from the group consisting of pPGK1, pTDH3, pENO2, pADH1, pTPI1, pTEF1, pTEF2, pTEF3, pGAL1, pGAL2, pGAL7, pGAL10, GAL1, pRPL3, pRPL15A, pRPL4, pRPL8B, pSSA1, pSSB1, pCUP1, pTPS1, pHXT7, pADH2, pCYC1, and pPDA1. In certain embodiments, the genes are overexpressed from a GAL promoter. In certain embodiments, the genes are overexpressed from a promoter selected from the group consisting of pGAL1, pGAL2, pGAL7, pGAL10, and variants thereof.

In certain embodiments, one, some, or all of the heterologous promoters in the host cells are inducible. The inducible promoter system can be any recognized by those of skill in the art. In particular embodiments, the promoters are inducible by maltose. In an advantageous embodiment, the host cells comprise a GAL regulon that is inducible by maltose. Examples of the Gal regulon which are further repressed or induced by a maltose are described in PCT Application Publications WO2015/020649, WO2016/210343, and WO2016210350, each of which is incorporated by reference in its entirety. In certain embodiment, a maltose switchable strain is built on top of a non-switchable strain by chromosomally integrating a copy of GAL80 under the control of a maltose-responsive promoter such as pMAL32. In certain embodiments, the GAL80 gene product is mutated for temperature sensitivity, e.g. to facilitate further control. In certain embodiments, the GAL80 gene product is fused to a temperature-sensitive polypeptide. In certain embodiments, the GAL80 gene product is fused to a temperature-sensitive DHFR polypeptide or fragment. Additional description of switchable strains are found in U.S. Patent Application Publication No. US 2016/0177341 and PCT Application Publication No. WO 2016/210350, each of which is incorporated herein by reference in its entirety.

For each of the polypeptides and nucleic acids described above, the host cells can comprise variants thereof. In certain embodiments, the variant can comprise up to 15, 10, 9, 8, 7, 6, 5, 4, 3, 2, or 1 amino acid substitutions relative to the relevant polypeptide. In certain embodiments, the variant can comprise up to 15, 10, 9, 8, 7, 6, 5, 4, 3, 2, or 1 conservative amino acid substitutions relative to the reference polypeptide. In certain embodiments, any of the nucleic acids described herein can be optimized for the host cell, for instance codon optimized. Variants and optimization are described in detail below.

In certain embodiments, the additional enzymes are native, unless specified otherwise above. Native enzymes can be expressed from codon optimized nucleic acids. In advantageous embodiments, the additional enzymes are heterologous. In certain embodiments, two or more enzymes can be combined in one polypeptide.

In certain embodiments, the genetically modified host cell is genetically modified to overexpress one or more enzymes of the mevalonate pathway. In certain embodiments, the genetically modified host cell is genetically modified to overexpress, all of the enzymes of the mevalonate pathway.

### 7.2.2 Cell Strains

The host cells can be any cells deemed useful by those of skill. Host cells useful in the compositions and methods provided herein include archae, prokaryotic, or eukaryotic cells.

Suitable prokaryotic hosts include, but are not limited, to any of a variety of gram-positive, gram-negative, or gram-variable bacteria. Examples include, but are not limited to, cells belonging to the genera: Agrobacterium, Alicyclobacillus, Anabaena, Anacystis, Arthrobacter, Azobacter, Bacillus, Brevibacterium, Chromatium, Clostridium, Corynebacterium, Enterobacter, Erwinia, Escherichia, Lactobacillus, Lactococcus, Mesorhizobium, Methylobacterium, Microbacterium, Phormidium, Pseudomonas, Rhodobacter, Rhodopseudomonas, Rhodospirillum, Rhodococcus, Salmonella, Scenedesmun, Serratia, Shigella, Staphylococcus, Strepromyces, Synnecoccus, and Zymomonas. Examples of prokaryotic strains include, but are not limited to: Bacillus subtilis, Bacillus amyloliquefacines, Brevibacterium ammoniagenes, Brevibacterium immariophilum, Clostridium beigerinckii, Enterobacter sakazakii, Escherichia coli, Lactococcus lactis, Mesorhizobium loti, Pseudomonas aeruginosa, Pseudomonas mevalonii, Pseudomonas pudica, Rhodobacter capsulatus, Rhodobacter sphaeroides, Rhodospirillum rubrum, Salmonella enterica, Salmonella typhi, Salmonella typhimurium, Shigella dysenteriae, Shigella flexneri, Shigella sonnei, and Staphylococcus aureus. In a particular embodiment, the host cell is an Escherichia coli cell.

Suitable archae hosts include, but are not limited to, cells belonging to the genera: Aeropyrum, Archaeglobus, Halobacterium, Methanococcus, Methanobacterium, Pyrococcus, Sulfolobus, and Thermoplasma. Examples of archae strains include, but are not limited to: Archaeoglobus fulgidus, Halobacterium sp., Methanococcus jannaschii, Methanobacterium thermoautotrophicum, Thermoplasma acidophilum, Thermoplasma volcanium, Pyrococcus horikoshii, Pyrococcus abyssi, and Aeropyrum pernix.

Suitable eukaryotic hosts include, but are not limited to, fungal cells, algal cells, insect cells, and plant cells. In some embodiments, yeasts useful in the present methods include yeasts that have been deposited with microorganism depositories (e.g. IFO, ATCC, etc.) and belong to the genera Aciculoconidium, Ambrosiozyma, Arthroascus, Arxiozyma, Ashbya, Babjevia, Bensingtonia, Botryoascus, Botryozyma, Brettanomyces, Bullera, Bulleromyces, Candida, Citeromyces, Clavispora, Cryptococcus, Cystofilobasidium, Debaryomyces, Dekkara, Dipodascopsis, Dipodascus, Eeniella, Endomycopsella, Eremascus, Eremothecium, Erythrobasidium, Fellomyces, Filobasidium, Galactomyces, Geotrichum, Guilliermondella, Hanseniaspora, Hansenula, Hasegawaea, Holtermannia, Hormoascus, Hyphopichia, Issatchenkia, Kloeckera, Kloeckeraspora, Kluyveromyces, Kondoa, Kuraishia, Kurtzmanomyces, Leucosporidium, Lipomyces, Lodderomyces, Malassezia, Metschnikowia, Mrakia, Myxozyma, Nadsonia, Nakazawaea, Nematospora, Ogataea, Oosporidium, Pachysolen, Phachytichospora, Phaffia, Pichia, Rhodosporidium, Rhodotorula, Saccharomyces, Saccharomycodes, Saccharomycopsis, Saitoella, Sakaguchia, Saturnospora, Schizoblastosporion, Schizosaccharomyces, Schwanniomyces, Sporidiobolus, Sporobolomyces, Sporopachydermia, Stephanoascus, Sterigmatomyces, Sterigmatosporidium, Symbiotaphrina, Sympodiomyces, Sympodiomycopsis, Torulaspora, Trichosporiella, Trichosporon, Trigonopsis, Tsuchiyaea, Udeniomyces, Waltomyces, Wickerhamia, Wickerhamiella, Williopsis, Yamadazyma, Yarrowia, Zygoascus, Zygosaccharomyces, Zygowilliopsis, and Zygozyma, among others.

In some embodiments, the host cell is Saccharomyces cerevisiae, Pichia pastoris, Schizosaccharomyces pombe, Dekkera bruxellensis, Kluyveromyces lactis (previously called Saccharomyces lactis), Kluveromyces marxianus, Arxula adeninivorans, or Hansenula polymorpha (now known as Pichia angusta). In some embodiments, the host cell is a strain of the genus Candida, such as Candida lipolytica, Candida guilliermondii, Candida krusei. Candida pseudotropicalis, or Candida utilis.

In a particular embodiment, the host cell is Saccharomyces cerevisiae. In some embodiments, the host is a strain of Saccharomyces cerevisiae selected from the group consisting of Baker's yeast, CBS 7959, CBS 7960, CBS 7961, CBS 7962, CBS 7963, CBS 7964, IZ-1904. TA, BG-1, CR-1, SA-1, M-26, Y-904, PE-2, PE-5, VR-1, BR-1, BR-2, ME-2. VR-2, MA-3, MA-4, CAT-1, CB-1, NR-1, BT-1, CEN.PK, CEN.PK2, and AL-1. In some embodiments, the host cell is a strain of Saccharomyces cerevisiae selected from the group consisting of PE-2, CAT-1, VR-1, BG-1, CR-1, and SA-1. In a particular embodiment, the strain of Saccharomyces cerevisiae is PE-2. In another particular embodiment, the strain of Saccharomyces cerevisiae is CAT-1. In another particular embodiment, the strain of Saccharomyces cerevisiae is BG-1.

In some embodiments, the host cell is a microbe that is suitable for industrial fermentation. In particular embodiments, the microbe is conditioned to subsist under high solvent concentration, high temperature, expanded substrate utilization, nutrient limitation, osmotic stress due to sugar and salts, acidity, sulfite, and bacterial contamination, or combinations thereof, which are recognized stress conditions of the industrial fermentation environment.

### 7.2.3 Mevalonate Pathway

In some embodiments, the cell provided herein comprises one or more enzymes of the mevalonate (MEV) pathway. In some embodiments, the one or more enzymes of the MEV pathway comprise an enzyme that condenses acetyl-CoA with malonyl-CoA to form acetoacetyl-CoA. In some embodiments, the one or more enzymes of the MEV pathway comprise an enzyme that condenses two molecules of acetyl-CoA to form acetoacetyl-CoA. In some embodiments, the one or more enzymes of the MEV pathway comprise an enzyme that condenses acetoacetyl-CoA with acetyl-CoA to form HMG-CoA. In some embodiments, the one or more enzymes of the MEV pathway comprise an enzyme that converts HMG-CoA to mevalonate. In some embodiments, the one or more enzymes of the MEV pathway comprise an enzyme that phosphorylates mevalonate to mevalonate 5-phosphate. In some embodiments, the one or more enzymes of the MEV pathway comprise an enzyme that converts mevalonate 5-phosphate to mevalonate 5-pyrophosphate. In some embodiments, the one or more enzymes of the MEV pathway comprise an enzyme that converts mevalonate 5-pyrophosphate to isopentenyl pyrophosphate.

In some embodiments, the one or more enzymes of the MEV pathway are selected from the group consisting of acetyl-CoA thiolase, acetoacetyl-CoA synthetase, HMG-COA synthase, HMG-COA reductase, mevalonate kinase, phosphomevalonate kinase and mevalonate pyrophosphate decarboxylase. In some embodiments, with regard to the enzyme of the MEV pathway capable of catalyzing the formation of acetoacetyl-CoA, the genetically modified host cell comprises either an enzyme that condenses two molecules of acetyl-CoA to form acetoacetyl-CoA, e.g., acetyl-CoA thiolase; or an enzyme that condenses acetyl-CoA with malonyl-CoA to form acetoacetyl-CoA, e.g., acetoacetyl-CoA synthase. In some embodiments, the genetically modified host cell comprises both an enzyme that condenses two molecules of acetyl-CoA to form acetoacetyl-CoA, e.g., acetyl-CoA thiolase; and an enzyme that condenses acetyl-CoA with malonyl-CoA to form acetoacetyl-CoA, e.g., acetoacetyl-CoA synthase.

In some embodiments, the host cell comprises more than one enzyme of the MEV pathway. In some embodiments, the host cell comprises two enzymes of the MEV pathway. In some embodiments, the host cell comprises an enzyme that can convert HMG-CoA into mevalonate and an enzyme that can convert mevalonate into mevalonate 5-phosphate. In some embodiments, the host cell comprises three enzymes of the MEV pathway. In some embodiments, the host cell comprises four enzymes of the MEV pathway. In some embodiments, the host cell comprises five enzymes of the MEV pathway. In some embodiments, the host cell comprises six enzymes of the MEV pathway. In some embodiments, the host cell seven enzymes of the MEV pathway. In some embodiments, the host cell comprises all of the enzymes of the MEV pathway.

In some embodiments, the cell further comprises an enzyme that can convert isopentenyl pyrophosphate (IPP) into dimethylallyl pyrophosphate (DMAPP). In some embodiments, the cell further comprises an enzyme that can condense IPP and/or DMAPP molecules to form a polyprenyl compound. In some embodiments, the cell further comprises an enzyme that can modify IPP or a polyprenyl to form an isoprenoid compound.

### 7.2.3.1 Conversion of Acetyl-CoA to Acetoacetyl-CoA

In some embodiments, the genetically modified host cell comprises an enzyme that can condense two molecules of acetyl-coenzyme A to form acetoacetyl-CoA, e.g., an acetyl-CoA thiolase. Illustrative examples of nucleotide sequences encoding such an enzyme include, but are not limited to: (NC_000913 REGION: 2324131.2325315; Escherichia coli), (D49362; Paracoccus denitrificans), and (L20428; Saccharomyces cerevisiae). Acetyl-CoA thiolase catalyzes the reversible condensation of two molecules of acetyl-CoA to yield acetoacetyl-CoA, but this reaction is thermodynamically unfavorable; acetoacetyl-CoA thiolysis is favored over acetoacetyl-CoA synthesis. Acetoacetyl-CoA synthase (AACS) (alternately referred to as acetyl-CoA:malonyl-CoA acyltransferase; EC 2.3.1.194) condenses acetyl-CoA with malonyl-CoA to form acetoacetyl-CoA. In contrast to acetyl-CoA thiolase, AACS-catalyzed acetoacetyl-CoA synthesis is essentially an energy-favored reaction, due to the associated decarboxylation of malonyl-CoA. In addition, AACS exhibits no thiolysis activity against acetoacetyl-CoA, and thus the reaction is irreversible. Thus, in other embodiments, the genetically modified host cell provided herein utilizes an acetoacetyl-CoA synthase to form acetoacetyl-CoA from acetyl-CoA and malonyl-CoA.

In some embodiments, the AACS is from Streptomyces sp. strain CL190 (Okamura et al., Proc Natl Acad Sci USA 107 (25): 11265-70 (2010). Representative AACS nucleotide sequences of Streptomyces sp. strain CL190 include accession number AB540131.1, and SEQ ID NO:19 of U.S. Pat. Pub. No. 2014/0273144. Representative AACS protein sequences of Streptomyces sp. strain CL190 include accession numbers D7URV0, BAJ10048, and SEQ ID NO:20 of U.S. Pat. Pub. No. 2014/0273144. Other acetoacetyl-CoA synthases useful for the compositions and methods provided herein include, but are not limited to, Streptomyces sp. (AB183750; KO-3988 BAD86806); S. anulatus strain 9663 (FN178498; CAX48662); Streptomyces sp. KO-3988 (AB212624; BAE78983); Actinoplanes sp. A40644 (AB113568; BAD07381); Streptomyces sp. C (NZ_ACEW010000640; ZP_05511702); Nocardiopsis dassonvillei DSM 43111 (NZ_ABUI01000023; ZP_04335288); Mycobacterium ulcerans Agy99 (NC_008611; YP_907152); Mycobacterium marinum M (NC_010612; YP_001851502); Streptomyces sp. Mg1 (NZ_DS570501; ZP_05002626); Streptomyces sp. AA4 (NZ_ACEV01000037; ZP_05478992); S. roseosporus NRRL 15998 (NZ_ABYB01000295; ZP_04696763); Streptomyces sp. ACTE (NZ_ADFD01000030; ZP_06275834); S. viridochromogenes DSM 40736 (NZ_ACEZ01000031; ZP_05529691); Frankia sp. CcI3 (NC_007777; YP_480101); Nocardia brasiliensis (NC_018681; YP_006812440.1); and Austwickia chelonae (NZ_BAGZ01000005; ZP_10950493.1). Additional suitable acetoacetyl-CoA synthases include those described in U.S. Patent Application Publication Nos. 2010/0285549 and 2011/0281315, the contents of which are incorporated by reference in their entireties.

Acetoacetyl-CoA synthases also useful in the compositions and methods provided herein include those molecules which are said to be “derivatives” of any of the acetoacetyl-CoA synthases described herein. Such a “derivative” has the following characteristics: (1) it shares substantial homology with any of the acetoacetyl-CoA synthases described herein; and (2) is capable of catalyzing the irreversible condensation of acetyl-CoA with malonyl-CoA to form acetoacetyl-CoA. A derivative of an acetoacetyl-CoA synthase is said to share “substantial homology” with acetoacetyl-CoA synthase if the amino acid sequences of the derivative is at least 80%, and more preferably at least 90%, and most preferably at least 95%, the same as that of acetoacetyl-CoA synthase.

### 7.2.3.2 Conversion of Acetoacetyl-CoA to HMG-COA

In some embodiments, the host cell comprises an enzyme that can condense acetoacetyl-CoA with another molecule of acetyl-CoA to form 3-hydroxy-3-methylglutaryl-CoA (HMG-COA), e.g., a HMG-COA synthase. Illustrative examples of nucleotide sequences encoding such an enzyme include, but are not limited to: (NC_001145. complement 19061.20536; Saccharomyces cerevisiae), (X96617; Saccharomyces cerevisiae), (X83882; Arabidopsis thaliana), (AB037907; Kitasatospora griseola), (BT007302; Homo sapiens), and (NC_002758, Locus tag SAV2546, GeneID 1122571; Staphylococcus aureus).

### 7.2.3.3 Conversion of HMG-COA to Mevalonate

In some embodiments, the host cell comprises an enzyme that can convert HMG-COA into mevalonate, e.g., a HMG-COA reductase. In some embodiments, HMG-COA reductase is an NADPH-using hydroxymethylglutaryl-CoA reductase-CoA reductase. Illustrative examples of nucleotide sequences encoding an NADPH-using HMG-COA reductase include, but are not limited to: (NM_206548; Drosophila melanogaster), (NC_002758, Locus tag SAV2545, GeneID 1122570; Staphylococcus aureus). (AB015627: Streptomyces sp. KO 3988). (AX128213. providing the sequence encoding a truncated HMG-COA reductase; Saccharomyces cerevisiae), and (NC_001145: complement (115734.118898; Saccharomyces cerevisiae).

In some embodiments, HMG-COA reductase is an NADH-using hydroxymethylglutaryl-CoA reductase-CoA reductase. HMG-COA reductases (EC 1.1.1.34; EC 1.1.1.88) catalyze the reductive deacylation of(S)-HMG-COA to (R)-mevalonate, and can be categorized into two classes, class I and class II HMGrs. Class I includes the enzymes from eukaryotes and most archaea, and class II includes the HMG-COA reductases of certain prokaryotes and archaea. In addition to the divergence in the sequences, the enzymes of the two classes also differ with regard to their cofactor specificity. Unlike the class I enzymes, which utilize NADPH exclusively, the class II HMG-COA reductases vary in the ability to discriminate between NADPH and NADH. See, e.g., Hedl et al., Journal of Bacteriology 186 (7): 1927-1932 (2004). Co-factor specificities for select class II HMG-COA reductases are provided below.

TABLE 2
Co-factor specificities for select class II HMG-CoA reductases

| Source | Coenzyme specificity | Kₘ^{NADPH} (μM) | Kₘ^{NADH} (μM) |
|---|---|---|---|
| P. mevalonii | NADH | | 80 |
| A. fulgidus | NAD(P)H | 500 | 160 |
| S. aureus | NAD(P)H | 70 | 100 |
| E. faecalis | NADPH | 30 | |

Useful HMG-COA reductases for the compositions and methods provided herein include HMG-COA reductases that are capable of utilizing NADH as a cofactor, e.g., HMG-CoA reductase from P. mevalonii, A. fulgidus or S. aureus. In particular embodiments, the HMG-COA reductase is capable of only utilizing NADH as a cofactor, e.g., HMG-COA reductase from P. mevalonii, S. pomeroyi or D. acidovorans.

In some embodiments, the NADH-using HMG-COA reductase is from Pseudomonas mevalonii. The sequence of the wild-type mvaA gene of Pseudomonas mevalonii, which encodes HMG-CoA reductase (EC 1.1.1.88), has been previously described. See Beach and Rodwell. J. Bacteriol. 171:2994-3001 (1989). Representative mvaA nucleotide sequences of Pseudomonas mevalonii include accession number M24015, and SEQ ID NO: 21 of U.S. Pat. Pub. No. 2014/0273144. Representative HMG-COA reductase protein sequences of Pseudomonas mevalonii include accession numbers AAA25837, P13702, MVAA_PSEMV, and SEQ ID NO: 22 of U.S. Pat. Pub. No. 2014/0273144.

In some embodiments, the NADH-using HMG-COA reductase is from Silicibacter pomeroyi. Representative HMG-COA reductase nucleotide sequences of Silicibacter pomeroyi include accession number NC_006569.1, and SEQ ID NO: 23 of U.S. Pat. Pub. No. 2014/0273144. Representative HMG-COA reductase protein sequences of Silicibacter pomeroyi include accession number YP_164994, and SEQ ID NO: 24 of U.S. Pat. Pub. No. 2014/0273144.

In some embodiments, the NADH-using HMG-COA reductase is from Delftia acidovorans. A representative HMG-COA reductase nucleotide sequences of Delftia acidovorans includes NC_010002 REGION: complement (319980.321269), and SEQ ID NO: 25 of U.S. Pat. Pub. No. 2014/0273144. Representative HMG-COA reductase protein sequences of Delftia acidovorans include accession number YP_001561318, and SEQ ID NO: 26 of U.S. Pat. Pub. No. 2014/0273144.

In some embodiments, the NADH-using HMG-COA reductases is from Solanum tuberosum (Crane et al., J. Plant Physiol. 159:1301-1307 (2002)).

NADH-using HMG-COA reductases also useful in the compositions and methods provided herein include those molecules which are said to be “derivatives” of any of the NADH-using HMG-COA reductases described herein, e.g., from P. mevalonii, S. pomeroyi and D. acidovorans. Such a “derivative” has the following characteristics: (1) it shares substantial homology with any of the NADH-using HMG-CoA reductases described herein; and (2) is capable of catalyzing the reductive deacylation of(S)-HMG-CoA to (R)-mevalonate while preferentially using NADH as a cofactor. A derivative of an NADH-using HMG-COA reductase is said to share “substantial homology” with NADH-using HMG-COA reductase if the amino acid sequences of the derivative is at least 80%, and more preferably at least 90%, and most preferably at least 95%, the same as that of NADH-using HMG-COA reductase.

As used herein, the phrase “NADH-using” means that the NADH-using HMG-COA reductase is selective for NADH over NADPH as a cofactor, for example, by demonstrating a higher specific activity for NADH than for NADPH. In some embodiments, selectivity for NADH as a cofactor is expressed as a k_{cat}^{(NADH)}/K_{cat}^{(NADPH)} ratio. In some embodiments, the NADH-using HMG-COA reductase has a k_{cat}^{(NADH)}/k_{cat}^{(NADPH)} ratio of at least 5, 10, 15, 20, 25 or greater than 25. In some embodiments, the NADH-using HMG-CoA reductase uses NADH exclusively. For example, an NADH-using HMG-COA reductase that uses NADH exclusively displays some activity with NADH supplied as the sole cofactor in vitro, and displays no detectable activity when NADPH is supplied as the sole cofactor. Any method for determining cofactor specificity known in the art can be utilized to identify HMG-COA reductases having a preference for NADH as cofactor, including those described by Kim et al., Protein Science 9:1226-1234 (2000); and Wilding et al., J. Bacteriol. 182 (18): 5147-52 (2000), the contents of which are hereby incorporated in their entireties.

In some embodiments, the NADH-using HMG-COA reductase is engineered to be selective for NADH over NAPDH, for example, through site-directed mutagenesis of the cofactor-binding pocket. Methods for engineering NADH-selectivity are described in Watanabe et al., Microbiology 153:3044-3054 (2007), and methods for determining the cofactor specificity of HMG-CoA reductases are described in Kim et al., Protein Sci. 9:1226-1234 (2000), the contents of which are hereby incorporated by reference in their entireties.

In some embodiments, the NADH-using HMG-COA reductase is derived from a host species that natively comprises a mevalonate degradative pathway, for example, a host species that catabolizes mevalonate as its sole carbon source. Within these embodiments, the NADH-using HMG-CoA reductase, which normally catalyzes the oxidative acylation of internalized (R)-mevalonate to(S)-HMG-COA within its native host cell, is utilized to catalyze the reverse reaction, that is, the reductive deacylation of(S)-HMG-COA to (R)-mevalonate, in a genetically modified host cell comprising a mevalonate biosynthetic pathway. Prokaryotes capable of growth on mevalonate as their sole carbon source have been described by: Anderson et al., J. Bacteriol, 171 (12): 6468-6472 (1989); Beach et al., J. Bacteriol. 171:2994-3001 (1989); Bensch et al., J. Biol. Chem. 245:3755-3762; Fimongnari et al., Biochemistry 4:2086-2090 (1965); Siddiqi et al., Biochem. Biophys. Res. Commun. 8:110-113 (1962); Siddiqi et al., J. Bacteriol. 93:207-214 (1967); and Takatsuji et al., Biochem. Biophys. Res. Commun. 110:187-193 (1983), the contents of which are hereby incorporated by reference in their entireties.

In some embodiments of the compositions and methods provided herein, the host cell comprises both a NADH-using HMGr and an NADPH-using HMG-COA reductase.

### 7.2.3.4 Conversion of Mevalonate to Mevalonate-5-Phosphate

In some embodiments, the host cell comprises an enzyme that can convert mevalonate into mevalonate 5-phosphate, e.g., a mevalonate kinase. Illustrative examples of nucleotide sequences encoding such an enzyme include, but are not limited to: (L77688; Arabidopsis thaliana), and (X55875; Saccharomyces cerevisiae).

### 7.2.3.5 Conversion of Mevalonate-5-Phosphate to Mevalonate-5-Pyrophosphate

In some embodiments, the host cell comprises an enzyme that can convert mevalonate 5-phosphate into mevalonate 5-pyrophosphate, e.g., a phosphomevalonate kinase. Illustrative examples of nucleotide sequences encoding such an enzyme include, but are not limited to: (AF429385; Hevea brasiliensis), (NM_006556; Homo sapiens), and (NC_001145. complement 712315.713670; Saccharomyces cerevisiae).

### 7.2.3.6 Conversion of Mevalonate-5-Pyrophosphate to IPP

In some embodiments, the host cell comprises an enzyme that can convert mevalonate 5-pyrophosphate into isopentenyl diphosphate (IPP), e.g., a mevalonate pyrophosphate decarboxylase. Illustrative examples of nucleotide sequences encoding such an enzyme include, but are not limited to: (X97557; Saccharomyces cerevisiae). (AF290095; Enterococcus faecium), and (U49260; Homo sapiens).

### 7.2.3.7 Conversion of IPP to DMAPP

In some embodiments, the host cell further comprises an enzyme that can convert IPP generated via the MEV pathway into dimethylallyl pyrophosphate (DMAPP), e.g., an IPP isomerase. Illustrative examples of nucleotide sequences encoding such an enzyme include, but are not limited to: (NC_000913, 3031087.3031635; Escherichia coli), and (AF082326; Haematococcus pluvialis).

### 7.2.4 Polyprenyl Synthases

In some embodiments, the host cell comprises a polyprenyl synthase that can condense IPP and/or DMAPP molecules to form polyprenyl compounds containing more than five carbons.

In some embodiments, the host cell comprises an enzyme that can combine IPP and DMAPP or IPP and FPP to form geranylgeranyl pyrophosphate (“GGPP”), also referred to as GGPP synthase (EC 2.5.1.29). Illustrative examples of nucleotide sequences that encode such an enzyme include, but are not limited to: (ATHGERPYRS; Arabidopsis thaliana), (BT005328; Arabidopsis thaliana), (NM_119845; Arabidopsis thaliana), (NZ_AAJM01000380, Locus ZP_00743052; Bacillus thuringiensis serovar israelensis, ATCC 35646 sq1563), (CRGGPPS; Catharanthus roseus), (NZ_AABF02000074, Locus ZP_00144509; Fusobacterium nucleatum subsp. vincentii, ATCC 49256), (GFGGPPSGN; Gibberella fujikuroi), (AY371321; Ginkgo biloba), (AB055496; Hevea brasiliensis), (AB017971; Homo sapiens), (MCI276129; Mucor circinelloides f. lusitanicus), (AB016044; Mus musculus), (AABX01000298, Locus NCU01427; Neurospora crassa), (NCU20940; Neurospora crassa), (NZ_AAKL01000008, Locus ZP_00943566; Ralstonia solanacearum UW551), (AB118238; Rattus norvegicus), (SCU31632; Saccharomyces cerevisiae), (AB016095; Synechococcus elongates), (SAGGPS; Sinapis alba), (SSOGDS; Sulfolobus acidocaldarius), (NC_007759, Locus YP_461832; Syntrophus aciditrophicus SB), (NC_006840, Locus YP_204095: Vibrio fischeri ES114), (NM_112315; Arabidopsis thaliana), (ERWCRTE; Pantoea agglomerans), (D90087, Locus BAA14124; Pantoea ananatis), (X52291, Locus CAA36538; Rhodobacter capsulatus), (AF195122, Locus AAF24294; Rhodobacter sphaeroides), (NC_004350, Locus NP_721015; Streptococcus mutans UA159), (NP_015256; Saccharomyces cerevisiae); (AFC92798; Blakeslea trispora), (BAA14124; Pantoea ananatis), (AAM21639; Cistus creticus); (AAY33921; Xanthophyllomyces dendrorhous), (XP_019067954; Solanum lycopersicum).

### 7.2.5 Carotenoid Pathway

In some embodiments, the host cell comprises a heterologous nucleic acid encoding a phytoene synthase which can catalyze the conversion of geranylgeranyl pyrophosphate to phytoene. Illustrative examples of suitable polypeptide sequences or polynucleotides that encode a phytoene synthase include, but are not limited to: CrtB (Lamprocystis purpurea; GenBank Protein Acc.: WP_020503292) (EC 2.5.1.99 or EC 2.5.1.32).

In some embodiments, the host cell comprises a heterologous nucleic acid encoding a lycopene cyclase which can catalyse the conversion of lycopene to beta-carotene and/or the conversion of neurosporene to 7,8-dihydro-beta-carotene. Illustrative examples of suitable polypeptide sequences or polynucleotides that encode a lycopene cyclase include, but are not limited to: CrtY (Pantoea ananatis; GenBank Protein Acc.: BAA14126) (EC 5.5.1.19).

In some embodiments, the host cell comprises a heterologous nucleic acid encoding a bifunctional enzyme which can catalyse the conversion of geranylgeranyl pyrophosphate to phytoene (phytoene synthase) and the conversion of lycopene to β-carotene (lycopene cyclase) and/or the conversion of neurosporene to 7,8-dihydro-beta-carotene. Illustrative examples of suitable polypeptide sequences or polynucleotides that encode such bifunctional enzyme include: CrtY/B (Xanthophyllomyces dendrorhous; Verwaal et al. App. Environ. 733 (13): 4342-50 (2007); CrtY/B (Phycomyces blakesleeanus; GenBank Protein Acc.: XP_018294563), CrtY/B (Neurospora crassa, GenBank Protein Acc.: XP_965725), and CrtY/B (Blakeslea trispora; GenBank Protein Acc.: AAO46893).

In some embodiments, the host cell comprises a heterologous nucleic acid encoding a phytoene desaturase which can catalyse the conversion of phytoene to neurosporene and/or the conversion of neurosporene to lycopene. Illustrated examples of suitable polypeptide sequences or polynucleotides that encode a phytoene desaturase include, but are not limited to: CrtI (Xanthophyllomyces dendrorhous; Verwaal et al. App. Environ. Microbiol. 73 (130:4342-50, 2007) CrtI (Xanthophyllomyces dendrorhous; GenBank Protein Acc.: CAA75240), CrtI (Mycobacterium goodie; GenBank Protein Acc.: WP_049747535), CrtI (Neurospora crassa; GenBank Protein Acc.: XP_964713), CrtI (Paenibacillus_sp; GenBank Protein Acc.: WP_042140268), and CrtI (Bradyrhizobium_sp; GenBank Protein Acc.: WP_011924720) (EC 1.3.99.31 or EC 1.3.5.5 or EC 1.3.5.6 or EC 1.3.99.28 or EC 1.3.99.30).

In some embodiments, the host cell comprises a heterologous nucleic acid encoding beta-carotene ketolase which can catalyze the conversion of beta-carotene to echinenone, the conversion of echinenone to canthaxanthin, the conversion of beta-cryptoxanthin to 3-hydroxyechinenone/3′-hydroxyechinenone, the conversion of 3-hycroxyechinenone/3′-hydroxyechinenone to phenicoxanthin, the conversion of zeaxanthin to adonixanthin, and/or the conversion of adonixanthin to astaxanthin. Illustrated examples of suitable polypeptide sequences or polynucleotides include, but are not limited to: CrtW (Paracoccus_sp.; GenBank Protein Acc.: BAA09591), CrtW (Brevundimonas sp; GenBank Protein Acc.: BAD99406), CrtW (Haematococcus lacustris; GenBank Protein Acc.: ADN43075), CrtW (Chlamydomonas reinhardtii; XP_001698699), CrtW (Sphingomonas sp. 1PNM-20; GenBank Protein Acc.: WP_095996876); CrtW (Paracoccus sp. Strain N81106 (Agrobacterium aurantiacum) GenBank ID: BAE47465.1; UniProt CRTW_PARSN; Ukibe et al., Appl. Environ. Microbiol. 75 (22): 7205-7211 (2009)); HpBkt (Haematococcus pluvialis; GenBank ID D45881.1; BAA08300.1) (EC 1.14.11.B16 or EC 1.3.5.B4).

### 7.2.6 Modifications to Increase Acetyl-CoA Levels

In certain embodiments, any of the above cells producing compounds from acetyl-CoA, comprise modifications to their acetyl-CoA pathways according to U.S. 2014/0273144 A1. In certain embodiments, the cells comprise a phosphoketolase (PK; EC 4.1.2.9) and a functional disruption of an endogenous enzyme that converts acetyl phosphate to acetate. In certain embodiments, the cells comprise a phosphotransacetylase (PTA; EC 2.3.1.8); and a functional disruption of an endogenous enzyme that converts acetyl phosphate to acetate. In some embodiments, the enzyme that converts acetyl phosphate to acetate is a glycerol-1-phosphatase (EC 3.1.3.21). In some embodiments, the glycerol-1-phosphatase is selected from the group consisting of GPP1/RHR2, GPP2HOR2, and homologues and variants thereof. In some embodiments, the host cell comprises a functional disruption of GPP1/RHR2. In some embodiments, the host cell comprises a functional disruption of GPP2/HOR2. In some embodiments, the host cell comprises a functional disruption of both GPP1/RHR2 and GPP2/HOR2. In some embodiments, the host cell further comprises an acylating acetylaldehyde dehydrogenase (ADA; EC 1.2.1.10). In some embodiments, host cell further comprises a functional disruption of one or more enzymes of the native pyruvate dehydrogenase (PDH)-bypass. In some embodiments, the one or more enzymes of the PDH-bypass are selected from acetyl-CoA synthetase 1 (ACS1), acetyl-CoA synthetase 2 (ACS2), and aldehyde dehydrogenase 6 (ALD6).

### 7.3 Methods of Producing Canthaxanthin

In certain embodiments, provided herein is a method for the production of canthaxanthin. The method comprises: (a) culturing, in a culture medium with a carbon source, a genetically modified host cell described herein; and (b) recovering the canthaxanthin. In certain embodiments, the fermentation is performed by culturing the genetically modified host cells in a culture medium comprising a carbon source under suitable culture conditions for a period of time sufficient to produce a desired biomass of the host cells and/or a desired amount of canthaxanthin.

In certain embodiments, the fermentation process is carried out in two stages—a build stage and a production stage. The build stage is carried out for a period of time sufficient to produce an amount of cellular biomass that can support production of canthaxanthin during the production stage. The build stage is carried out for a period of time sufficient for the population present at the time of inoculation to undergo a plurality of doublings until a desired cell density is reached. In some embodiments, the build stage is carried out for a period of time sufficient for the host cell population to reach a cell density (OD₆₀₀) of between 0.01 and 400 in the fermentation vessel or container in which the build stage is being carried out. In some embodiments, the build stage is carried out until an OD₆₀₀ of at least 0.01 is reached. In some embodiments, the build stage is carried out until an OD₆₀₀ of at least 0.1 is reached. In some embodiments, the build stage is carried out until an OD₆₀₀ of at least 1.0 is reached. In some embodiments, the build stage is carried out until an OD₆₀₀ of at least 10 is reached. In some embodiments, the build stage is carried out until an OD₆₀₀ of at least 100 is reached. In some embodiments, the build stage is carried out until an OD₆₀₀ of between 0.01 and 100 is reached. In some embodiments, the build stage is carried out until an OD₆₀₀ of between 0.1 and 10 is reached. In some embodiments, the build stage is carried out until an OD₆₀₀ of between 1 and 100 is reached. In other embodiments, the build stage is carried for a period of at least 12, 24, 36, 48, 60, 72, 84, 96 or more than 96 hours.

In some embodiments, the production stage is carried out for a period of time sufficient to produce a desired amount of canthaxanthin. In some embodiments, the production stage is carried out for a period of at least 12, 24, 36, 48, 60, 72, 84, 96 or more than 96 hours. In some embodiments, the production stage is carried out for a period of between 3 and 20 days. In some embodiments, the production stage is carried for a period of 1, 2, 3, 4, 5, 6, 7, 8, 9, 10, 11, 12, 13, 14, 15, 16, 17, 18, 19, 20 or more than 20 days.

In a particular embodiment, the method of producing canthaxanthin comprises conducting fermentation of the genetically modified host cell under aerobic conditions sufficient to allow growth and maintenance of the genetically modified host cell; then subsequently providing microaerobic fermentation conditions sufficient to induce production of canthaxanthin, and maintaining the microaerobic conditions throughout the fermentation run. In certain embodiments, the microaerobic conditions are used throughout the fermentation run. In certain embodiments, the aerobic conditions arc used throughout the fermentation run.

In certain embodiments, the production of the elevated level of canthaxanthin by the host cell is inducible by an inducing compound. Such a host cell can be manipulated with ease in the absence of the inducing compound. The inducing compound is then added to induce the production of the elevated level of canthaxanthin by the host cell. In other embodiments, production of the elevated level of canthaxanthin by the host cell is inducible by changing culture conditions, such as, for example, the growth temperature, media constituents, and the like.

In certain embodiments, an inducing agent is added during the production stage to activate a promoter or to relieve repression of a transcriptional regulator associated with a biosynthetic pathway to promote production of canthaxanthin. In certain embodiments, an inducing agent is added during the build stage to repress a promoter or to activate a transcriptional regulator associated with a biosynthetic pathway to repress the production of canthaxanthin, and an inducing agent is removed during the production stage to activate a promoter to relieve repression of a transcriptional regulator to promote the production of canthaxanthin. The term “genetic switch” is used herein to refer to the use of a promoter or other genetic elements to control activation or de-activation of the biosynthetic pathway for the isoprenoid production. Illustrative examples of useful inducing agent or a genetic switch for controlling target isoprenoid production are described in, e.g., PCT Application Publications WO2015/020649, WO2016/210343, and WO2016210350, which are incorporated herein by reference in their entirety.

In another embodiment, the method of producing canthaxanthin comprises culturing host cells in separate build and production culture media. For example, the method can comprise culturing the genetically modified host cell in a build stage wherein the cell is cultured under non-producing conditions (e.g., non-inducing conditions) to produce an inoculum, then transferring the inoculum into a second fermentation medium under conditions suitable to induce canthaxanthin production (e.g., inducing conditions), and maintaining steady state conditions in the second fermentation stage to produce a cell culture containing canthaxanthin.

In certain embodiments, the biosynthetic pathway for canthaxanthin can be under the control of the same genetic switch or an inducer. For example, the biosynthetic pathways for the production of canthaxanthin may be under the control of pGal promoters, which are regulated by the Gal regulon. Examples of the Gal regulon which are further repressed or induced by a maltose are described in PCT Application Publications WO2015/020649, WO2016/210343, and WO2016210350.

In some embodiments, the genetically modified host cell produces an increased amount of canthaxanthin to a parent cell not comprising the one or more modifications, or a parent cell comprising only a subset of the one or more modifications of the genetically modified host cell, but is otherwise genetically identical. In some embodiments, the increased amount is at least 1%, 5%, 10%, 15%, 20%, 25%, 30%, 35%, 40%, 45%, 50%, 55%, 60%, 65%, 70%, 75%, 80%, 85%, 90%, 95%, 100% or greater than 100%, as measured, for example, in yield, production, productivity, in grams per liter of cell culture, milligrams per gram of dry cell weight, on a per unit volume of cell culture basis, on a per unit dry cell weight basis, on a per unit volume of cell culture per unit time basis, or on a per unit dry cell weight per unit time basis.

In some embodiments, the host cell produces an elevated level of canthaxanthin that is greater than about 10 grams per liter of fermentation medium. In some such embodiments, the canthaxanthin is produced in an amount from about 10 to about 50 grams, more than about 15 grams, more than about 20 grams, more than about 25 grams, or more than about 30 grams per liter of cell culture.

In some embodiments, the host cell produces an elevated level of canthaxanthin that is greater than about 50 milligrams per gram of dry cell weight. In some such embodiments, the canthaxanthin is produced in an amount from about 50 to about 1500 milligrams, more than about 100 milligrams, more than about 150 milligrams, more than about 200 milligrams, more than about 250 milligrams, more than about 500 milligrams, more than about 750 milligrams, or more than about 1000 milligrams per gram of dry cell weight.

In some embodiments, the host cell produces an elevated level of canthaxanthin that is at least about 10%, at least about 15%, at least about 20%, at least about 25%, at least about 30%, at least about 35%, at least about 40%, at least about 45%, at least about 50%, at least about 60%, at least about 70%, at least about 80%, at least about 90%, at least about 2-fold, at least about 2.5-fold, at least about 5-fold, at least about 10-fold, at least about 20-fold, at least about 30-fold, at least about 40-fold, at least about 50-fold, at least about 75-fold, at least about 100-fold, at least about 200-fold, at least about 300-fold, at least about 400-fold, at least about 500-fold, or at least about 1,000-fold, or more, higher than the level of canthaxanthin produced by a parent cell, on a per unit volume of cell culture basis.

In some embodiments, the host cell produces an elevated level of canthaxanthin that is at least about 10%, at least about 15%, at least about 20%, at least about 25%, at least about 30%, at least about 35%, at least about 40%, at least about 45%, at least about 50%, at least about 60%, at least about 70%, at least about 80%, at least about 90%, at least about 2-fold, at least about 2.5-fold, at least about 5-fold, at least about 10-fold, at least about 20-fold, at least about 30-fold, at least about 40-fold, at least about 50-fold, at least about 75-fold, at least about 100-fold, at least about 200-fold, at least about 300-fold, at least about 400-fold, at least about 500-fold, or at least about 1,000-fold, or more, higher than the level of canthaxanthin produced by the parent cell, on a per unit dry cell weight basis.

In some embodiments, the host cell produces an elevated level of canthaxanthin that is at least about 10%, at least about 15%, at least about 20%, at least about 25%, at least about 30%, at least about 35%, at least about 40%, at least about 45%, at least about 50%, at least about 60%, at least about 70%, at least about 80%, at least about 90%, at least about 2-fold, at least about 2.5-fold, at least about 5-fold, at least about 10-fold, at least about 20-fold, at least about 30-fold, at least about 40-fold, at least about 50-fold, at least about 75-fold, at least about 100-fold, at least about 200-fold, at least about 300-fold, at least about 400-fold, at least about 500-fold, or at least about 1,000-fold, or more, higher than the level of canthaxanthin produced by the parent cell, on a per unit volume of cell culture per unit time basis.

In some embodiments, the host cell produces an elevated level of canthaxanthin that is at least about 10%, at least about 15%, at least about 20%, at least about 25%, at least about 30%, at least about 35%, at least about 40%, at least about 45%, at least about 50%, at least about 60%, at least about 70%, at least about 80%, at least about 90%, at least about 2-fold, at least about 2.5-fold, at least about 5-fold, at least about 10-fold, at least about 20-fold, at least about 30-fold, at least about 40-fold, at least about 50-fold, at least about 75-fold, at least about 100-fold, at least about 200-fold, at least about 300-fold, at least about 400-fold, at least about 500-fold, or at least about 1,000-fold, or more, higher than the level of canthaxanthin produced by the parent cell, on a per unit dry cell weight per unit time basis.

### 7.4 Culture Media and Conditions

Materials and methods for the maintenance and growth of microbial cultures are well known to those skilled in the art of microbiology or fermentation science (see, for example, Bailey et al., Biochemical Engineering Fundamentals, second edition, McGraw Hill, New York, 1986). Consideration must be given to appropriate culture medium, pH, temperature, and requirements for aerobic, microaerobic, or anaerobic conditions, depending on the specific requirements of the host cell, the fermentation, and the process.

The methods of producing isoprenoids provided herein may be performed in a suitable culture medium (e.g., with or without pantothenate supplementation) in a suitable container, including but not limited to a cell culture plate, a flask, or a fermentor. Further, the methods can be performed at any scale of fermentation known in the art to support industrial production of microbial products. Any suitable fermentor may be used including a stirred tank fermentor, an airlift fermentor, a bubble fermentor, or any combination thereof. In particular embodiments utilizing Saccharomyces cerevisiae as the host cell, strains can be grown in a fermentor as described in detail by Kosaric, et al, in Ullmann's Encyclopedia of Industrial Chemistry, Sixth Edition, Volume 12, pages 398-473, Wilcy-VCH Verlag GmbH & Co. KDaA, Weinheim, Germany.

In some embodiments, the culture medium is any culture medium in which a genetically modified microorganism capable of producing an isoprenoid can subsist, i.e., maintain growth and viability. In some embodiments, the culture medium is an aqueous medium comprising assimilable carbon, nitrogen and phosphate sources. Such a medium can also include appropriate salts, minerals, metals and other nutrients. In some embodiments, the carbon source and each of the essential cell nutrients, are added incrementally or continuously to the fermentation media, and each required nutrient is maintained at essentially the minimum level needed for efficient assimilation by growing cells, for example, in accordance with a predetermined cell growth curve based on the metabolic or respiratory function of the cells which convert the carbon source to a biomass.

Suitable conditions and suitable media for culturing microorganisms are well known in the art. In some embodiments, the suitable medium is supplemented with one or more additional agents, such as, for example, an inducer (e.g., when one or more nucleotide sequences encoding a gene product are under the control of an inducible promoter), a repressor (e.g., when one or more nucleotide sequences encoding a gene product are under the control of a repressible promoter), or a selection agent (e.g., an antibiotic to select for microorganisms comprising the genetic modifications).

In some embodiments, the carbon source is a monosaccharide (simple sugar), a disaccharide, a polysaccharide, a non-fermentable carbon source, or one or more combinations thereof. Non-limiting examples of suitable monosaccharides include glucose, galactose, mannose, fructose, xylose, ribose, and combinations thereof. Non-limiting examples of suitable disaccharides include sucrose, lactose, maltose, trehalose, cellobiose, and combinations thereof. Non-limiting examples of suitable polysaccharides include starch, glycogen, cellulose, chitin, and combinations thereof. Non-limiting examples of suitable non-fermentable carbon sources include acetate and glycerol.

The concentration of a carbon source, such as glucose, in the culture medium should promote cell growth, but not be so high as to repress growth of the microorganism used. Typically, cultures arc run with a carbon source, such as glucose, being added at levels to achieve the desired level of growth and biomass, but at undetectable levels (with detection limits being about <0.1 g/l). In other embodiments, the concentration of a carbon source, such as glucose, in the culture medium is greater than about 1 g/L, preferably greater than about 2 g/L, and more preferably greater than about 5 g/L. In addition, the concentration of a carbon source, such as glucose, in the culture medium is typically less than about 100 g/L, preferably less than about 50 g/L, and more preferably less than about 20 g/L. It should be noted that references to culture component concentrations can refer to both initial and/or ongoing component concentrations. In some cases, it may be desirable to allow the culture medium to become depleted of a carbon source during culture.

Sources of assimilable nitrogen that can be used in a suitable culture medium include, but are not limited to, simple nitrogen sources, organic nitrogen sources and complex nitrogen sources. Such nitrogen sources include anhydrous ammonia, ammonium salts and substances of animal, vegetable and/or microbial origin. Suitable nitrogen sources include, but are not limited to, protein hydrolysates, microbial biomass hydrolysates, peptone, yeast extract, ammonium sulfate, urea, and amino acids. Typically, the concentration of the nitrogen sources, in the culture medium is greater than about 0.1 g/L, preferably greater than about 0.25 g/L, and more preferably greater than about 1.0 g/L. Beyond certain concentrations, however, the addition of a nitrogen source to the culture medium is not advantageous for the growth of the microorganisms. As a result, the concentration of the nitrogen sources, in the culture medium is less than about 20 g/L, preferably less than about 10 g/L and more preferably less than about 5 g/L. Further, in some instances it may be desirable to allow the culture medium to become depleted of the nitrogen sources during culture.

The effective culture medium can contain other compounds such as inorganic salts, vitamins, trace metals or growth promoters. Such other compounds can also be present in carbon, nitrogen or mineral sources in the effective medium or can be added specifically to the medium.

The culture medium can also contain a suitable phosphate source. Such phosphate sources include both inorganic and organic phosphate sources. Preferred phosphate sources include, but are not limited to, phosphate salts such as mono or dibasic sodium and potassium phosphates, ammonium phosphate and mixtures thereof. Typically, the concentration of phosphate in the culture medium is greater than about 1.0 g/L, preferably greater than about 2.0 g/L and more preferably greater than about 5.0 g/L. Beyond certain concentrations, however, the addition of phosphate to the culture medium is not advantageous for the growth of the microorganisms. Accordingly, the concentration of phosphate in the culture medium is typically less than about 20 g/L, preferably less than about 15 g/L and more preferably less than about 10 g/L.

A suitable culture medium can also include a source of magnesium, preferably in the form of a physiologically acceptable salt, such as magnesium sulfate heptahydrate, although other magnesium sources in concentrations that contribute similar amounts of magnesium can be used. Typically, the concentration of magnesium in the culture medium is greater than about 0.5 g/L, preferably greater than about 1.0 g/L, and more preferably greater than about 2.0 g/L. Beyond certain concentrations, however, the addition of magnesium to the culture medium is not advantageous for the growth of the microorganisms. Accordingly, the concentration of magnesium in the culture medium is typically less than about 10 g/L, preferably less than about 5 g/L, and more preferably less than about 3 g/L. Further, in some instances it may be desirable to allow the culture medium to become depleted of a magnesium source during culture.

In some embodiments, the culture medium can also include a biologically acceptable chelating agent, such as the dihydrate of trisodium citrate. In such instance, the concentration of a chelating agent in the culture medium is greater than about 0.2 g/L, preferably greater than about 0.5 g/L, and more preferably greater than about 1 g/L. Beyond certain concentrations, however, the addition of a chelating agent to the culture medium is not advantageous for the growth of the microorganisms. Accordingly, the concentration of a chelating agent in the culture medium is typically less than about 10 g/L, preferably less than about 5 g/L, and more preferably less than about 2 g/L.

The culture medium can also initially include a biologically acceptable acid or base to maintain the desired pH of the culture medium. Biologically acceptable acids include, but are not limited to, hydrochloric acid, sulfuric acid, nitric acid, phosphoric acid and mixtures thereof. Biologically acceptable bases include, but are not limited to, ammonium hydroxide, sodium hydroxide, potassium hydroxide and mixtures thereof. In some embodiments, the base used is ammonium hydroxide.

The culture medium can also include a biologically acceptable calcium source, including, but not limited to, calcium chloride. Typically, the concentration of the calcium source, such as calcium chloride, dihydrate, in the culture medium is within the range of from about 5 mg/L to about 2000 mg/L, preferably within the range of from about 20 mg/L to about 1000 mg/L, and more preferably in the range of from about 50 mg/L to about 500 mg/L.

The culture medium can also include sodium chloride. Typically, the concentration of sodium chloride in the culture medium is within the range of from about 0.1 g/L to about 5 g/L, preferably within the range of from about 1 g/L to about 4 g/L, and more preferably in the range of from about 2 g/L to about 4 g/L.

In some embodiments, the culture medium can also include trace metals. Such trace metals can be added to the culture medium as a stock solution that, for convenience, can be prepared separately from the rest of the culture medium. Typically, the amount of such a trace metals solution added to the culture medium is greater than about 1 ml/L, preferably greater than about 5 mL/L, and more preferably greater than about 10 mL/L. Beyond certain concentrations, however, the addition of a trace metals to the culture medium is not advantageous for the growth of the microorganisms. Accordingly, the amount of such a trace metals solution added to the culture medium is typically less than about 100 mL/L, preferably less than about 50 mL/L, and more preferably less than about 30 mL/L. It should be noted that, in addition to adding trace metals in a stock solution, the individual components can be added separately, each within ranges corresponding independently to the amounts of the components dictated by the above ranges of the trace metals solution.

The culture media can include other vitamins, such as pantothenate, biotin, calcium, pantothenate, inositol, pyridoxine-HCl, and thiamine-HCl. Such vitamins can be added to the culture medium as a stock solution that, for convenience, can be prepared separately from the rest of the culture medium. Beyond certain concentrations, however, the addition of vitamins to the culture medium is not advantageous for the growth of the microorganisms.

The fermentation methods described herein can be performed in conventional culture modes, which include, but are not limited to, batch, fed-batch, cell recycle, continuous and semi-continuous. In some embodiments, the fermentation is carried out in fed-batch mode. In such a case, some of the components of the medium are depleted during culture, including pantothenate during the production stage of the fermentation. In some embodiments, the culture may be supplemented with relatively high concentrations of such components at the outset, for example, of the production stage, so that growth and/or isoprenoid production is supported for a period of time before additions are required. The preferred ranges of these components are maintained throughout the culture by making additions as levels are depleted by culture. Levels of components in the culture medium can be monitored by, for example, sampling the culture medium periodically and assaying for concentrations. Alternatively, once a standard culture procedure is developed, additions can be made at timed intervals corresponding to known levels at particular times throughout the culture. As will be recognized by those in the art, the rate of consumption of nutrient increases during culture as the cell density of the medium increases. Moreover, to avoid introduction of foreign microorganisms into the culture medium, addition is performed using aseptic addition methods, as are known in the art. In addition, a small amount of anti-foaming agent may be added during the culture.

The temperature of the culture medium can be any temperature suitable for growth of the genetically modified cells and/or production of isoprenoid. For example, prior to inoculation of the culture medium with an inoculum, the culture medium can be brought to and maintained at a temperature in the range of from about 20° C. to about 45° C., preferably to a temperature in the range of from about 25° C. to about 40° C., and more preferably in the range of from about 28° C. to about 32° C.

The pH of the culture medium can be controlled by the addition of acid or base to the culture medium. In such cases when ammonia is used to control pH, it also conveniently serves as a nitrogen source in the culture medium. Preferably, the pH is maintained from about 3.0 to about 8.0, more preferably from about 3.5 to about 7.0, and most preferably from about 4.0 to about 6.5.

In some embodiments, the carbon source concentration, such as the glucose concentration, of the culture medium is monitored during culture. Glucose concentration of the culture medium can be monitored using known techniques, such as, for example, use of the glucose oxidase enzyme test or high pressure liquid chromatography, which can be used to monitor glucose concentration in the supernatant, e.g., a cell-free component of the culture medium. As stated previously, the carbon source concentration should be kept below the level at which cell growth inhibition occurs. Although such concentration may vary from organism to organism, for glucose as a carbon source, cell growth inhibition occurs at glucose concentrations greater than at about 60 g/L, and can be determined readily by trial. Accordingly, when glucose is used as a carbon source the glucose is preferably fed to the fermentor and maintained below detection limits. Alternatively, the glucose concentration in the culture medium is maintained in the range of from about 1 g/L to about 100 g/L, more preferably in the range of from about 2 g/L to about 50 g/L, and yet more preferably in the range of from about 5 g/L to about 20 g/L. Although the carbon source concentration can be maintained within desired levels by addition of, for example, a substantially pure glucose solution, it is acceptable, and may be preferred, to maintain the carbon source concentration of the culture medium by addition of aliquots of the original culture medium. The use of aliquots of the original culture medium may be desirable because the concentrations of other nutrients in the medium (e.g. the nitrogen and phosphate sources) can be maintained simultaneously. Likewise, the trace metals concentrations can be maintained in the culture medium by addition of aliquots of the trace metals solution.

### 7.5 Recovery of Canthaxanthin

Once canthaxanthin is produced by the host cell, it may be recovered or isolated for subsequent use using any suitable separation and purification methods known in the art. In some embodiments, an aqueous phase comprising the canthaxanthin is separated from the fermentation by centrifugation. In other embodiments, an aqueous phase comprising the canthaxanthin is separated from the fermentation by adding a deemulsifier and/or a nucleating agent into the fermentation reaction. Illustrative examples of deemulsifiers include flocculants and coagulants. Illustrative examples of nucleating agents include droplets of the isoprenoid itself and organic solvents such as chloroform, ethyl acetate, acetone, hexane, ethanol, heptane, dodecane, isopropyl myristrate, and methyl oleate.

The canthaxanthin produced in these cells may be present in the culture supernatant and/or associated with the host cells. In embodiments where the isoprenoid is associated with the host cell, the recovery of the canthaxanthin may comprise a method of permeabilizing or lysing the cells. Alternatively or simultaneously, the canthaxanthin in the culture medium can be recovered using a recovery process including, but not limited to, chromatography, extraction, solvent extraction, membrane separation, electrodialysis, reverse osmosis, distillation, chemical derivatization and crystallization.

In some embodiments, the canthaxanthin is separated from other products that may be present. In some embodiments, the canthaxanthin is separated from other products that may be present in the aqueous phase. In some embodiments, the canthaxanthin is separated from other products that may be present in the organic phase. In some embodiments, separation is achieved using adsorption, distillation, gas-liquid extraction (stripping), liquid-liquid extraction (solvent extraction), ultrafiltration, and standard chromatographic techniques.

In some embodiments, the canthaxanthin produced in these cells may be present in the culture supernatant and the second isoprenoid produced in these cells may be associated with the host cells. The methods for separating and recovering the canthaxanthin, are described in the Example section below.

### 7.6 Methods of Making Genetically Modified Cells

Also provided herein are methods for producing a host cell that is genetically engineered to comprise one or more of the modifications described above, e.g., one or more heterologous nucleic acids encoding biosynthetic pathway enzymes, e.g., for co-production of isoprenoid compounds. Expression of a heterologous enzyme in a host cell can be accomplished by introducing into the host cells a nucleic acid comprising a nucleotide sequence encoding the enzyme under the control of regulatory elements that permit expression in the host cell. In some embodiments, the nucleic acid is an extrachromosomal plasmid. In other embodiments, the nucleic acid is a chromosomal integration vector that can integrate the nucleotide sequence into the chromosome of the host cell.

Nucleic acids encoding these proteins can be introduced into the host cell by any method known to one of skill in the art without limitation (see, for example, Hinnen et al. (1978) Proc. Natl. Acad. Sci. USA 75:1292-3; Cregg et al. (1985) Mol. Cell. Biol. 5:3376-3385; Goeddel et al. eds, 1990, Methods in Enzymology, vol. 185, Academic Press, Inc., CA; Krieger, 1990, Gene Transfer and Expression—A Laboratory Manual, Stockton Press, NY; Sambrook et al., 1989, Molecular Cloning—A Laboratory Manual, Cold Spring Harbor Laboratory, NY; and Ausubel et al., eds., Current Edition, Current Protocols in Molecular Biology, Greene Publishing Associates and Wiley Interscience, NY). Exemplary techniques include, but are not limited to, spheroplasting, electroporation, PEG 1000 mediated transformation, and lithium acetate or lithium chloride mediated transformation.

The copy number of an enzyme in a host cell may be altered by modifying the transcription of the gene that encodes the enzyme. This can be achieved for example by modifying the copy number of the nucleotide sequence encoding the enzyme (e.g., by using a higher or lower copy number expression vector comprising the nucleotide sequence, or by introducing additional copies of the nucleotide sequence into the genome of the host cell or by deleting or disrupting the nucleotide sequence in the genome of the host cell), by changing the order of coding sequences on a polycistronic mRNA of an operon or breaking up an operon into individual genes each with its own control elements, or by increasing the strength of the promoter or operator to which the nucleotide sequence is operably linked. Alternatively or in addition, the copy number of an enzyme in a host cell may be altered by modifying the level of translation of an mRNA that encodes the enzyme. This can be achieved for example by modifying the stability of the mRNA, modifying the sequence of the ribosome binding site, modifying the distance or sequence between the ribosome binding site and the start codon of the enzyme coding sequence, modifying the entire intercistronic region located “upstream of” or adjacent to the 5′ side of the start codon of the enzyme coding region, stabilizing the 3′-end of the mRNA transcript using hairpins and specialized sequences, modifying the codon usage of enzyme, altering expression of rare codon tRNAs used in the biosynthesis of the enzyme, and/or increasing the stability of the enzyme, as, for example, via mutation of its coding sequence.

The activity of an enzyme in a host cell can be altered in a number of ways, including, but not limited to, expressing a modified form of the enzyme that exhibits increased or decreased solubility in the host cell, expressing an altered form of the enzyme that lacks a domain through which the activity of the enzyme is inhibited, expressing a modified form of the enzyme that has a higher or lower Kcat or a lower or higher Km for the substrate, or expressing an altered form of the enzyme that is more or less affected by feed-back or feed-forward regulation by another molecule in the pathway.

In some embodiments, a nucleic acid used to genetically modify a host cell comprises one or more selectable markers useful for the selection of transformed host cells and for placing selective pressure on the host cell to maintain the foreign DNA.

In some embodiments, the selectable marker is an antibiotic resistance marker. Illustrative examples of antibiotic resistance markers include, but are not limited to, the BLA, NAT1, PAT, AUR1-C, PDR4, SMR1, CAT, mouse dhfr, HPH, DSDA, KANR, and SH BLE gene products. The BLA gene product from E. coli confers resistance to beta-lactam antibiotics (e.g., narrow-spectrum cephalosporins, cephamycins, and carbapenems (ertapenem), cefamandole, and cefoperazone) and to all the anti-gram-negative-bacterium penicillins except temocillin; the NAT1 gene product from S. noursei confers resistance to nourseothricin; the PAT gene product from S. viridochromogenes Tu94 confers resistance to bialophos; the AUR1-C gene product from Saccharomyces cerevisiae confers resistance to Auerobasidin A (AbA); the PDR4 gene product confers resistance to cerulenin; the SMR1 gene product confers resistance to sulfometuron methyl; the CAT gene product from Tn9 transposon confers resistance to chloramphenicol; the mouse dhfr gene product confers resistance to methotrexate; the HPH gene product of Klebsiella pneumonia confers resistance to Hygromycin B; the DSDA gene product of E. coli allows cells to grow on plates with D-serine as the sole nitrogen source; the KANR gene of the Tn903 transposon confers resistance to G418; and the SH BLE gene product from Streptoalloteichus hindustanus confers resistance to Zeocin (bleomycin). In some embodiments, the antibiotic resistance marker is deleted after the genetically modified host cell disclosed herein is isolated.

In some embodiments, the selectable marker rescues an auxotrophy (e.g., a nutritional auxotrophy) in the genetically modified microorganism. In such embodiments, a parent microorganism comprises a functional disruption in one or more gene products that function in an amino acid or nucleotide biosynthetic pathway and that when non-functional renders a parent cell incapable of growing in media without supplementation with one or more nutrients. Such gene products include, but are not limited to, the HIS3, LEU2, LYS1, LYS2, MET15, TRP1, ADE2, and URA3 gene products in yeast. The auxotrophic phenotype can then be rescued by transforming the parent cell with an expression vector or chromosomal integration construct encoding a functional copy of the disrupted gene product, and the genetically modified host cell generated can be selected for based on the loss of the auxotrophic phenotype of the parent cell. Utilization of the URA3, TRP1, and LYS2 genes as selectable markers has a marked advantage because both positive and negative selections are possible. Positive selection is carried out by auxotrophic complementation of the URA3, TRP 1, and LYS2 mutations, whereas negative selection is based on specific inhibitors, i.e., 5-fluoro-orotic acid (FOA), 5-fluoroanthranilic acid, and aminoadipic acid (aAA), respectively. that prevent growth of the prototrophic strains but allows growth of the URA3, TRP1, and LYS2 mutants, respectively. In other embodiments, the selectable marker rescues other non-lethal deficiencies or phenotypes that can be identified by a known selection method.

Described herein are specific genes and proteins useful in the methods, compositions and organisms of the disclosure; however it will be recognized that absolute identity to such genes is not necessary. For example, changes in a particular gene or polynucleotide comprising a sequence encoding a polypeptide or enzyme can be performed and screened for activity. Typically such changes comprise conservative mutations and silent mutations. Such modified or mutated polynucleotides and polypeptides can be screened for expression of a functional enzyme using methods known in the art.

Due to the inherent degeneracy of the genetic code, other polynucleotides which encode substantially the same or functionally equivalent polypeptides can also be used to clone and express the polynucleotides encoding such enzymes.

As will be understood by those of skill in the art, it can be advantageous to modify a coding sequence to enhance its expression in a particular host. The genetic code is redundant with 64 possible codons, but most organisms typically use a subset of these codons. The codons that are utilized most often in a species are called optimal codons, and those not utilized very often are classified as rare or low-usage codons. Codons can be substituted to reflect the preferred codon usage of the host, in a process sometimes called “codon optimization” or “controlling for species codon bias.”

Optimized coding sequences containing codons preferred by a particular prokaryotic or eukaryotic host (Murray et al., 1989, Nucl Acids Res. 17:477-508) can be prepared, for example, to increase the rate of translation or to produce recombinant RNA transcripts having desirable properties, such as a longer half-life, as compared with transcripts produced from a non-optimized sequence. Translation stop codons can also be modified to reflect host preference. For example, typical stop codons for S. cerevisiae and mammals are UAA and UGA, respectively. The typical stop codon for monocotyledonous plants is UGA, whereas insects and E. coli commonly use UAA as the stop codon (Dalphin et al., 1996, Nucl Acids Res. 24:216-8).

Those of skill in the art will recognize that, due to the degenerate nature of the genetic code, a variety of DNA molecules differing in their nucleotide sequences can be used to encode a given enzyme of the disclosure. The native DNA sequence encoding the biosynthetic enzymes described above are referenced herein merely to illustrate an embodiment of the disclosure, and the disclosure includes DNA molecules of any sequence that encode the amino acid sequences of the polypeptides and proteins of the enzymes utilized in the methods of the disclosure. In similar fashion, a polypeptide can typically tolerate one or more amino acid substitutions, deletions, and insertions in its amino acid sequence without loss or significant loss of a desired activity. The disclosure includes such polypeptides with different amino acid sequences than the specific proteins described herein so long as the modified or variant polypeptides have the enzymatic anabolic or catabolic activity of the reference polypeptide. Furthermore, the amino acid sequences encoded by the DNA sequences shown herein merely illustrate embodiments of the disclosure.

In addition, homologs of enzymes useful for the compositions and methods provided herein are encompassed by the disclosure. In some embodiments, two proteins (or a region of the proteins) are substantially homologous when the amino acid sequences have at least about 30%, 40%, 50%, 60%, 65%, 70%, 75%, 80%, 85%, 90%, 91%, 92%, 93%, 94%, 95%, 96%, 97%, 98%, or 99% identity. To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In one embodiment, the length of a reference sequence aligned for comparison purposes is at least 30%, typically at least 40%, more typically at least 50%, even more typically at least 60%, and even more typically at least 70%, 80%, 90%, 100% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences, taking into account the number of gaps, and the length of each gap, which need to be introduced for optimal alignment of the two sequences.

When “homologous” is used in reference to proteins or peptides, it is recognized that residue positions that are not identical often differ by conservative amino acid substitutions. A “conservative amino acid substitution” is one in which an amino acid residue is substituted by another amino acid residue having a side chain (R group) with similar chemical properties (e.g., charge or hydrophobicity). In general, a conservative amino acid substitution will not substantially change the functional properties of a protein. In cases where two or more amino acid sequences differ from each other by conservative substitutions, the percent sequence identity or degree of homology may be adjusted upwards to correct for the conservative nature of the substitution. Means for making this adjustment are well known to those of skill in the art (See, e.g., Pearson W. R., 1994, Methods in Mol Biol 25:365-89).

The following six groups each contain amino acids that are conservative substitutions for one another: 1) Serine(S), Threonine (T); 2) Aspartic Acid (D), Glutamic Acid (E); 3) Asparagine (N), Glutamine (Q); 4) Arginine (R), Lysine (K); 5) Isoleucine (I), Leucine (L), Alanine (A), Valine (V), and 6) Phenylalanine (F), Tyrosine (Y), Tryptophan (W).

Sequence homology for polypeptides, which is also referred to as percent sequence identity, is typically measured using sequence analysis software. A typical algorithm used comparing a molecule sequence to a database containing a large number of sequences from different organisms is the computer program BLAST. When searching a database containing sequences from a large number of different organisms, it is typical to compare amino acid sequences.

Furthermore, any of the genes encoding the foregoing enzymes (or any others mentioned herein (or any of the regulatory elements that control or modulate expression thereof)) may be optimized by genetic/protein engineering techniques, such as directed evolution or rational mutagenesis, which are known to those of ordinary skill in the art. Such action allows those of ordinary skill in the art to optimize the enzymes for expression and activity in yeast.

In addition, genes encoding these enzymes can be identified from other fungal and bacterial species and can be expressed for the modulation of this pathway. A variety of organisms could serve as sources for these enzymes, including, but not limited to, Saccharomyces spp., including S. cerevisiae and S. uvarum, Kluyveromyces spp., including K. thermotolerans, K. lactis, and K. marxianus, Pichia spp., Hansenula spp., including H. polymorpha, Candida spp., Trichosporon spp., Yamadazyma spp., including Y. spp. stipitis, Torulaspora pretoriensis, Issatchenkia orientalis, Schizosaccharomyces spp., including S. pombe, Cryptococcus spp., Aspergillus spp., Neurospora spp., or Ustilago spp. Sources of genes from anaerobic fungi include, but are not limited to, Piromyces spp., Orpinomyces spp., or Neocallimastix spp. Sources of prokaryotic enzymes that are useful include, but are not limited to, Escherichia. coli, Zymomonas mobilis, Staphylococcus aureus, Bacillus spp., Clostridium spp., Corynebacterium spp., Pseudomonas spp., Lactococcus spp., Enterobacter spp., Salmonella spp., or X. dendrorhous

Techniques known to those skilled in the art may be suitable to identify additional homologous genes and homologous enzymes. Generally, analogous genes and/or analogous enzymes can be identified by functional analysis and will have functional similarities. Techniques known to those skilled in the art may be suitable to identify analogous genes and analogous enzymes. For example, to identify homologous or analogous PK, PTA, RHR2, HOR2, or carotenogic genes, proteins, or enzymes, techniques may include, but are not limited to, cloning a gene by PCR using primers based on a published sequence of a gene/enzyme of interest, or by degenerate PCR using degenerate primers designed to amplify a conserved region among a gene of interest. Further, one skilled in the art can use techniques to identify homologous or analogous genes, proteins, or enzymes with functional homology or similarity. Techniques include examining a cell or cell culture for the catalytic activity of an enzyme through in vitro enzyme assays for said activity (e.g. as described herein or in Kiritani, K., Branched-Chain Amino Acids Methods Enzymology, 1970), then isolating the enzyme with said activity through purification, determining the protein sequence of the enzyme through techniques such as Edman degradation, design of PCR primers to the likely nucleic acid sequence, amplification of said DNA sequence through PCR, and cloning of said nucleic acid sequence. To identify homologous or similar genes and/or homologous or similar enzymes, analogous genes and/or analogous enzymes or proteins, techniques also include comparison of data concerning a candidate gene or enzyme with databases such as BRENDA, KEGG, or MetaCYC. The candidate gene or enzyme may be identified within the above mentioned databases in accordance with the teachings herein.

### 8. EXAMPLES

### 8.1 Canthaxanthin Production Strains

The present example provides the production of strains that convert β-carotene to canthaxanthin at high yields and efficiencies.

### 8.1.1 Strain Descriptions

Strains shown in Table 6-1 were derived from strain Y227 as previously described (Westfall et al., 2012, Proceedings of the National Academy of Sciences 109, E111-E118; Meadows et al., 2016, Nature 537, 694-697). The farnesene manufacturing strain AMR-6 has a genotype highly similar to strain AMR-5 which has been extensively described (Meadows et al., 2016, Nature 537, 694-697). DNA constructs were integrated in intergenic regions of the genome (except at GAS4 locus where the GAS4 gene was deleted), at the downstream of a given gene (denoted by * to represent the stop codon of the gene).

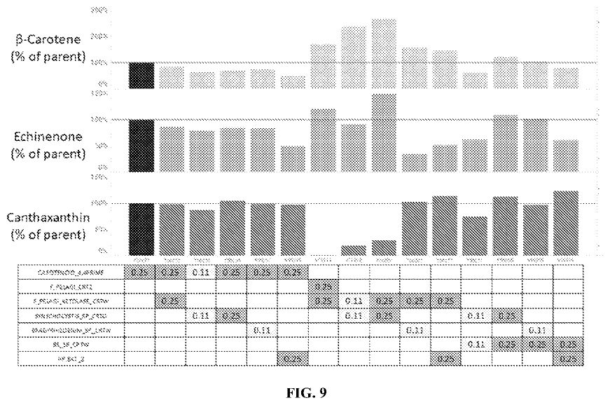

TABLE 6-1
Genotypes of S. cerevisiae strains. Strains are also listed in FIG. 8.

| | BASE | | |
|---|---|---|---|
| STRAIN | STRAIN | GENOTYPES | SOURCE |
| Y35965 | | Similar to Dalton (Y27011) | Meadows |
| (=AMR-6) | | | |
| Y47245 | Y35965 | gas4Δ: 0.63 × PGAL1 > Xd.CrtI_tQCR10::0.25 × | |
| (=AMR-12) | (=AMR-6) | PGAL1 > Xd.CrtYB_tNCE103 | |
| | | NOP4[*425-*826]Δ: 0.26 × PGAL1 > BTS1 | |
| Y47247 | Y35965 | gas4Δ: 0.61 × PGAL1 > Xd.CrtI_tQCR10::0.25 × | |
| (=AMR-13) | (=AMR-6) | PGAL1 > Xd.CrtYB_tNCE103 | |
| | | NOP4[*425-*826]Δ: 0.21 × PGAL1 > BTS1 | |
| Y51702 | AMR-13 | ALG1[*213-*788]Δ: 0.54 × PGAL1 > Pa.CrtW_tPGK1 | |
| (=AMR-16) | | | |
| Y51703 | AMR-13 | ALG1[*213-*788]Δ: 0.37 × PGAL1 > Pa.CrtW_tPGK1 | |
| (=AMR-17) | | | |
| Y51700 | AMR-13 | ALG1[*213-*788]Δ: 0.36 × PGAL1 > Pa.CrtW_tPGK1 | |
| (=AMR-18) | | | |
| Y51704 | AMR-13 | ALG1[*213-*788]Δ: 0.36 × PGAL1 > Pa.CrtW_tPGK1 | |
| (=AMR-19) | | | |
| Y51707 | AMR-13 | ALG1[*213-*788]Δ: 0.36 × PGAL1 > Pa.CrtW_tPGK1 | |
| (=AMR-20) | | | |
| Y51699 | AMR-13 | ALG1[*213-*788]Δ: 0.35 × PGAL1 > Pa.CrtW_tPGK1 | |
| (=AMR-21) | | | |
| Y51701 | AMR-13 | ALG1[*213-*788]Δ: 0.32 × PGAL1 > Pa.CrtW_tPGK1 | |
| (=AMR-22) | | | |
| Y51691 | AMR-13 | ALG1[*213-*788]Δ: 0.25 × PGAL1 > Pa.CrtW_tPGK1 | |
| (=AMR-23) | | | |
| Y51692 | AMR-13 | ALG1[*213-*788]Δ: 0.21 × PGAL1 > Pa.CrtW_tPGK1 | |
| (=AMR-24) | | | |
| Y51705 | AMR-13 | ALG1[*213-*788]Δ: 0.11 × PGAL1 > Pa.CrtW_tPGK1 | |
| (=AMR-25) | | | |
| Y51706 | AMR-13 | ALG1[*213-*788]Δ: 0.05 × PGAL1 > Pa.CrtW_tPGK1 | |
| (=AMR-26) | | | |
| AMR-27 | AMR-13 | ALG1[*213-*788]Δ: 0.25 × PGAL1 > Bradyrhizobium | |
| | | sp.CrtW_tHUG1 | |
| AMR-28 | AMR-13 | ALG1[*213-*788]Δ: 0.25 × PGAL1 > Pa.CrtW_tPGK1::0.11 × | |
| | | PGAL1 > Bradyrhizobium sp.CrtW_tHUG1 | |

### 8.1.2 DNA Assembly and Yeast Transformation

Multicomponent DNA constructs were generated using assembly methods as previously described (Kok et al., 2014, ACS Synthetic Biology 3, 97-106; Serber et al., 2012, COMPOSITIONS AND METHODS FOR THE ASSEMBLY OF POLYNUCLEOTDES; Westfall ct al., 2012).

### 8.1.3 Media and Strain Cultivation in 96-Well Microtiter Plates

YP, CSM, and agar plates were previously described (Westfall et al., 2012). Production of carotenoids was conducted in 96-well microtiter plate at 1,000 rpm shaking with 80% relative humidity (Infors Multitron with 3 mm throw). Cultures used for the inoculation of the microtiter plates were maintained on YP medium agar plates with 2% glucose, 1% maltose, 2 g/L Lysine and 50 μg/mL G418 or nourseothricin at 28° C. Colonies were picked into sterile, 96-well microtiter plates (1.1 mL working volume; Axygen) containing 360 μL of defined liquid growth bird seed media (BSM) with 2% total carbon (1.4% sucrose, 0.7% maltose) and 1 g/L lysine. This plate was incubated for 72 hours at 28° C., 80% humidity and 1000 rpm. The initial biomass build was diluted 1/25 into two sterile 1.1 mL or 2.2 mL plates containing 360 μL of defined liquid growth bird seed media (BSM) containing 4% sucrose as a carbon source. One plate was used to measure OD₆₀₀, C40-carotenoids, and protein expression. The second was used to measure farnesene production. Both production plates were incubated at 33.5° C. (Meadows et al., 2016)

### 8.1.4 C40 Carotenoids Extraction and Measurements

C40 carotenoids were extracted from 96-well microtiter plates using dimethyl sulfoxide (DMSO) to disrupt yeast cells followed by extraction of C40 carotenoids using heptane. After growth for three days in 2.2 mL 96-well microtiter plate, as described above, the biomass was harvested by centrifugation for 5 min @4250 rpm, 20° C. in a Sorvall HT6 centrifuge (ThermoFisher) to pellet cells. After removing the supernatant, 600 μL of DMSO was added to the cell pellet and the plate was sealed using PlateLoc aluminum seal and was shaken using MixMate (Eppendorf, Hamburg, Germany) at 1500 rpm for 30 minutes. The seal was then removed, 600 μL of heptane was added, and the plate was sealed and shaken again for an additional 30 minutes at 1500 rpm. 50 μL of PBS was added to each well, the plate was shaken for 5 more minutes, and centrifuged for 5 min @4250 rpm, 20° C. in Sorvall HT6 centrifuge to settle the biphasic layers. 200 μL of the top heptane layer was transferred to a 1.1 mL Axygen round-bottom plate after which the sample was submitted for chromatographic analysis.

C40 carotenoids were extracted from bioreactor broth samples after a 200 μL of well-vortexed sample was transferred to a 2 mL screw cap tube and centrifuged for 2 min at full speed using an Eppendorf 5452 Minispin Centrifuge (Eppendorf, Hamburg, Germany). The supernatant was discarded and one scoop of 1 mm glass beads (Mini-BeadBeater Glass Mill Beads, Biospec Products, Bartlesville, OK), was added to the pellet using a bead scoop. The tube was placed on MP Fastprep 24 Bead Beater (MP Biomedicals) and, after 600 μL of chloroform was added to the sample tube, the instrument was set at 6.5 m/s for 1 minute. After letting the sample cool down on ice for 5 minutes, the process of bead beating was repeated one more time. The sample was then vortexed at 1500 rpm for 30 minutes, allowed to settle, and 400 μL of the chloroform layer was transferred to a vial and submitted for chromatographic analysis.

C40 carotenoids were chromatographically analyzed using UV-UPLC. For each sample, 2 μL was injected on to a Zorbax Eclipse Plus C8 1.8 μm 100 mm×2.1 mm column (Agilent, Santa Clara, CA) at an initial flow rate of 0.4 mL/min, ramping to 0.5 ml/min at 1 min. Separate gradients were used for analysis, using mobile phase A (50% methanol in H₂O, 0.1% Acetic Acid, 5 mM Ammonium Acetate) and mobile phase B (1:8:1 Methanol:IPA:H₂O, 0.1% Acetic Acid, 5 mM Ammonium Acetate), as follows: Gradient started at 2% B, ramping to 35% B at 1 min, 50% B at 1.5 min, 60% B at 2 min, and held until 5 min. At 5 min, the gradient was stepped up to 90% B and held until 8 min. It is then ramped to 95% B at 8.25 min, then reduced to 2% B at 8.5 min and held until the end of the run. C40 carotenoids were detected using a Vanquish Variable Wavelength Detector VF-D40/Vanquish Diode Array Detector VH-D10-A with the detector wavelength set at 250 nm, 471 nm, and 454 nm. A solution of standards was used to check retention time. Peak heights were converted to concentration values from external standard calibrations using standard compounds. Phytoene was quantitated using 250 nm, lycopene using 471 nm, □-carotene and canthaxanthin using 454 nm. All data were plotted using TIBCO Spotfire Data Visualization and Analytics Software.

### 8.1.5 Protein Measurements and Analysis Using LC-MS/MS

Strains were grown in 2.2 mL 96-well microtiter plates from a single colony with four biological replicates. After three days of incubation, as described above, the biomass was harvested by centrifugation using Allegra X-30 centrifuge (Beckman Coulter) at 1,000×g for 5 min. After removing the supernatant, the cell pellet was washed once with 500 μL of 50% ethanol and twice with 500 μL of sterile water. The cell pellet was then resuspended in 160 μL of ice cold 100 mM ammonium bicarbonate buffer pH 8.5 with cOmplete™, Mini EDTA-free Protease Inhibitor Cocktail (Roche). Cells were lysed by bead-beating using Minibeadbeter-96 (Biospec) for 3 minutes at maximum speed. The samples were boiled at 99° C. for 10 minutes after adding 40 μL of 10% SDS. Then 50 μL of 5 M urea in 0.1 M ammonium bicarbonate was added and SDS was removed using Pierce™ detergent removal spin plates (ThermoFisher). Total protein was measured using BCA assay (Pierce™ BCA Protein Assay Kit, ThermoFisher) with BSA as a standard. Protein lysates were then normalized to a total of 200 μg protein in 200 μL. Trypsin (Sigma) was added at a 1:20 ratio (10 μg Trypsin) and samples were digested at 37° C. for 4 h. After the end of trypsin digestion, samples were quenched using 0.1% formic acid.

The samples were analyzed using a quadrupole-orbitrap mass spectrometer (Q-Exactive, Thermo Scientific) equipped with an UPLC separation system (Vanquish, Thermo Scientific). Each sample containing 10 μg of total protein was separated on the Acquity BEH 300 C18 column (2.1 mm×50 mm, 1.7 μm, Waters) at 30° C. Gradient elution was performed with a mixture of 0.1% formic acid-water (mobile phase A) and 0.1% formic acid-acetonitrile (mobile phase B) at the flow rate of 0.5 mL/min: 2% B for 0 min to 2 min; 2% B to 35% B for 2 min to 27 min; 95% B for 27 min to 31 min; and 2% B for 31 min to 35 min. For the mass spectrometry (MS) analysis, ionization conditions were set at the sheath gas flow rate of 40 L/min, aux gas flow rate of 10 L/min, spray voltage of 3.5 kV, capillary temperature of 320° C., s-lens RF level of 50, and aux gas heater temperature of 350° C. The resolution was set to 17,500, the AGC target value at 1×10⁵, isolation width at 4.0 m/z, and injection time were set at 30 ms. The normalized collision energy was set at 27 eV. Parallel-reaction monitoring (PRM) experiments were performed using a scheduled (2 min window) transition list generated in Skyline that consisted of m/z of precursor peptides of interest and corresponding retention times. Skyline software was used for PRM data analysis. The relative quantification was performed using the sum of peak areas obtained for 3-5 transitions of the representative peptide for each protein and the results were reported as the peak area ratio to the area of ACTI peptide. The results were plotted using TIBCO Spotfire Data Visualization and Analytics Software.

### 8.1.6 Cultivation in Bioreactors for Farnesene and C40 Carotenoid Co-Production

The fermentation process used here utilizes cane syrup as a feedstock in a fed-batch process controlled by a custom feeding algorithm essentially as described. (Meadows et al., 2016) The fermentation algorithm is designed to minimize ethanol formation, but maximize nutrient utilization rates (glucose and O₂) by means of cycling between two feed rates: (1) a 2-5 h period at ‘max sugar feed’ (~10-20 g/L/h, controlled in a dynamic way by “on-demand” feedback that changes depending on the oxygen transfer rate and strain), which results in full O₂ utilization and a small amount of ethanol formation, and (2) a 15-60 minute period at a ‘min sugar feed’ (either 1 g/L/h or 50% of max feed rate), which allows for re-consumption of the produced ethanol while co-consuming a slow sugar feed. The high feed rate resumes when the dissolved oxygen spikes, indicating the exhaustion of residual carbon; the high feed rate ends after a set amount of sugar (grams per liter of broth) is added. At the end of every day in the 8-day process, all 0.5 L tanks had broth drawn off until the tank contained a set working volume (200 mL). All fermentations were done at 30° C.

### 8.1.7 Identification of Echinenone Peak Using Mass Spectroscopy

For each sample, 3.0 μL was injected on to an ACQUITY UPLC BEH C18 Column, 130 Å, 1.7 μm, 2.1 mm×50 mm at a flow rate of 0.400 mL/min. The solvents used were: Solvent A (50% Methanol, 50% Water) and Solvent B (90% Isopropanol, 10% Water). The gradient used was as follows: gradient started at 90% A and held for 0.5 min, ramped from 10% to 100% B over 10 min, held at 100% B for 2 min, ramped to 10% B over 0.1 min and then held at 10% B for 2.4 min. Mass spec was acquired using an Agilent MS-QTOF (G6545A) equipped with an Atmospheric Pressure Chemical Ionization Source (APCI) (G1947B) optimized for a flow rate of 0.400 mL/min as follows: Gas Temp (° C.)=300, Vaporizer Temp (° C.)=300, Gas Flow (l/min)=5, Nebulizer (psig)=40, VCap (V)=3000, Corona Positive=4, Fragmentor (V)=140, Skimmer1 (V)=65, OctopoleRFPeak=750, MassCalibrationRange=50-1700 m/z were used for each analyte. Analyte signal was integrated using Agilent MassHunter Qualitative Analysis B.07.00.

### 8.2 Results

FIG. 1 provides a pathway for production of β-carotene and canthaxanthin showing the enzymes and genes described below and the species from which the genes are derived.

8.2.1 Promoter Strengths as Fractions of the Strong Yeast GAL1 Promoter (pGAL1):

Overexpression of mevalonate and carotenoid pathway genes use the native GAL regulon promoters such as pGAL1/10 (Meadows et al., 2016), but galactose is not needed for induction since AMR-6 utilizes a modified GAL regulon that is repressed by maltose instead of activated by galactose (Chua et al., 2020, U.S. Pat. No. 10,808,015 B2). In addition, a library of synthetic and natural pGAL promoters was created and used as described. (Deng et al., 2021, Microbial Cell Factories 2, 202). This ladder of pGAL promoters ranges in strength from 1-100% of the strong GAL1 promoter (ranked by GFP production in a wild-type strain), allowing modulated expression of carotenoid pathway genes. Promoter strengths are described below as multiples of pGAL1 strength. e.g., 0.21×pGAL1.

### 8.2.2 Parental β-Carotene Production Strain

The β-carotene production strain was Y47247, which produces both β-carotene and farnesene.

Strain Y47247 was derived from strain Y35965, which is similar to Dalton (Y27011), and produces farnesene as described. (Meadows et al., 2016) Additional engineering to enable production of β-carotene was as follows (all heterologous genes are codon-optimized for S. cerevisiae expression):

BTS1 was expressed from 0.21×pGAL1, which is in addition to the native BTS1 gene. BTS1 is the yeast gene encoding Geranylgeranyl diphosphate synthase (GGPPS; yeastgenome.org/locus/S000005990; SEQ ID NO:5 nucleotide and SEQ ID NO:6 protein). The yeast gene encoding Geranylgeranyl diphosphate synthase (GGPPS, yeastgenome.org/locus/S000005990; SEQ ID NO:5):

ATGGAGGCCAAGATAGATGAGCTGATCAATAATGATCCTGTTTGGTCC
AGCCAAAATGAAAGCTTGATTTCAAAACCTTATAATCACATCCTTTTG
AAACCTGGCAAGAACTTTAGACTAAATTTAATAGTTCAAATTAACAGA
GTTATGAATTTGCCCAAAGACCAGCTGGCCATAGTTTCGCAAATTGTT
GAGCTCTTGCATAATTCCAGCCTTTTAATCGACGATATAGAAGATAAT
GCTCCCTTGAGAAGGGGACAGACCACTTCTCACTTAATCTTCGGTGTA
CCCTCCACTATAAACACCGCAAATTATATGTATTTCAGAGCCATGCAA
CTTGTATCGCAGCTAACCACAAAAGAGCCTTTGTATCATAATTTGATT
ACGATTTTCAACGAAGAATTGATCAATCTACATAGGGGACAAGGCTTG
GATATATACTGGAGAGACTTTCTGCCTGAAATCATACCTACTCAGGAG
ATGTATTTGAATATGGTTATGAATAAAACAGGCGGCCTTTTCAGATTA
ACGTTGAGACTCATGGAAGCGCTGTCTCCTTCCTCACACCACGGCCAT
TCGTTGGTTCCTTTCATAAATCTTCTGGGTATTATTTATCAGATTAGA
GATGATTACTTGAATTTGAAAGATTTCCAAATGTCCAGCGAAAAAGGC
TTTGCTGAGGACATTACAGAGGGGAAGTTATCTTTTCCCATCGTCCAC
GCCCTTAACTTCACTAAAACGAAAGGTCAAACTGAGCAACACAATGAA
ATTCTAAGAATTCTCCTGTTGAGGACAAGTGATAAAGATATAAAACTA
AAGCTGATTCAAATACTGGAATTCGACACCAATTCATTGGCCTACACC
AAAAATTTTATTAATCAATTAGTGAATATGATAAAAAATGATAATGAA
AATAAGTATTTACCTGATTTGGCTTCGCATTCCGACACCGCCACCAAT
TTACATGACGAATTGTTATATATAATAGACCACTTATCCGAATTGTGA

The yeast Geranylgeranyl diphosphate synthase (GGPPS, yeastgenome.org/locus/S000005990; SEQ ID NO: 6):

MEAKIDELINNDPVWSSQNESLISKPYNHILLKPGKNFRLNLIVQINR
VMNLPKDQLAIVSQIVELLHNSSLLIDDIEDNAPLRRGQTTSHLIFGV
PSTINTANYMYFRAMQLVSQLTTKEPLYHNLITIFNEELINLHRGQGL
DIYWRDFLPEIIPTQEMYLNMVMNKTGGLFRLTLRLMEALSPSSHHGH
SLVPFINLLGIIYQIRDDYLNLKDFQMSSEKGFAEDITEGKLSFPIVH
ALNFTKTKGQTEQHNEILRILLLRTSDKDIKLKLIQILEFDTNSLAYT
KNFINQLVNMIKNDNENKYLPDLASHSDTATNLHDELLYIIDHLSEL*

Xanthophyllomyces dendrorhous CrtYB gene was expressed from 0.25×pGAL1. The terminator on CrtYB was tNCE103. CrtYB encodes a bifunctional enzyme with both phytoene synthase and lycopene cyclase activities. (uniprot, org/uniprotkb/Q7Z859/entry) The DNA sequence of yeast codon-optimized XdCrtYB, including NCI terminator (SEQ ID NO:7):

ATGACCGCCTTGGCCTACTATCAAATTCATTTGATTTACACTTTGCCA
ATCTTGGGTTTGTTGGGTTTATTGACTTCTCCAATTTTAACTAAGTTC
GACATCTACAAGATCTCTATTTTGGTTTTCATTGCCTTTTCTGCTACT
ACTCCATGGGATTCTTGGATTATTCGTAACGGTGCTTGGACTTACCCA
TCTGCTGAATCCGGTCAAGGTGTTTTTGGTACTTTTTTGGACGTTCCA
TACGAAGAATACGCCTTTTTCGTTATCCAAACTGTTATCACCGGTTTG
GTCTACGTCTTGGCCACTAGACACTTGTTACCATCTTTGGCTTTACCA
AAAACTAGATCCTCTGCTTTGTCTTTAGCCTTGAAGGCCTTGATTCCA
TTGCCAATTATCTATTTGTTTACCGCTCATCCATCTCCATCTCCAGAT
CCATTGGTCACCGACCACTACTTCTACATGAGAGCTTTGTCTTTATTG
ATCACTCCTCCAACTATGTTGTTGGCCGCTTTGTCCGGTGAATATGCT
TTCGATTGGAAGTCTGGTAGAGCCAAATCTACCATCGCTGCCATTATG
ATTCCTACTGTCTACTTGATCTGGGTCGACTACGTTGCCGTTGGTCAA
GATTCCTGGTCTATTAATGATGAAAAGATCGTTGGTTGGCGTTTGGGT
GGTGTTTTGCCAATTGAAGAAGCTATGTTCTTCTTGTTGACCAACTTG
ATGATTGTCTTAGGTTTATCCGCTTGTGACCATACTCAAGCTTTGTAC
TTGTTGCACGGTCGTACTATCTACGGTAACAAGAAAATGCCATCCTCC
TTCCCATTGATTACCCCACCAGTCTTGTCTTTGTTCTTCTCTTCTAGA
CCATACTCTTCCCAACCAAAGAGAGACTTAGAGTTAGCTGTTAAGTTG
TTAGAAGAAAAGTCCAGATCTTTCTTCGTCGCTTCCGCTGGTTTTCCA
TCCGAGGTTAGAGAACGTTTGGTTGGTTTGTACGCCTTCTGTAGAGTC
ACCGATGATTTGATTGACTCTCCAGAGGTTTCTTCCAATCCACACGCC
ACCATCGACATGGTTTCCGATTTCTTGACCTTGTTGTTCGGTCCACCA
TTACACCCATCTCAACCAGACAAGATTTTGTCTTCTCCTTTGTTGCCA
CCATCCCACCCATCTAGACCAACCGGTATGTATCCATTGCCACCACCA
CCATCTTTGTCTCCAGCTGAATTGGTTCAATTCTTAACTGAAAGAGTC
CCTGTTCAATATCACTTTGCTTTTAGATTGTTGGCCAAGTTACAAGGT
TTGATTCCAAGATACCCATTGGACGAATTGTTGAGAGGTTACACTACC
GATTTAATCTTCCCATTGTCTACTGAAGCTGTTCAAGCCAGAAAGACC
CCAATTGAAACTACCGCTGATTTATTGGACTACGGTTTATGTGTCGCC
GGTTCTGTTGCTGAATTATTGGTCTACGTTTCTTGGGCTTCCGCTCCT
TCCCAAGTTCCAGCTACTATTGAAGAAAGAGAGGCTGTCTTAGTTGCC
TCTAGAGAAATGGGTACCGCTTTGCAATTGGTTAACATTGCTAGAGAT
ATCAAGGGTGATGCCACTGAAGGTAGATTCTACTTGCCATTGTCCTTT
TTTGGTTTGAGAGACGAATCTAAGTTAGCTATTCCAACTGATTGGACC
GAACCAAGACCACAAGATTTTGACAAGTTGTTGTCTTTGTCCCCATCT
TCTACTTTACCATCTTCTAACGCTTCTGAATCTTTCAGATTTGAATGG
AAGACTTACTCCTTGCCATTGGTTGCTTATGCTGAAGATTTGGCTAAA
CACTCCTACAAGGGTATTGATAGATTGCCAACCGAGGTTCAAGCCGGT
ATGAGAGCTGCCTGTGCTTCCTACTTGTTGATCGGTAGAGAAATTAAG
GTTGTTTGGAAGGGTGACGTCGGTGAAAGAAGAACTGTTGCTGGTTGG
AGACGTGTCCGTAAAGTTTTGTCTGTTGTCATGTCTGGTTGGGAAGGT
CAATAGTTTTAGCCTCACCGATATACTTATATAATATTCATTGAAATA
GAAAGACATTTACAAAGTAGACGGGGATATTTAGCCCCACTCATATAA
ATTATGCATTATAACTTTTATAAAAAAAAAGAAATCACACTTAAATAA
ATGTCTTAATTGCACATTCCATGTTCAGATATACACTATCTTACCTTT
TGAATAGGAATGATGGAAAAAAAATTATGCTTCCAATCGGATTTGAAC
CGATGATCTCCACATTACTAGTGTGGCGCCTTACCAACTTGGCCATAG
AAGCCCGTTATTACGGTCTGTGATCCAGCAAATGTCAGATATAACGAA
TTGAAGTTGCTTTTCCCATCAATGTTTGTCAAGTTGAAACGCTGTAGC
ACACACTAAGCATTAACGACTATATTACAAGTAAAGCATCAAATGATT
ACTTAAAAGAGTAATCGCAAACTATATTGAAGTTGGCAAAGGCACATG
ATCCAAATGGTAACGATAAC.

The protein sequence of Xanthophyllomyces dendrorhous CrtYB is (SEQ ID NO:8):

MTALAYYQIHLIYTLPILGLLGLLTSPILTKFDIYKISILVFIAFSAT
TPWDSWIIRNGAWTYPSAESGQGVFGTFLDVPYEEYAFFVIQTVITGL
VYVLATRHLLPSLALPKTRSSALSLALKALIPLPIIYLFTAHPSPSPD
PLVTDHYFYMRALSLLITPPTMLLAALSGEYAFDWKSGRAKSTIAAIM
IPTVYLIWVDYVAVGQDSWSINDEKIVGWRLGGVLPIEEAMFFLLTNL
MIVLGLSACDHTQALYLLHGRTIYGNKKMPSSFPLITPPVLSLFFSSR
PYSSQPKRDLELAVKLLEEKSRSFFVASAGFPSEVRERLVGLYAFCRV
TDDLIDSPEVSSNPHATIDMVSDFLTLLFGPPLHPSQPDKILSSPLLP
PSHPSRPTGMYPLPPPPSLSPAELVQFLTERVPVQYHFAFRLLAKLQG
LIPRYPLDELLRGYTTDLIFPLSTEAVQARKTPIETTADLLDYGLCVA
GSVAELLVYVSWASAPSQVPATIEEREAVLVASREMGTALQLVNIARD
IKGDATEGRFYLPLSFFGLRDESKLAIPTDWTEPRPQDFDKLLSLSPS
STLPSSNASESFRFEWKTYSLPLVAYAEDLAKHSYKGIDRLPTEVQAG
MRAACASYLLIGREIKVVWKGDVGERRTVAGWRRVRKVLSVVMSGWEG
Q.

The Xanthophyllomyces dendrorhous CrtI gene was expressed from 0.61×pGAL1. The terminator on CrtI was tQCR10. CrtI encodes phytoene desaturase. (uniprot.org/uniprotkb/013506/entry). The DNA sequence of yeast codon-optimized XdCrtI, including two QCR10 terminators (SEQ ID NO:9):

ATGGGTAAAGAACAAGACCAAGATAAGCCAACTGCTATCATTGTTGGT
TGTGGTATTGGTGGTATCGCTACCGCCGCTAGATTGGCTAAAGAAGGT
TTCCAAGTTACTGTCTTCGAAAAGAACGATTATTCTGGTGGTAGATGC
TCTTTGATTGAAAGAGATGGTTATCGTTTCGACCAAGGTCCATCTTTG
TTGTTGTTGCCAGACTTGTTCAAGCAAACCTTCGAAGATTTGGGTGAA
AAGATGGAAGATTGGGTCGATTTGATCAAGTGTGAACCAAACTACGTT
TGTCATTTTCATGATGAAGAAACCTTCACTTTCTCTACCGACATGGCT
TTATTGAAGAGAGAAGTCGAAAGATTCGAAGGTAAGGATGGTTTCGAT
AGATTTTTATCTTTCATTCAAGAAGCTCATAGACACTACGAGTTGGCC
GTTGTTCATGTCTTACAAAAGAACTTCCCAGGTTTCGCTGCTTTCTTG
AGATTGCAATTTATCGGTCAAATTTTGGCTTTACATCCATTTGAATCT
ATCTGGACTAGAGTTTGTAGATACTTCAAGACCGATAGATTGAGAAGA
GTTTTTTCCTTCGCTGTTATGTACATGGGTCAATCTCCATACTCTGCC
CCTGGTACCTACTCCTTGTTACAATATACTGAATTGACTGAAGGTATC
TGGTATCCACGTGGTGGTTTTTGGCAAGTCCCAAACACCTTATTACAA
ATCGTCAAAAGAAACAACCCATCTGCTAAGTTCAACTTCAACGCTCCA
GTTTCTCAAGTCTTGTTGTCCCCAGCTAAGGATAGAGCTACTGGTGTT
AGATTGGAATCCGGTGAGGAACATCATGCCGACGTTGTCATCGTCAAC
GCTGACTTGGTCTACGCTTCTGAACACTTAATTCCAGATGACGCCAGA
AATAAAATCGGTCAATTGGGTGAAGTCAAGAGATCTTGGTGGGCCGAT
TTGGTCGGTGGTAAGAAGTTAAAAGGTTCTTGTTCCTCTTTATCCTTT
TACTGGTCTATGGATAGAATTGTCGATGGTTTGGGTGGTCATAACATT
TTCTTGGCTGAAGACTTCAAGGGTTCTTTTGACACCATTTTCGAGGAA
TTAGGTTTGCCAGCTGACCCATCTTTCTACGTTAACGTCCCATCCAGA
ATTGACCCATCTGCTGCTCCTGAAGGTAAAGACGCTATCGTTATTTTA
GTCCCATGTGGTCACATTGACGCCTCTAACCCACAAGACTACAATAAG
TTAGTCGCTAGAGCCAGAAAATTCGTTATTCAAACCTTGTCCGCTAAA
TTGGGTTTGCCAGACTTCGAAAAGATGATTGTTGCCGAAAAGGTTCAC
GACGCTCCATCCTGGGAAAAGGAATTCAATTTGAAAGATGGTTCCATC
TTGGGTTTGGCCCACAACTTCATGCAAGTCTTGGGTTTCAGACCATCT
ACCAGACACCCAAAATACGACAAGTTGTTTTTTGTCGGTGCCTCTACT
CACCCAGGTACCGGTGTTCCTATCGTTTTGGCTGGTGCCAAGTTAACT
GCTAATCAAGTTTTGGAATCTTTCGATCGTTCCCCAGCCCCAGATCCA
AACATGTCTTTGTCTGTTCCATATGGTAAGCCATTGAAGTCTAACGGT
ACCGGTATTGACTCCCAAGTCCAATTGAAGTTTATGGACTTGGAACGT
TGGGTTTACTTATTGGTCTTATTGATTGGTGCTGTCATTGCTAGATCC
GTCGGTGTCTTGGCTTTCTGAAGCCAAAACATTCAACCGGCTGAGTGA
TTTTATTTATTTGTTTTACGTTCTTTTCTATCATTTTGGAAGAGGAAT
TAATGCAAAACACATTTGTATTTTTTTTTATTTTTGCATATTCTCGAT
TATCTATACATATTTTTAAACTGGATCATAAAAGGTATAAAAAGTAAT
TGATCCATACAACATTTTTAAATATTTACCCCTCTATTTTCAGAATAT
TTACTAAAATATCAGTTATCAAAGAGTAGGGAAAAGTTAACTACAAGT
TTGTTTTTCCTCTTTTTATGTATCTTTTGAGCTTTGTAATTAGGCAGT
ACATCTCTATGTTTTCAGGGAAACCTATTTCATTCGCCATTTTGATGA
TTTAGGCATACGCCAATGCTAACGCCGTTTCTTGTAATTTCTATTTTT
TCAAATGGAGTACTTTACCTTCCTCCCGCAGCTGAAACTTTCAACCCG
CCGGAAGAAAAGACCAAATAATATTATAATTAATATTTAAAACC.

The protein sequence of Xanthophyllomyces dendrorhous CrtI is (SEQ ID NO:10):

MGKEQDQDKPTAIIVGCGIGGIATAARLAKEGFQVTVFEKNDYSGGRC
SLIERDGYRFDQGPSLLLLPDLFKQTFEDLGEKMEDWVDLIKCEPNYV
CHFHDEETFTFSTDMALLKREVERFEGKDGFDRFLSFIQEAHRHYELA
VVHVLQKNFPGFAAFLRLQFIGQILALHPFESIWTRVCRYFKTDRLRR
VFSFAVMYMGQSPYSAPGTYSLLQYTELTEGIWYPRGGFWQVPNTLLQ
IVKRNNPSAKFNFNAPVSQVLLSPAKDRATGVRLESGEEHHADVVIVN
ADLVYASEHLIPDDARNKIGQLGEVKRSWWADLVGGKKLKGSCSSLSF
YWSMDRIVDGLGGHNIFLAEDFKGSFDTIFEELGLPADPSFYVNVPSR
IDPSAAPEGKDAIVILVPCGHIDASNPQDYNKLVARARKFVIQTLSAK
LGLPDFEKMIVAEKVHDAPSWEKEFNLKDGSILGLAHNFMQVLGFRPS
TRHPKYDKLFFVGASTHPGTGVPIVLAGAKLTANQVLESFDRSPAPDP
NMSLSVPYGKPLKSNGTGIDSQVQLKFMDLERWVYLLVLLIGAVIARS
VGVLAF.

### 8.2.3 Production of β-Carotene by Y47247

Following growth in 96-well plates, Y47247 produced 33.4 g β-carotene. Production of β-carotene by Y47247 was considerably lower in bioreactors than it was in 96-well plates, possibly a reflection of batch compared to fed-batch growth conditions. Y47247 produced an average of 2.24 g/kg DCW of β-carotene with a peak titer of 2.5 g/kg DCW at Day 7 in fed-batch fermentation.

### 8.2.4 Production of β-Carotene by Y47245

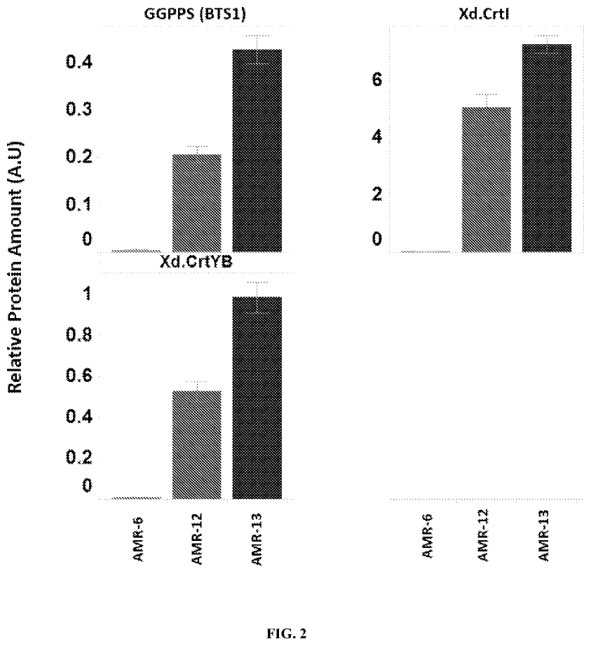

Another strain, Y47245, produced less β-carotene than Y47245. Protein expression levels of BTS1, CrtYB (=bifunctional phytoene synthase and lycopene cyclase) and CrtI (lycopene cyclase) were compared by mass spec. peptide abundance (as described above). The results of the mass spec. analysis are shown in FIG. 2, which demonstrates that both strains express these three proteins (or overexpress over the WT level in the case of BTS1), and that Y47247 (=AMR-13) expresses a higher concentration of these proteins than Y47245 (=AMR-12). The base strain, AMR-6 is Y35965 in Table 6-1.

### 8.3 Converting a β-Carotene Co-Producer Production Strain (AMR-13 Y47247) to a Canthaxanthin Co-Producer Production Strain

Canthaxanthin is a ketocarotenoid and an early oxidation product of β-carotene. Strain AMR-13 Y47247 was selected to explore production of canthaxanthin because it had a higher accumulation of β-carotene per unit of biomass than strain AMR-12 Y47245. The minimal (<10%) reductions in farnesene yield and volumetric productivity were deemed acceptable with the hope of capturing the higher carotenoid titers.

| | | Change in | | |
|---|---|---|---|---|
| | Change in | relative | | |
| | relative | farnesene | | |
| | farnesene | volumetric | β-carotene | Canthaxanthin |
| Strain | Yield | productivity | (g/kg DCW) | (g/kg DCW) |
| AMR-12 | ND | ND | 1.47 ± 0.24 | NA |
| (Y47245) | | | | |
| AMR-13 | 5.7% lower | 6.5% lower | 2.24 ± 0.20 | NA |
| (Y47247) | | | | |
| AMR-28 | ND | 8% lower | 0.57 ± 0.09 | 2.07 ± 0.15 |

Table 6-2 provides performance of strains AMR-12, AMR-13, and AMR-28 in 0.5-L bioreactors in an 8-day, fed batch fermentation (N=2 for AMR-12 and AMR-13; N=1 for AMR-28). Percent changes in farnesene yield and productivity for strains AMR-12, AMR-13, and AMR-28 relative to parental farnesene manufacturing strain, AMR-6, are shown. The β-carotene and canthaxanthin titers represent the average of values of each sample taken from Day 4 through Day 8 of the fermentation runs. Duplicate samples were taken for each tank at each time point. Changes in relative farnesene yield and volumetric productivity for strain AMR-12 and volumetric productivity for AMR-28 were not detected (ND). NA: Not assayed.

8.3.1 Titrating Expression of Paracoccus sp. CrtW (Encoding β-Carotene Ketolase)

A range of promoter strengths (Table 6-1 and Table 6-3) were used to express the Paracococus sp. β-carotene ketolase (CrtW), an enzyme that converts β-carotene to canthaxanthin, in strain AMR-13, generating strains AMR-16 to AMR-26 (Table 6-1).

The Paracococus sp. β-carotene ketolase (encoded by CrtW): encodes an enzyme with carotenoid-4,4′-b-ionone ring oxygenase activity. In Table 6-1, this gene is referred to as Pa. CrtW. The yeast optimized sequence used herein (SEO ID NO: 3):

ATGTCAGCACACGCACTTCCTAAAGCAGATTTGACTGCTACTTCTTTG
ATCGTTTCTGGTGGTATTATTGCTGCCTGGTTAGCCTTGCACGTTCAC
GCTTTGTGGTTCTTAGACGCTGCTGCTCATCCAATTTTAGCTATTGCT
AATTTTTTGGGTTTAACTTGGTTGTCCGTTGGTTTGTTCATTATTGCC
CACGACGCTATGCACGGTTCTGTCGTCCCTGGTAGACCAAGAGCCAAC
GCTGCCATGGGTCAATTAGTTTTGTGGTTGTACGCCGGTTTCTCCTGG
CGTAAGATGATCGTTAAGCATATGGCTCACCATCGTCACGCTGGTACC
GACGACGATCCTGACTTCGACCACGGTGGTCCAGTTAGATGGTACGCC
AGATTTATCGGTACTTACTTCGGTTGGAGAGAAGGTTTGTTGTTGCCA
GTTATCGTCACCGTTTACGCTTTAATCTTGGGTGATAGATGGATGTAC
GTTGTCTTCTGGCCATTGCCATCTATTTTGGCCTCTATTCAATTATTC
GTCTTTGGTACCTGGTTGCCACACAGACCAGGTCACGACGCTTTCCCA
GACAGACACAACGCCCGTTCTTCTAGAATCTCTGATCCAGTCTCTTTA
TTGACCTGTTTCCATTTCGGTGGTTACCATCATGAACATCACTTACAC
CCAACTGTCCCATGGTGGAGATTGCCATCCACTAGAACTAAAGGTGAC
ACTGCTTAAATTGAATTGAATTGAAATCGATAGATCAATTTTTTTCTT
TTCTCTTTCCCCATCCTTTACGCTAAAATAATAGTTTATTTTATTTTT
TGAATATTTTTTATTTATATACGTATATATAGACTATTATTTATCTTT
TAATGATTATTAAGATTTTTATTAAAAAAAAATTCGCTCCTCTTTTAA
TGCCTTTATGCAGTTTTTTTTTCCCATTCGATATTTCTATGTTCGGGT
TCAGCGTATTTTAAGTTTA

The protein sequence of Paracoccus β-carotene ketolase is (SEQ ID NO:4):

MSAHALPKADLTATSLIVSGGIIAAWLALHVHALWFLDAAAHPILAIA
NFLGLTWLSVGLFIIAHDAMHGSVVPGRPRANAAMGQLVLWLYAGFSW
RKMIVKHMAHHRHAGTDDDPDFDHGGPVRWYARFIGTYFGWREGLLLP
VIVTVYALILGDRWMYVVFWPLPSILASIQLFVFGTWLPHRPGHDAFP
DRHNARSSRISDPVSLLTCFHFGGYHHEHHLHPTVPWWRLPSTRTKGD
TA.

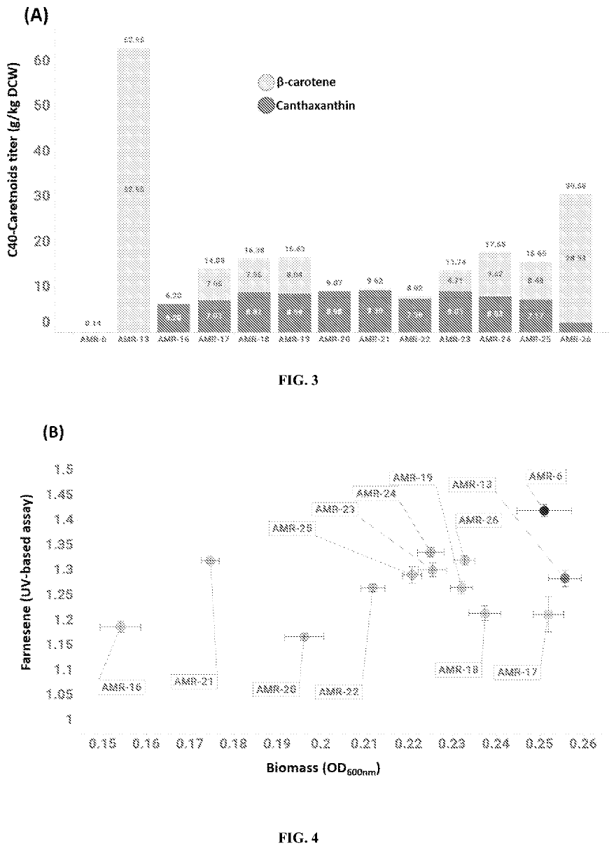

The strain with the lowest level of CrtW expression (AMR-26) gave the lowest canthaxanthin titer (FIG. 3). The strain with the highest level of CrtW expression (strain AMR-16) has the second lowest canthaxanthin titer of the set. Further, AMR-16 produced less biomass and farnesene (FIG. 4B), suggesting that strongly overexpressing this integral membrane protein has a detrimental effect on strain physiology. Strains AMR-18 to AMR-21 had similar levels of CrtW expression (Table 6-3) and similar levels of canthaxanthin production, making them equally desirable despite difference in β-carotene levels. Strain AMR-23 (=Y51691) was selected since it had the lowest expression of CrtW, based on the predicted strength of the promoter, and because farnesene production was unaffected compared to AMR-13 (FIG. 3, FIG. 4).

| | Paracococus sp. | | | | |
|---|---|---|---|---|---|
| | CrtW | | | | |
| | Promoter | | | | |
| | Strength | | | | |
| | relative | Canthaxanthin | β-carotene | Farnesene | Biomass |
| Strain | to pGAL1 | (g/kg DCW) | (g/kg DCW) | (UV assay) | (OD600) |
| AMR- | n/a | 0 | 0.14 | 1.42 | 0.25 |
| 6 | | | | | |
| AMR- | n/a | 0 | 62.95 | 1.28 | 0.26 |
| 13 | | | | | |
| AMR- | 0.54 | 6.20 | 0 | 1.19 | 0.15 |
| 16 | | | | | |
| AMR- | 0.37 | 7.03 | 7.05 | 1.21 | 0.25 |
| 17 | | | | | |
| AMR- | 0.36 | 8.82 | 7.55 | 1.21 | 0.24 |
| 18 | | | | | |
| AMR- | 0.36 | 8.59 | 8.04 | 1.27 | 0.23 |
| 19 | | | | | |
| AMR- | 0.36 | 8.98 | 0.09 | 1.17 | 0.20 |
| 20 | | | | | |
| AMR- | 0.35 | 9.30 | 0.32 | 1.32 | 0.17 |
| 21 | | | | | |
| AMR- | 0.32 | 7.39 | 0.63 | 1.26 | 0.21 |
| 22 | | | | | |
| AMR- | 0.25 | 9.03 | 4.71 | 1.30 | 0.23 |
| 23 | | | | | |
| AMR- | 0.21 | 8.03 | 9.62 | 1.34 | 0.23 |
| 24 | | | | | |
| AMR- | 0.11 | 7.17 | 8.48 | 1.29 | 0.22 |
| 25 | | | | | |
| AMR- | 0.05 | 2.05 | 28.53 | 1.32 | 0.23 |
| 26 | | | | | |

Table 6-3: Expression of the Paracococus sp. CrtW genes using a range of promoter strengths in the β-carotene co-producer strain AMR-13. Promoter strengths are relative to pGAL1.

### 8.3.2 AMR-23 Produced Echinenone, an Intermediate in the Conversion of β-Carotene to Canthaxanthin

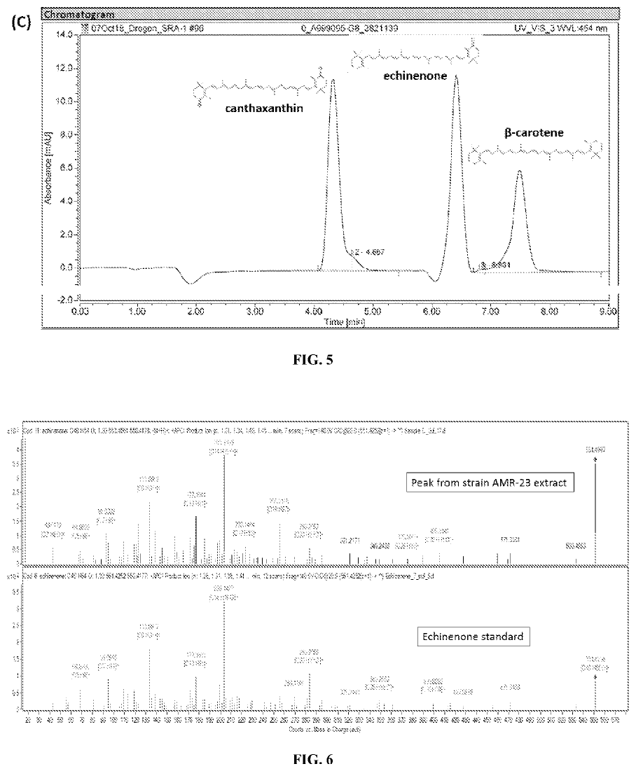

In all tested cases, expression of the CrtW gene resulted in an apparent loss of some C40 product (FIG. 3): the sum of canthaxanthin and remaining β-carotene was lower than the β-carotene produced by the parent strain, AMR-13 (63 g/kg DCW). AMR-23, for example, had one of the highest canthaxanthin titers of 9.03 g/kg DCW but only 4.71 g/kg DCW of remaining β-carotene (FIG. 3). However, chromatography revealed that strain AMR-23 made a significant amount of an intermediate molecule that appears to be more polar than β-carotene but less polar than canthaxanthin (FIG. 5). Retention time match by UV-UPLC analysis and mass spectrometric analysis (FIG. 6) both support assignment of this molecule as echinenone, a mono-ketolated β-carotene.

Subsequent strain engineering measured success in converting echinenone to canthaxanthin by both the relative reduction in echinenone chromatographic peak height and the increase in canthaxanthin absolute quantity.

That the majority of β-carotene is converted to echinenone and only a fraction of the echinenone is converted to canthaxanthin indicates that the first ketolation (of a less polar β-carotene) happens more readily than the second ketolation (of a more polar echinenone). Possible explanations for this observation include a differential specificity of the CrtW from Paracococus sp. for β-carotene and echinenone, or that the availability of the substrates to the enzyme is different.

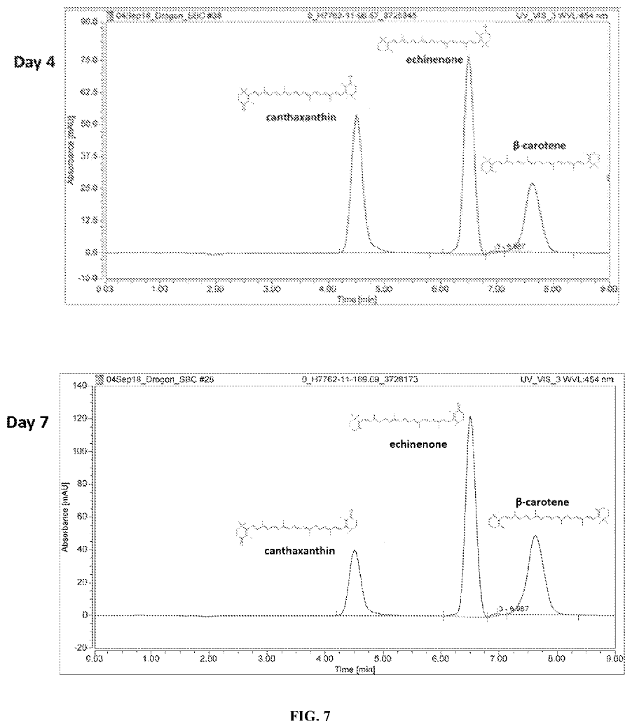

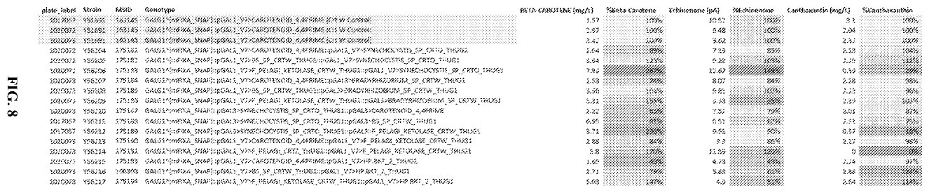

Strain AMR-23 was scaled up to 0.5 L fed-batch fermentation tanks, showing a visibly redder color than AMR-13. It produced an average of 1 g/kg DCW of canthaxanthin in 8-day fed batch fermentation (N=2). At every timepoint the strain accumulated a significant amount of echinenone that, if chromatographic response factors are similar, matched or exceeded the production of canthaxanthin (FIG. 7). The chromatographic analysis shows that the peak height ratio of echinenone to canthaxanthin increased from ~1.2:1 at Day 4 to ~3:1 on Day 7, indicating that second ketolation became less efficient over the course of the fed-batch fermentation.

### 8.3.3 Improving Conversion of β-Carotene to Canthaxanthin

To resolve the inability of Paracoccus sp. CrtW to efficiently complete the conversion of β-carotene to canthaxanthin, enzyme biodiversity was screened for an enzyme that either could complete the reaction on its own or could effectively convert echinenone to canthaxanthin. Since canthaxanthin is a naturally occurring pigment present in bacteria, algae and some fungi, there is a pool of diverse enzymes in nature that can be identified bioinformatically.

A broad pool of CrtW enzyme variants available for our screen was narrowed using the following criteria, listed in descending order of weighting: (i) PubMed abstracts mentioning CrtW and that also mention canthaxanthin or a specific host organism, (ii) existence of km values in BRENDA (Jeske et al., 2019, Nucleic Acids Res. 8 (47): D542-D549, (iii) experimental evidence for the existence of the genetic sequence at the protein or mRNA level from the Uniprot database, (iv) phylogenetic tree topology, and (v) sequence diversity from a multiple sequence alignment. Based on enzyme selection findings, twenty-four different new genes were codon optimized and synthesized (Table 6-4).

TABLE 6-4
Species and accession numbers for the
screen of β-carotene ketolase (CrtW).

| Species | Accession Number |
|---|---|
| Rhizobium sp. Leaf321 | WP_062583351 |
| Xanthobacter sp. | A0A259BJG0 |
| Parvularcula oceani | WP_031553298 |
| Erythrobacter luteus | A0A0G9MT70 |
| Fulvimarina pelagi | Q0G1H1 |
| Sphingomonas melonis | A3QQZ2 |
| Sphingomonas guangdongensis | A0A285R712 |
| Phenylobacterium deserti | A0A328ACX2 |
| Brevundimonas sp. | Q4W8B8 |
| Brevundimonas vesicularis | Q0H2C9 |
| Hyphomicrobium sp. | PPD30519 |
| Bradyrhizobium sp. ORS 278 | Q9KIX0 |
| Paracoccus sp. | P54972 |
| Paracoccus haeundaensis | Q24K64 |
| Nostoc sp. PCC 7120 | Q8YSA0 |
| Haematococcus pluvaialis | Q39982 |
| Agrobacterium radiobacter K84 | B9JL03 |
| Synechocystis sp. PCC6803 | Q55808 |
| Nocardia farcinica IFM 10152 | Q5YYZ2 |
| Mycobacterium smegmatis str. MC2 155 | A0QTL4 |
| Arthrospira platensis NIES-39 | D4ZU91 |
| Flavobacteria bacterium MS024-3C | C0BNG6 |
| Caenorhabditis briggsae | A8WQT8 |
| Fulvimarina pelagi | Q0G1G7 |
| Prochlorococcus marinus | Q7V092 |
| subsp. pastoris str. CCMP1986 | |
| Myxococcus xanthus DK 1622 | Q1D6N4 |
| Myxococcus xanthus DK 1622 | Q1D169 |
| Bacillus clausii KSM-K16 | Q5WK32 |

### 8.3.4 Screening Selected Putative β-Carotene Ketolases for Conversion of β-Carotene to Canthaxanthin

Selected CrtW genes were integrated into the genome of Y47247 (=AMR-13, the β-carotene producing strain we chose to use) and the genes expressed. Production of carotenoids and ketocarotenoids was assayed following growth in 96-well plates as described above.

FIG. 8 shows carotenoid and ketocarotenoid production from strains engineered to express various combinations of CrtW genes. Below are descriptions of the β-carotene ketolase genes expressed in each strain (they're not all called CrtW in Genbank or Uniprot):

### 8.3.4.1 Y56217

Gene 1: Genotype description in FIG. 8 is HP_BKT2. Haematococcus lacustris β-carotene ketolase. UniProtKB/Swiss-Prot: Q39982. (ncbi.nlm.nih.gov/protein/Q39982.1?report-genbank&log$=protalign&blast_rank=2&RID=P0 SWDU65013). Yeast codon-optimized DNA sequence (SEQ ID NO:11)

ATGCACGTAGCCAGTGCTTTAATGGTAGAACAAAAGGGTTCTGAAGCC
GCTGCTTCTTCTCCAGATGTTTTGAGAGCTTGGGCCACTCAATACCAC
ATGCCATCCGAATCCTCTGACGCTGCCAGACCAGCTTTGAAGCACGCT
TACAAGCCACCAGCCTCTGACGCTAAGGGTATTACTATGGCCTTAACT
ATTATTGGTACCTGGACTGCTGTTTTCTTGCACGCCATCTTTCAAATT
AGATTGCCAACTTCCATGGACCAATTGCACTGGTTGCCAGTTTCCGAA
GCCACCGCTCAATTGTTAGGTGGTTCTTCCTCCTTGTTACATATTGCC
GCTGTCTTCATTGTTTTGGAATTTTTATACACCGGTTTGTTCATTACC
ACCCATGACGCTATGCATGGTACTATCGCCTTGAGACACAGACAATTA
AATGACTTATTGGGTAACATCTGTATCTCTTTGTACGCTTGGTTCGAC
TACTCTATGTTGCATAGAAAGCACTGGGAACACCACAACCACACTGGT
GAAGTTGGTAAAGACCCAGACTTTCATAAGGGTAACCCTGGTTTGGTT
CCATGGTTTGCTTCCTTCATGTCTTCTTACATGTCCTTGTGGCAATTC
GCCCGTTTGGCTTGGTGGGCCGTTGTTATGCAAATGTTAGGTGCCCCA
ATGGCTAACTTGTTGGTTTTCATGGCTGCTGCTCCAATCTTGTCTGCT
TTCAGATTGTTCTACTTTGGTACCTACTTGCCTCACAAGCCAGAACCA
GGTCCAGCTGCTGGTTCCCAAGTTATGGCTTGGTTCAGAGCTAAGACT
TCCGAGGCTTCTGACGTTATGTCCTTTTTGACCTGTTACCATTTTGAT
TTACACTGGGAACACCACAGATGGCCATTCGCCCCATGGTGGCAATTA
CCTCACTGTAGAAGATTGTCCGGTAGAGGTTTGGTTCCAGCTTTGGCC
TAAGAGTATGCTTCTCTTTTTTTTTGTAGGCCAGTGATAGGAAAGAAC
AATAGAATATAAATACGTCAGAATATAATAGATATGTTTTTATATTTA
GACCTCGTACATAGGAATAATTGACGTTTTTTTTGGCCAACATTTGAA
ATTTTTTTTTGTTACCTCGCGCTGAGCCCAAACGGGCTCCACTACCCG

Gene 2: Genotype description in FIG. 8 is F_pelagi_ketolase_CrtW, a fatty acid desaturase [Fulvimarina pelagi]. NCBI Reference Sequence: WP_081448320.1 (ncbi.nlm.nih.gov/protein/WP_081448320.1?report-genbank&log$-protalign&blast_rank=1& RID=P0V2C0HC01R). Yeast codon-optimized DNA sequence: (SEQ ID NO:12):

ATGACTTTATCTCCTACTTCTCGTTTAATCCCAGCCTCCGCCTTGCCA
CGTTCCACTCCAGCCGACTCTCCAAAGATCAGACCATACCAAACTACC
ATCGGTTTGACTTTGTGTGCTGTTTTGTTGGCTTCTTGGTTTGCTATT
CACGTTTCCGCTATCTTCTTCTTAGATATCAACTTCTCTACTTTGCCA
TTAGCCCCATTGATTACCGTCTTTCAATGTTGGTTGACTGTCGGTTTA
TTTATTTTGGCTCATGACGCCATGCATGGTTCTTTGGCTCCAGGTAGA
ACTCGTTTGAACGCTGTTATTGGTGGTTTCATTTTGTTTGTTTATGCC
GGTTTCGCTTGGAAAAAGATTAGAGATGCCCACTTCGCTCACCACGAT
GCTCCTGGTACCCCTGCTGATCCAGACTTCTACGCTGATGACCCAGAA
AATTTCTGGCCATGGTTCGGTACCTTCTTTTCTAGATACTTCGGTTGG
AGATCTGTCGCCTTCGTTTCTACTGTTGTCACCTTCTACTTAGTTATT
TTGGATGCCTCTGTCACTAACGTCGTCTTGTTCTATGGTTTGCCTTCC
TTGTTGTCTTCCTTGCAATTGTTTTACTTCGGTACTTACAGACCACAC
AGACACGAAGAATCTGGTACTTTCGCCGATGCCCACAACACTCGTTCT
TCTGAATTCGGTTATGTTGCTTCCTTGTTCTCCTGCTTCCACTTCGGT
TACCACCACGAACATCACTTGGCCCCATGGACTCCTTGGTGGGCTTTA
CCTCATACTAGACAATCTTAAGAGTATGCTTCTCTTTTTTTTTGTAGG
CCAGTGATAGGAAAGAACAATAGAATATAAATACGTCAGAATATAATA
GATATGTTTTTATATTTAGACCTCGTACATAGGAATAATTGACGTTTT
TTTTGGCCAACATTTGAAATTTTTTTTTGTTACCTCGCGCTGAGCCCA
AACGGGCTCCACTACCCGCCGCGGTCGCC

### 8.3.4.2 Y56216

Gene 1: Genotype description in FIG. 8 is BS_SP_CRTW. Several differently named genes match this sequence in BLAST_X. Closest (100% match) are GenBank: BDC30290.1 (ncbi.nlm.nih.gov/protein/BDC30290.1?report-genbank&log$-protalign&blast_rank=1&RID=P0XP61Z7013) and GenBank: AHM24029.1 (ncbi.nlm.nih.gov/protein/AHM24029.1?report-genbank&log$=protalign&blast_rank=2&RID=P0XP61Z7013). The gene apparently comes from Brevundimonas SD212 (MBIC 03018). Yeast codon-optimized DNA sequence (SEQ ID NO:13):

ATGACAGCAGCCGTTGCTGAACCTAGAATCGTCCCAAGACAAACTTGG
ATCGGTTTGACCTTGGCCGGTATGATCGTTGCCGGTTGGGGTTCCTTG
CACGTTTACGGTGTTTACTTTCATCGTTGGGGTACCTCTTCCTTAGTC
ATTGTTCCAGCTATTGTCGCTGTTCAAACTTGGTTGTCCGTTGGTTTG
TTCATTGTTGCTCACGATGCTATGCACGGTTCTTTGGCCCCAGGTAGA
CCAAGATTGAACGCTGCTGTTGGTCGTTTGACCTTGGGTTTGTATGCC
GGTTTTAGATTTGACAGATTGAAGACCGCTCACCACGCCCACCACGCT
GCCCCAGGTACTGCCGATGACCCTGACTTCTACGCTCCAGCTCCAAGA
GCTTTCTTGCCATGGTTTTTGAACTTCTTTAGAACCTATTTCGGTTGG
AGAGAAATGGCTGTCTTGACTGCCTTGGTTTTAATCGCTTTATTCGGT
TTGGGTGCTAGACCAGCTAACTTATTAACCTTCTGGGCCGCTCCAGCT
TTGTTGTCTGCCTTGCAATTATTCACTTTCGGTACTTGGTTGCCTCAC
AGACACACTGACCAACCATTCGCTGACGCTCACCATGCCAGATCTTCT
GGTTACGGTCCAGTTTTGTCTTTGTTAACCTGTTTTCACTTCGGTCGT
CACCATGAACACCATTTAACTCCATGGCGTCCATGGTGGAGATTGTGG
AGAGGTGAATCTTAAGAGTATGCTTCTCTTTTTTTTTGTAGGCCAGTG
ATAGGAAAGAACAATAGAATATAAATACGTCAGAATATAATAGATATG
TTTTTATATTTAGACCTCGTACATAGGAATAATTGACGTTTTTTTTGG
CCAACATTTGAAATTTTTTTTTGTTACCTCGCGCTGAGCCCAAACGGG
CTCCACTACCCGCCGCGGTCGCC

Gene 2: Genotype description in FIG. 8 is HP_BKT2. See Y56217.

### 8.3.4.3 Y56215

Gene 1: FIG. 8 description is CAROTENOID_4,4PRIME: It encodes Paracococus sp. β-carotene ketolase (encoded by CrtW): Enzyme activity: carotenoid-4,4′-b-ionone ring oxygenase. See above for AMR-16 to AMR-26 (Table 6-3).

Gene 2: Genotype description in FIG. 8 is HP_BKT2. See Y56217.

### 8.3.4.4 Y56214

Gene 1: F_PELAGI_CRTZ. sterol desaturase family protein (Fulvimarina pelagi). (ncbi.nlm.nih.gov/protein/WP_007067657.1?report=genbank&log$=protalign&blast_rank=1& RID=P34YU8SA013). Note that there are five identical proteins in Genbank, some which are annotated as ‘Carotene hydroxylase’ (ncbi.nlm.nih.gov/ipg/WP_007067657.1). Yeast codon-optimized DNA sequence (SEQ ID NO:14)

ATGACTATATGGACTCTTTACTATGTATGTTTGACCTTGGTCACTATC
GGTTTGATGGAAGTTTACGCTTGGTGGGCTCATAAGTTCATTATGCAC
GGTAAATTCGGTTGGGGTTGGCACAAATCCCATCACGAAGAAACTGAA
GGTTGGTTTGAAAAGAATGACTTATACGCTGTTGTTTTCGCTGGTTTT
GCTATCGCCTTGTTCATGGTTGGTCACTTCTTGTCCCCAACTTTGTTG
GCCATTGCTTGGGGTATCACTTTATACGGTTTGTTATACTTCGTTGCT
CATGACGGTTTGGTCCATCAAAGATGGCCTTTTAACTACGTTCCACAC
AGAGGTTATGCTAAGCGTTTAGTTCAAGCTCATAGATTACACCACGCT
GTTGAAGGTAGAGAACACTGTGTCTCCTTCGGTTTCTTATACGCTCCA
CCTATTGAAAAGTTGAAGCGTGATTTGCGTGAATCCGGTATTTTAGAA
CGTGAAAGAATCGAACGTTCTTTAGACCAACAAGGTTCTGCTCACGCC
CCAGTTAGATAAGAGTATGCTTCTCTTTTTTTTTGTAGGCCAGTGATA
GGAAAGAACAATAGAATATAAATACGTCAGAATATAATAGATATGTTT
TTATATTTAGACCTCGTACATAGGAATAATTGACGTTTTTTTTGGCCA
ACATTTGAAATTTTTTTTTGTTACCTCGCGCTGAGCCCAAACGGGCTC
CACTACCCGCCGCGGTCGCC

Gene 2: Genotype description in FIG. 8 is F_pelagi_ketolase_CrtW . . . . See Y56217, Gene 2.

### 8.3.4.5 Y56213

Gene 1: FIG. 8 description is CAROTENOID_4,4PRIME: Paracococus sp. β-carotene ketolase (encoded by CrtW). Enzyme activity: carotenoid-4,4′-b-ionone ring oxygenase. See above for AMR-16 to AMR-26 (Table 6-3).

Gene 2: Genotype description in FIG. 8 is F_pelagi_ketolase_CrtW. See Y56217.

### 8.3.4.6 Y56212

Gene 1: Genotype description in FIG. 8 is SYNECHOCYSTIS_SP_CRTO. MULTISPECIES: NAD (P)/FAD-dependent oxidoreductase (unclassified Synechocystis). NCBI Reference Sequence: WP_010873789.1 (ncbi.nlm.nih.gov/protein/WP_010873789.1?report=genbank&log$=protalign&blast_rank=1& RID=P373YUZ3013). Yeast codon-optimized DNA sequence (SEQ ID NO:15):

ATGATTACAACCGATGTAGTCATTATAGGTGCTGGTCATAACGGTTTG
GTCTGCGCTGCCTACTTATTACAACGTGGTTTGGGTGTCACTTTGTTA
GAAAAGAGAGAAGTTCCAGGTGGTGCTGCTACTACCGAAGCCTTAATG
CCTGAATTGTCCCCACAATTCAGATTTAACAGATGCGCCATCGACCAT
GAATTCATTTTCTTAGGTCCTGTCTTGCAAGAATTGAACTTAGCTCAA
TACGGTTTGGAATACTTGTTCTGTGATCCATCCGTTTTCTGTCCAGGT
TTAGATGGTCAAGCTTTCATGTCTTACCGTTCCTTAGAAAAGACCTGT
GCTCACATTGCTACTTATTCCCCAAGAGACGCTGAAAAGTACCGTCAA
TTTGTTAACTACTGGACTGACTTGTTAAATGCCGTTCAACCTGCTTTT
AACGCTCCACCACAAGCTTTGTTGGACTTGGCCTTGAACTACGGTTGG
GAGAACTTAAAGTCTGTCTTAGCCATCGCCGGTTCCAAGACCAAAGCT
TTGGACTTCATTAGAACTATGATTGGTTCCCCTGAAGACGTTTTGAAC
GAATGGTTCGACTCTGAAAGAGTCAAAGCTCCATTAGCTAGATTGTGT
TCCGAAATTGGTGCCCCTCCTTCTCAAAAGGGTTCTTCTTCTGGTATG
ATGATGGTCGCTATGAGACACTTAGAAGGTATTGCTCGTCCAAAAGGT
GGTACTGGTGCTTTGACCGAGGCCTTGGTCAAATTGGTTCAAGCTCAA
GGTGGTAAGATCTTGACTGATCAAACTGTTAAGCGTGTCTTGGTCGAA
AACAACCAAGCTATTGGTGTTGAAGTCGCCAACGGTGAACAATACAGA
GCTAAAAAAGGTGTCATTTCCAACATTGATGCCCGTCGTTTGTTTTTA
CAATTAGTCGAACCAGGTGCTTTAGCCAAAGTTAACCAAAACTTGGGT
GAACGTTTGGAAAGAAGAACCGTTAACAACAACGAAGCCATTTTGAAG
ATTGATTGTGCTTTGTCTGGTTTGCCTCACTTCACTGCTATGGCTGGT
CCAGAAGATTTGACTGGTACTATTTTGATTGCTGACTCCGTCCGTCAC
GTCGAAGAAGCCCATGCCTTAATTGCTTTGGGTCAAATTCCTGATGCT
AACCCATCTTTGTACTTAGATATTCCAACTGTTTTGGACCCTACTATG
GCTCCTCCAGGTCAACATACTTTGTGGATCGAGTTTTTCGCCCCTTAC
AGAATCGCTGGTTTGGAAGGTACCGGTTTAATGGGTACTGGTTGGACT
GATGAATTGAAGGAAAAGGTCGCTGATAGAGTTATCGATAAATTGACC
GACTACGCTCCAAACTTAAAGTCCTTGATCATCGGTAGAAGAGTCGAA
TCTCCAGCCGAATTAGCTCAACGTTTAGGTTCTTACAACGGTAATGTT
TACCACTTAGACATGTCTTTAGATCAAATGATGTTCTTGCGTCCATTA
CCAGAAATCGCCAACTACCAAACCCCTATCAAGAACTTGTACTTAACC
GGTGCTGGTACTCATCCAGGTGGTTCTATTTCTGGTATGCCTGGTAGA
AACTGTGCCAGAGTCTTCTTAAAGCAACAAAGACGTTTCTGGTAAGAG
TATGCTTCTCTTTTTTTTTGTAGGCCAGTGATAGGAAAGAACAATAGA
ATATAAATACGTCAGAATATAATAGATATGTTTTTATATTTAGACCTC
GTACATAGGAATAATTGACGTTTTTTTTGGCCAACATTTGAAATTTTT
TTTTGTTACCTCGCGCTGAGCCCAAACGGGCTCCACTACCCGCCGCGG
TCGCC

Gene 2: Genotype description in FIG. 8 is F_pelagi_ketolase_CrtW. See Y56217.

### 8.3.4.7 Y56211

Gene 1: Genotype description in FIG. 8 is SYNECHOCYSTIS_SP_CRTO. See Y56212, Gene 1.

Gene 2: Genotype description in FIG. 8 is BS_SP_CRTW. See Y56216, Gene 2.

### 8.3.4.8 Y56210

Gene 1: Genotype description in FIG. 8 is SYNECHOCYSTIS_SP_CRTO. See Y56212, Gene 1.

Gene 2: FIG. 8 description is CAROTENOID_4,4PRIME: It encodes Paracococus sp. β-carotene ketolase (encoded by CrtW). Enzyme activity: carotenoid-4,4′-b-ionone ring oxygenase. See above for AMR-16 to AMR-26 (Table 6-3).

### 8.3.4.9 Y56209

Gene 1: Genotype description in FIG. 8 is F_pelagi_ketolase_CrtW. See Y56217.

Gene 2: FIG. 8 description is BRADYRHIZOBIUM_SP_CRTW. Described as fatty acid desaturase (Bradyrhizobium sp. ORS 278): NCBI Reference Sequence: WP_012029995.1 (ncbi.nlm.nih.gov/protein/WP_012029995.1?report-genbank&log$=protalign&blast_rank=1& RID=P3917W68013). Yeast codon-optimized DNA sequence (SEQ ID NO:1).:

ATGCACGCAGCCACTGCCAAAGCCACAGAGTTTGGTGCCTCCCGTAGA
GACGATGCTAGACAAAGAAGAGTCGGTTTGACTTTAGCTGCCGTTATC
ATCGCTGCTTGGTTGGTCTTGCACGTCGGTTTGATGTTCTTCTGGCCA
TTGACTTTGCACTCTTTGTTACCAGCTTTACCATTGGTCGTCTTGCAA
ACTTGGTTGTACGTTGGTTTGTTCATTATTGCTCACGACTGTATGCAC
GGTTCTTTGGTTCCATTCAAACCTCAAGTCAACAGAAGAATCGGTCAA
TTGTGCTTGTTTTTGTACGCTGGTTTCTCTTTCGACGCTTTAAACGTT
GAACACCACAAACATCATCGTCATCCAGGTACTGCTGAAGATCCTGAT
TTTGACGAAGTCCCTCCACATGGTTTTTGGCATTGGTTCGCTTCCTTT
TTCTTGCACTACTTCGGTTGGAAACAAGTTGCCATTATCGCTGCCGTC
TCCTTAGTTTACCAATTAGTTTTTGCTGTCCCATTGCAAAACATCTTA
TTGTTCTGGGCTTTACCTGGTTTATTGTCCGCCTTGCAATTGTTCACT
TTCGGTACTTATTTACCACATAAGCCAGCTACCCAACCTTTTGCTGAC
AGACATAACGCCAGAACCTCTGAATTCCCAGCCTGGTTGTCTTTATTG
ACCTGTTTCCACTTCGGTTTCCACCACGAACACCATTTGCATCCAGAC
GCTCCATGGTGGCGTTTACCAGAAATCAAGAGACGTGCCTTGGAAAGA
AGAGACTAAGAGTATGCTTCTCTTTTTTTTTGTAGGCCAGTGATAGGA
AAGAACAATAGAATATAAATACGTCAGAATATAATAGATATGTTTTTA
TATTTAGACCTCGTACATAGGAATAATTGACGTTTTTTTTGGCCAACA
TTTGAAATTTTTTTTTGTTACCTCGCGCTGAGCCCAAACGGGCTCCAC
TACCCGCCGCGGTCGCC.

The protein sequence of Bradyrhizobium β-carotene ketolase is (SEQ ID NO:2):

MHAATAKATEFGASRRDDARQRRVGLTLAAVIIAAWLVLHVGLMFFWP
LTLHSLLPALPLVVLQTWLYVGLFIIAHDCMHGSLVPFKPQVNRRIGQ
LCLFLYAGFSFDALNVEHHKHHRHPGTAEDPDFDEVPPHGFWHWFASF
FLHYFGWKQVAIIAAVSLVYQLVFAVPLQNILLFWALPGLLSALQLFT
FGTYLPHKPATQPFADRHNARTSEFPAWLSLLTCFHFGFHHEHHLHPD
APWWRLPEIKRRALERRD.

### 8.3.4.10 Y56208

Gene 1: Genotype description in FIG. 8 is BS_SP_CRTW. See Y56216.

Gene 2: FIG. 8 description is BRADYRHIZOBIUM_SP_CRTW. See Y56209, Gene 2.

### 8.3.4.11 Y56207

Gene 1: FIG. 8 description is CAROTENOID_4,4PRIME: Paracococus sp. β-carotene ketolase (encoded by CrtW): Enzyme activity: carotenoid-4,4′-b-ionone ring oxygenase. See above for AMR-16 to AMR-26 (Table 6-3).

Gene 2: FIG. 8 description is BRADYRHIZOBIUM_SP_CRTW . . . . See Y56209, Gene 2.

### 8.3.4.12 Y56206

Gene 1: Genotype description in FIG. 8 is F_pelagi_ketolase_CrtW. Sec Y56217, Gene 2.

Gene 2: Genotype description in FIG. 8 is SYNECHOCYSTIS_SP_CRTO. See Y56212, Gene 1.

### 8.3.4.13 Y56205

Gene 1: Genotype description in FIG. 8 is BS_SP_CRTW. See Y56216, Gene 1.

Gene 2: Genotype description in FIG. 8 is SYNECHOCYSTIS_SP_CRTO. See Y56212, Gene 1.

### 8.3.4.14 Y56204

Gene 1: FIG. 8 description is CAROTENOID_4,4PRIME. Paracococus sp. β-carotene ketolase (encoded by CrtW): Enzyme activity: carotenoid-4,4′-b-ionone ring oxygenase. See above for AMR-16 to AMR-26 (Table 6-3).

Gene 2: Genotype description in FIG. 8 is SYNECHOCYSTIS_SP_CRTO. See Y56212, Gene 1.

8.3.5 the β-Carotene Ketolase from Bradyrhizobium sp. Strain ORS278 was the Most Active Enzyme for Conversion of β-Carotene to Canthaxanthin

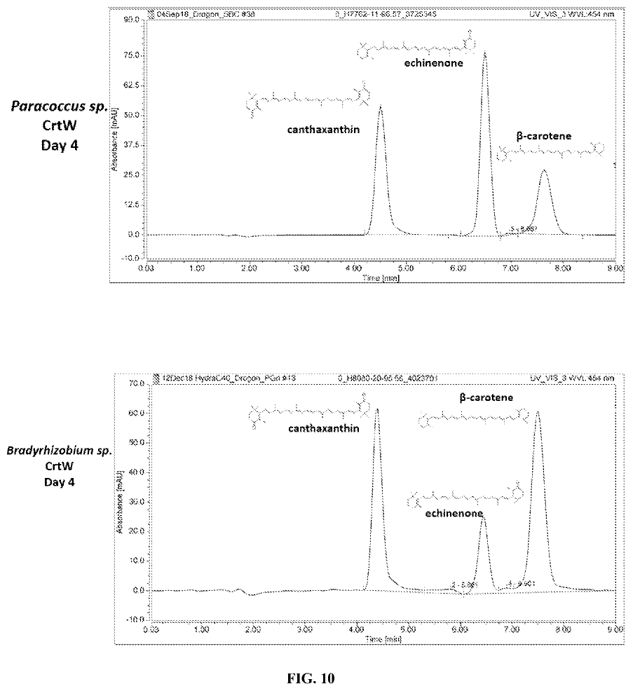

FIG. 9 summarizes production of β-carotene, echinenone, and canthaxanthin in the different strains. Among the 24 different new β-carotene ketolase (CrtW) variants tested, 11 of them were active in the strain background (AMR-13) and produced a measurable amount of canthaxanthin, echinenone, or both. All variants were tested using the same promoter (0.25×pGAL1) used to express the CrtW in AMR-23. Out of the 11 identified CrtW enzyme variants, a β-carotene ketolase from Bradyrhizobium sp. strain ORS278, in strain AMR-27, gave the highest peak area ratio of canthaxanthin to echinenone in bioreactors (FIG. 10 and Table 6-6).

| | | | | Ratio (Day 4) | |
|---|---|---|---|---|---|
| | | | | (Canthaxanthin | |
| | | | Echinenone | peak height/ | β-carotene |
| CrtW species | Canthaxanthin (Day 4) | | (Day 4) | echinenone | (Day 4) |
| source | g/kg DCW | Peak Height | Peak Height | peak height) | g/kg DCW |
| Paracoccus sp. | 1.51 | 52.75 | 76.04 | 0.69 | 0.47 |
| (AMR-23) | | | | | |
| Bradyrhizobium sp. | 1.31 | 54.65 | 23.17 | 2.36 | 0.87 |
| (AMR-27) | | | | | |
| Paracoccus sp. & | 2.25 | 92.64 | 45.05 | 2.06 | 0.46 |
| Bradyrhizobium sp. | | | | | |
| (AMR-28) | | | | | |

Table 6-6: Fermentation performance of three strains (strains AMR-23, AMR-27, and AMR-28) expressing the Paracoccus sp.CrtW (strain AMR-23), Bradyrhizobium sp. (strain AMR-27) or both the combination of Paracoccus sp.CrtW and Bradyrhizobium sp. (strain AMR-28) at Day 4 in 0.5 L fermentation tanks in 8-day, fed batch fermentation runs. All strains have the same parent strain (AMR-13).

The Bradyrhizobium sp. CrtW in AMR-27 appears to complement, instead of replace, the Paracoccus sp. CrtW in AMR-23. The two strains produce similar amounts of canthaxanthin (Table 6-6). However, while AMR-23 effectively performs the first ketolation to echinenone but then is limited by the second to canthaxanthin, AMR-27 appears to effectively convert echinenone to canthaxanthin but is limited by the first ketolation of β-carotene (FIG. 10 and Table 6-6). The two enzymes appear to work in concert to efficiently convert β-carotene to echinenone and then to canthaxanthin.

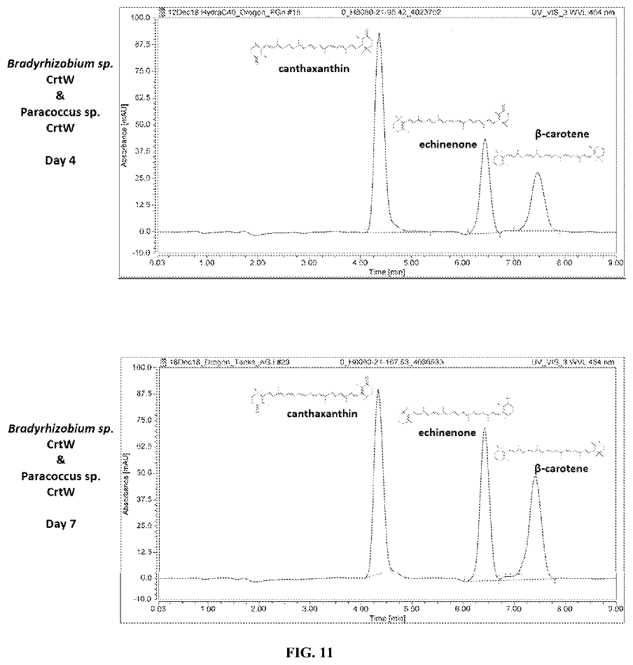

Both the Paracoccus sp. CrtW and the Bradyrhizobium sp. CrtW were co-expressed using two different promoters (0.25× and 0.10× of the strength of pGAL1, respectively) in the β-carotene co-producer strain (AMR-13). The resulting strain, AMR-28, produced an average canthaxanthin titer of 2 g/kg DCW (from Day 4 through Day 8) in an 8-day, fed batch fermentation. The echinenone titer of AMR-28 was significantly reduced compared to parental strain AMR-23, though the concentration of echinenone did increase over the course of the fermentation (FIG. 11). The new strain, AMR-28, achieved one of the highest ratios of canthaxanthin to echinenone with peak titer of 2.25 g/kg DCW canthaxanthin at Day 4 (Table 6-6). Strain AMR-28 had similar farnesene fermentation yield, albeit an 8% reduced volumetric productivity in an 8-day fermentation run compared to the farnesene manufacturing strain (AMR-6; Table 6-2).

The following references are incorporated herein by reference in their entirety:
1. Westfall, P. J. et al. Production of amorphadiene in yeast, and its conversion to dihydroartemisinic acid, precursor to the antimalarial agent artemisinin. Proceedings of the National Academy of Sciences 109, E111-E118 (2012).
2. Meadows, A. L. et al. Rewriting yeast central carbon metabolism for industrial isoprenoid production. Nature 537, 694-697 (2016).
3. Kok, S. de et al. Rapid and Reliable DNA Assembly via Ligase Cycling Reaction. ACS Synthetic Biology 3, 97-106 (2014).
4. Serber, Z., Lowe, R., Ubersax. J. A. & Chandran, S. S. COMPOSITIONS AND METHODS FOR THE ASSEMBLY OF POLYNUCLEOTDES. (2012).
5. Guthrie, C. & Fink, G. R. Methods in enzymology: guide to yeast genetics and molecular biology. (Academic Press, 1991).
6. Chua, P. R., Jiang, H. & Meadows, A. L. U.S. patent Ser. No. 10/808,015—Maltose dependent degrons, maltose-responsive promoters, stabilization constructs, and their use in production of non-catabolic compounds. (2020).
7. Deng, J. et al. A synthetic promoter system for well-controlled protein expression with different carbon sources in Saccharomyces cerevisiae. Microbial Cell Factories 20, 202 (2021).
8. Meadows. A. L. et al. Rewriting yeast central carbon metabolism for industrial isoprenoid production. Nature 537, 694-697 (2016).

One or more features from any embodiments described herein or in the figures may be combined with one or more features of any other embodiment described herein in the figures without departing from the scope of the invention.

All publications, patents and patent applications cited in this specification are herein incorporated by reference as if each individual publication or patent application were specifically and individually indicated to be incorporated by reference. Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

What is claimed:

1. A genetically modified host cell capable of producing canthaxanthin comprising a nucleic acid capable of expressing Bradyrhizobium β-carotene ketolase.

2. The genetically modified host cell of claim 1, wherein the nucleic acid capable of expressing Bradyrhizobium β-carotene ketolase is at least 80, 85, 90, 95, 99, or 100% identical to SEQ ID NO: 1.

3. The genetically modified host cell of claim 1, which comprises Bradyrhizobium β-carotene ketolase.

4. The genetically modified host cell of claim 3, wherein the Bradyrhizobium β-carotene ketolase has an amino acid sequence at least 80, 85, 90, 95, 99, or 100% identical to SEQ ID NO:2.

5. The genetically modified host cell of any of the previous claims, which further comprises a nucleic acid capable of expressing Paracoccus β-carotene ketolase.

6. The genetically modified host cell of claim 5, wherein the nucleic acid capable of expressing Paracoccus β-carotene ketolase is at least 80, 85, 90, 95, 99, or 100% identical to SEQ ID NO: 3.

7. The genetically modified host cell of claim 5, which comprises Paracoccus β-carotene ketolase.

8. The genetically modified host cell of claim 7, wherein the Paracoccus β-carotene ketolase has an amino acid sequence at least 80, 85, 90, 95, 99, or 100% identical to SEQ ID NO:4.

9. The genetically modified host cell of any of the previous claims, which further comprises
(a) a nucleic acid capable of expressing a GGPP synthase;
(b) a nucleic acid capable of expressing a phytoene synthase;
(c) a nucleic acid capable of expressing a phytoene desaturase; and/or
(d) a nucleic acid capable of expressing a lycopene cyclase.

10. The genetically modified host cell of claim 9 wherein one of the nucleic acids encodes a bifunctional enzyme having both the phytoene synthase activity and the lycopene cyclase activity.

11. The genetically modified host cell of claim 9, wherein:
(a) the nucleic acid capable of expressing a GGPP synthase is at least 80, 85, 90, 95, 99, or 100% identical to SEQ ID NO:5;
(b) the nucleic acid capable of expressing a phytoene synthase is at least 80, 85, 90, 95, 99, or 100% identical to SEQ ID NO:7;
(c) the nucleic acid capable of expressing a phytoene desaturase is at least 80, 85, 90, 95, 99, or 100% identical to SEQ ID NO:9; and/or
(d) the nucleic acid capable of expressing a lycopene cyclase is at least 80, 85, 90, 95, 99, or 100% identical to SEQ ID NO:7.

12. The genetically modified host cell of claim 9, wherein:
(a) the GGPP synthase is at least 80, 85, 90, 95, 99, or 100% identical to SEQ ID NO: 6;
(b) the phytoene synthase is at least 80, 85, 90, 95, 99, or 100% identical to SEQ ID NO: 8;
(c) the phytoene desaturase is at least 80, 85, 90, 95, 99, or 100% identical to SEQ ID NO:10; and/or
(d) the lycopene cyclase is at least 80, 85, 90, 95, 99, or 100% identical to SEQ ID NO: 8.

13. The genetically modified host cell of any of the preceding claims, wherein each β-carotene ketolase, GGPP synthase, phytoene synthase, phytoene desaturase, and/or lycopene cyclase is expressed from an inducible promoter.

14. The genetically modified host cell of claim 14, wherein the inducible promoter is a GAL promoter.

15. The genetically modified host cell of claim 14, wherein a GAL80 gene is expressed from a MAL promoter.

16. The genetically modified host cell of any of the previous claims which further comprises a gas4 deletion.

17. The genetically modified host cell of any of the previous claims which further comprises a nucleic acid encoding a polyprenyl synthase for producing a polyprenyl diphosphate.

18. The genetically modified host cell of any of the previous claims which further comprises a nucleic acid encoding a FPP synthase.

19. The genetically modified host cell of any of the previous claims which further comprises one or more nucleic acids encoding one or more enzymes of the mevalonate pathway.

20. The genetically modified host cell of any of the previous claims which further comprises one or more nucleic acids encoding all of the enzymes of the mevalonate pathway.

21. The genetically modified host cell of any of the previous claims which is a microbial cell.

22. The genetically modified host cell of any of the previous claims which is a yeast.

23. The genetically modified host cell of any of the previous claims which is Saccharomyces cerevisiae.

24. A composition comprising the genetically modified host cell of any of the previous claims.

25. A method for producing canthaxanthin comprising the steps:
(a) culturing a population of the host cells of any one of the previous claims in a medium with a carbon source under conditions suitable for making canthaxanthin to yield a culture broth; and
(b) recovering said canthaxanthin from the culture broth.
